(19) United States
(12) Patent Application Publication
(10) Pub. No.: US 2026/0183390 A1
(43) Pub. Date: Jul. 2, 2026

(54) ANTI-IL-2 ANTIBODY COMBINATIONS AND METHODS OF USE THEREOF

(71) Applicants: Aulos Bioscience, Inc., Larkspur, CA (US); Ares Trading SA, Aubonne (CH)

(72) Inventors: Inbar AMIT, Modi'in (IL); Timothy WYANT, Bellingham, MA (US); Yanay OFRAN, Tel Aviv (IL); James Robert Vasselli, San Francisco, CA (US); Aron Marc Knickerbocker, Mill Valley, CA (US)

(73) Assignees: Aulos Bioscience, Inc., Larkspur, CA (US); Ares Trading SA, Aubonne (CH)

(21) Appl. No.: 19/345,459

(22) Filed: Sep. 30, 2025

Related U.S. Application Data
(63) Continuation-in-part of application No. 18/431,983, filed on Feb. 4, 2024.
Application No. 18/431,983 is a continuation-in-part of application No. 18/404,093, filed on Jan. 4, 2024.
Application No. 18/404,093 is a continuation-in-part of application No. PCT/US2023/079221, filed on Nov. 9, 2023.
(60) Provisional application No. 63/383,086, filed on Nov. 10, 2022.
(60) Provisional application No. 63/503,481, filed on May 21, 2023.
(60) Provisional application No. 63/503,977, filed on May 24, 2023.
(60) Provisional application No. 63/589,659, filed on Oct. 12, 2023.

(51) Int. Cl. A61K 39/395; A61K 38/20; A61K 39/00; A61P 35/04

(52) U.S. Cl. CPC A61K 39/3955; A61K 38/2013; A61P 35/04; A61K 2039/507; A61K 2039/545

(57) ABSTRACT

Described herein are combination therapies comprising engineered anti-IL-2 antibodies in combination with an immune checkpoint inhibitor such as avelumab and an initial low dose of IL-2 and related therapeutic methods for a subjects with cancer, including a solid tumor and specifically non-small cell lung cancer (NSCLC).

### CROSS-REFERENCE TO RELATED APPLICATIONS

This application is a Continuation-in-Part Application of U.S. application Ser. No. 18/431,983 filed Feb. 4, 2024, which is a Continuation-in-Part application of U.S. application Ser. No. 18/404,093 filed Jan. 4, 2024, which is a Continuation-in-Part application of PCT International Application No. PCT/US23/79221, International Filing Date Nov. 9, 2023, claiming the benefit of priority of U.S. Provisional Application No. 63/589,659 filed Oct. 12, 2023, U.S. Provisional Application No. 63/503,977 filed May 24, 2023, U.S. Provisional Application No. 63/503,481 filed May 21, 2023, and U.S. Provisional Application No. 63/383,086 filed Nov. 10, 2022, which are all hereby incorporated by reference in their entirety.

### SEQUENCE LISTING STATEMENT

The instant application contains a Sequence Listing which has been submitted electronically in XML format and is hereby incorporated by reference in its entirety. The XML formatted sequence listing, created on Mar. 20, 2026, is named P-621284-US3.XML and is 98.2 kilobytes in size.

### FIELD OF THE INVENTION

The disclosure relates in general to the field of cancer treatment. In some embodiments, the present disclosure describes a combination therapy comprising engineered anti-IL-2 antibodies, low dose IL-2, and an immune checkpoint inhibitor such as avelumab and the use of the combination for treating cancers, including solid tumors and specifically non-small cell lung cancer (NSCLC).

### BACKGROUND

Interleukin-2 (IL-2), a potent T-cell-stimulating cytokine, was the first U.S. Food and Drug Administration (FDA)-approved immunotherapeutic effective in treating cancers such as metastatic melanoma and renal cell carcinoma (RCC). However, the need for high doses and frequent administration of IL-2 to induce therapeutic effects has led to severe side and off-target effects.

IL-2 signaling has two opposite effects. IL-2 can enhance immune response by activation of effector cells and induce their proliferation. Alternatively, IL-2 can tune down immune response by activation and proliferation of CD4+ regulatory T (Treg) cells. To facilitate these functions, IL-2 mediates its effect by binding to two forms of IL-2 receptor: i) trimeric receptors made up of IL-2Rα (CD25), IL-2Rβ (CD122), and a common IL-2Rγ (γc, CD132) chains, or ii) a dimeric receptor that consists of only the IL-2Rβ and IL-2Rγ subunits. Both the dimeric and trimeric receptors are able to transmit IL-2 binding signaling via the STAT5 pathway. However, IL-2 binds the αβγ receptor trimer at 100-fold tighter than the βγ receptor dimer. It has been demonstrated that the binding affinity of hIL-2 to the αβγ trimer is approximately 10 pM, whereas the hIL-2 affinity to the βγ dimer is 1 nM.

AU-007 is a human IgG1 monoclonal antibody (mAb) that binds to IL-2 with pM affinity on its CD25 binding epitope and completely inhibits its binding to CD25, without hindering its binding to CD132/CD122.

AU-007-bound IL-2 cannot bind trimeric (CD25, CD122, CD132) IL-2 receptors (IL-2R) on regulatory T cells (Tregs), vascular endothelium, or eosinophils, but IL-2's binding to dimeric (CD122, CD132) IL-2R on effector T (T eff) and NK cells is unhindered. AU-007 thus redirects IL-2 towards T eff and NK cell activation, while diminishing Treg activation and vascular leak, and redirects IL-2 generated from T eff cell expansion, converting a Treg-mediated autoinhibitory loop into an immune stimulating loop, driving tumor killing.

AU-007 bound IL-2 prolongs the T_{1/2} of IL-2, allowing endogenous IL-2 or low dose aldesleukin to initiate an anti-tumor response. AU-007 monotherapy at doses up to 12 mg/kg Q2W is safe and well tolerated, with initial signs of immune modulation consistent with AU-007's mechanism of action. More work is needed to ascertain how to optimize the benefits of treatment with AU-007 in combination with IL-2 in patients.

Blocking the programmed cell death-1 (PD-1)/programmed death ligand-1 (PD-L1) pathway through monoclonal antibodies (mAbs) is a proven therapeutic strategy against multiple solid tumors, including non-small cell lung cancer (NSCLC). Avelumab is a human IgG1 anti-PD-L1 mAb that is approved for use against several solid tumor histologies. In addition to blocking the PD-1/PD-L1 pathway, avelumab contains an active fragment crystallizable (Fc) moiety that triggers antibody-dependent cell-mediated cytotoxicity (ADCC), including NK cell mediated cytotoxicity, bringing both the innate and adaptive immune systems to bear against tumors.

Avelumab has been investigated for non-small cell lung cancer (NSCLC), showing antitumor activity in Phase 1 and 3 trials (JAVELIN Lung 100, JAVELIN Lung 200). While avelumab was not superior to chemotherapy in improving overall survival (OS) in the first-line (JAVELIN Lung 100) or second-line (JAVELIN Lung 200) settings, it demonstrated potential in certain patient groups, particularly those with high PD-L1 expression. While avelumab has shown promising antitumor activity and an acceptable safety profile in NSCLC, its efficacy as a standalone first-line or second-line treatment has not consistently met primary survival endpoints compared to standard chemotherapy. Therefore, more work is needed to ascertain how to optimize the benefits of treatment with avelumab as well.

### SUMMARY

In one aspect, disclosed herein is a combination therapy comprising an anti-IL-2 antibody or a pharmaceutical composition thereof, a loading dose of a low dose of IL-2 or a pharmaceutical composition thereof, and avelumab or a pharmaceutical composition thereof, wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:62, said HCDR2 comprises the amino acid sequence of SEQ ID NO: 63, said HCDR3 comprises the amino acid sequence of SEQ ID NO:64, said LCDR1 comprises the amino acid sequence of SEQ ID NO:65, said LCDR2 comprises the amino acid sequence of DAS, and said LCDR3 comprises the amino acid sequence of SEQ ID NO: 67; wherein said anti-IL-2 antibody is formulated for administration at a dose of 9 mg/kg of a subject's body weight, the loading low dose of IL-2 is formulated for subcutaneous injection and administration at a dose of 135,000 IU/kg of a subject's body weight, and said avelumab is formulated for administration at a dose of 800 mg wherein said pharmaceutical composition further comprises a pharmaceutically acceptable carrier. In a related aspect, this combination therapy is for treating unresectable locally advanced or metastatic non-small-cell lung cancer (NSCLC) that is PD-L1 positive. In a further related aspect, this combination therapy is for 2^{nd}- or 3^{rd}-line treatment.

In another related aspect of the combination therapy, the amino acid sequence of the VH comprises the amino acid sequence of SEQ ID NO:26 and the amino acid sequence of VL comprises the amino acid sequence of SEQ ID NO:27. In a further related aspect, the amino acid sequence of the full length heavy chain is set forth in SEQ ID NO:72 and the amino acid sequence of the full length light chain is set forth in SEQ ID NO:73. In another related aspect of the combination therapy, the antibody comprises an IgG, IgA, IgM, IgE, IgD, a Fv, a scFv, a Fab, a F(ab′) 2, a minibody, a diabody, or a triabody. In a further related aspect, the antibody comprises a heavy chain comprising a mutation that reduces binding to fragment crystallizable gamma receptors (FcγRs), wherein reduced binding is compared with said antibody lacking the mutation in the heavy chain that affects FcγRs binding. In yet another further related aspect, the mutation comprises L234A, L235A (LALA) mutations.

In another aspect, disclosed herein is a method of treating an unresectable locally advanced or metastatic non-small-cell lung cancer (NSCLC) that is PD-L1 positive in a subject, said method comprising administering to said subject a combination therapy comprising an anti-IL-2 antibody at a dose of 9 mg/kg of said subject's body weight or a pharmaceutical composition thereof, a loading dose of 135,000 IU/kg of said subject's body weight of IL-2 or a pharmaceutical composition thereof, and avelumab at a dose of 800 mg or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:62, said HCDR2 comprises the amino acid sequence of SEQ ID NO:63, said HCDR3 comprises the amino acid sequence of SEQ ID NO:64, said LCDR1 comprises the amino acid sequence of SEQ ID NO:65, said LCDR2 comprises the amino acid sequence of DAS, and said LCDR3 comprises the amino acid sequence of SEQ ID NO:67, wherein said pharmaceutical composition(s) further comprises a pharmaceutically acceptable carrier, and wherein said treating comprises second-line or third-line treatment, thereby treating said unresectable locally advanced or metastatic NSCLC that is PD-L1 positive NSCLC in said subject.

In a related aspect of a method of treating an unresectable locally advanced or metastatic NSCLC, the administration of said loading dose of IL-2 is prior to, concurrent with, or following the administration of said anti-IL-2 antibody, said avelumab, or both.

In another related aspect of method of treating an unresectable locally advanced or metastatic NSCLC, the method further comprises the step of administering one or more additional doses of said anti-IL-2 antibody or a pharmaceutical composition thereof, wherein said pharmaceutical composition further comprises a pharmaceutically acceptable carrier.

In another related aspect of method of treating an unresectable locally advanced or metastatic NSCLC, the method comprises administering additional doses of said anti-IL-2 antibody or a pharmaceutical composition thereof are administered to said subject once every two weeks. In yet another related aspect of method of treating an unresectable locally advanced or metastatic NSCLC, the method comprises the step of administering one or more additional doses of said avelumab or a pharmaceutical composition thereof, wherein said pharmaceutical composition further comprises a pharmaceutically acceptable carrier. In a further related aspect, the one or more additional doses of said avelumab or a pharmaceutical composition thereof are administered to said subject once every two weeks.

In another related aspect of method of treating an unresectable locally advanced or metastatic NSCLC, the method further comprises the step of administering one or more booster doses of IL-2 or a pharmaceutical composition thereof, wherein said pharmaceutical composition further comprises a pharmaceutically acceptable carrier. In a further related aspect, the administration of said one or more booster doses of IL-2 is prior to, concurrent with, or following the administration of said one or more additional doses of said anti-IL-2 antibody, one or more additional doses of said avelumab, or both. In another further related aspect, at least one of the one or more IL-2 booster doses is administered at a dose of 135,000 IU/kg of said subject's body weight. In yet another further related aspect, the at least one booster dose is administered to said subject if tumor volume is stable, if one or more previously shrinking tumors becomes stable, if there is an increase in one or more tumor markers, if one or more new tumors are detected, if tumor growth is detected in one or more tumors that had previously been stable or had decreased in size, or any combination thereof.

In another related aspect of method of treating an unresectable locally advanced or metastatic NSCLC, the method comprises (i) reducing the size of the tumor, (ii) inhibiting or reducing growth of the tumor, (iii) inhibiting or reducing metastases of said tumor, (iv) inhibiting the production of new lesions, (v) decreasing or shrinking target lesions, (vi) eliminating target lesions, or (vii) any combination thereof. In a further related aspect, said NSCLC progressed after prior checkpoint inhibitor therapy in said subject. In yet another further related aspect, said NSCLC comprises squamous NSCLC. In other further related aspects, said NSCLC comprises non-squamous NSCLC.

### BRIEF DESCRIPTION OF THE DRAWINGS

The present disclosure of engineered anti-IL-2 antibody and combination therapies comprising the anti-IL-2 antibody, both as to their generation and method of use, together with objects, features, and advantages thereof, may best be understood by reference to the following detailed description when read with the accompanying drawings in which:

FIG. 1 graphically presents the administration and dosage schemes of AU-007 Q2w monotherapy (left—1A), AU-007+a single IL-2 Loading Dose (center—1B), and combination therapy of AU-007 Q2w+IL-2 Q2w (right—1C). When administered, recombinant human IL-2 (aldesleukin) was administered subcutaneously, at much lower doses and much less frequently than the approved regimen of intravenously administered aldesleukin. The terms “3+3” and “1+2” refer to the size of the cohort at a given dose level. For 3+3, the first 3 enrolled patients were administered AU-007 at the dose level that is noted. If no dose-limiting toxicities (DLTs) were seen, then administration was escalated to the next highest dose level. However, if any of the first 3 patients had a dose-limiting toxicity that was drug-related, then an additional three more patients would have received treatment at that same dose level to see if they have DLTs before escalating. 1+2 follows the same principle—start with one patient, if DLTs were observed, add two more, or if no DLTs were observed in the first single patient, then dosages can be escalated.

FIG. 2 provides a timeline of the treatment with AU-007 in combination with single-dose aldesleukin and optional aldesleukin boost administration and tumor assessment. AU-007 is administered every 2 weeks (Day 1, 15, 29, 43—see black arrow); Aldesleukin is administered as a loading dose once in Cycle 1 (Day 1—see ellipse). Tumor evaluation (black diamond) using Response Evaluation Criteria In Solid Tumors (RECIST) evaluation of CT scan, MRI, PET scan, or ultrasound to measure existing tumor size and assess for any new tumors is carried out at the end of each cycle. Biopsy (4-point star) is conducted to evaluate immune-modulating effect of drug within tumor by evaluating immune cell infiltration. The Dose-Limiting Toxicity (DLT) period lasts 4 weeks and informs dose escalation decisions. The aldesleukin boost administration is administered in patients who are tolerating treatment and are clinically stable at the discretion of the Investigator and Sponsor based on the following: (1) with each cycle (Q8W, Day 1 of cycle) until objective tumor shrinkage is observed on radiologic imaging or physical exam or (2) with objective signs of worsening tumor growth kinetics: e.g., previously shrinking tumors becoming stable, increase in tumor markers, tumor growth, or appearance of new tumor growth in a tumor that had previously been stable or had decreased in size, or any combination thereof.

FIGS. 3A-3B show differences in peripheral cell population (Treg, NK, CD8+ cell populations, and the CD8/Treg ratio) measured on Day 15 (FIG. 3A) or Day 44 (FIG. 3B) across all cohorts dosed with any AU-007 (e.g., 4.5 mg/kg AU-007) and escalating doses of subcutaneous aldesleukin (15,000 IU/kg, 45,000 IU/kg, 135,000 IU/kg, or 270,000 IU/kg).

FIG. 4 shows a comparison of progression-free survival (PFS) between patients with less than 43% reduction in Tregs vs. greater than 43% Tregs reduction, where 43% reduction is the median Treg reduction.

FIG. 5 shows the dose response effect of subcutaneous aldesleukin administration and the concentration of circulating IFN-γ.

FIG. 6 shows dose response effect of IV AU-007 administration and the concentration of circulating IFN-γ. On Day 1 (0-24 hours), IFN-γ from blood at 5 different timepoints (pre-dose, 2 hours, 6 hours, 24 hours and 48 hours post dose) was measured; on Day 15 (360 hours), IFN-γ from blood at 3 different timepoints (pre-dose, end of infusion, and 6 hours post-infusion) was measured; on Day 29 (700 hours), IFN-γ from blood at 2 different timepoints (pre-dose and end of infusion) was measured.

FIG. 7 shows the relative risk of progression as a function of IL-2 dose (left hand side) and AU-007 dose (right hand side) for renal cell carcinoma (RCC) (middle row), melanoma (bottom row), and other cancers (top row).

FIG. 8 AU-007 PK and IL-2 Coverage Calculated on a Mole to Mole Ratio.

FIGS. 9A-9B show the best response in melanoma patients in Phase 1 and Phase 2 receiving AU-007 Q2W+Single SC Aldesleukin (Arm B regimen; FIG. 9A) or AU-007 Q2W+SC Aldesleukin Q2W (Arm C regimen; FIG. 9B). In the Arm B regimen, AU-007 was administered IV at 4.5 mg/kg and in the Arm C regimen, AU-007 was administered IV at 4.5 or 9 mg/kg. For both arms, Aldesleukin was administered SC at a dose of 15 IU/kg, 45 IU/kg, 135 IU/kg, or 270K IU/kg.

FIGS. 10A-10B show the percentage change over time in sum of diameters in target lesions vs. baseline in Melanoma Patients in Phase 1 and Phase 2 patients receiving AU-007 Q2W+Single SC Aldesleukin loading dose (Arm B Regimen; FIG. 10A) or AU-007 Q2W+SC Aldesleukin Q2W (Arm C regimen; FIG. 10B). In the Arm B regimen, AU-007 was administered IV at 4.5 mg/kg and in the Arm C regimen, AU-007 was administered IV at 4.5 or 9 mg/kg. For both arms, Aldesleukin was administered SC at a dose of 15 IU/kg, 45 IU/kg, 135 IU/kg, or 270K IU/kg.

FIGS. 11A-11B show the best response in RCC patients in Phase 1 and Phase 2 receiving the AU-007 Q2W+Single SC Aldesleukin (Arm B regimen; FIG. 11A) or AU-007 Q2W+SC Aldesleukin Q2W (Arm C regimen; FIG. 11B).

FIG. 12 shows the percentage change over time in sum of diameters in target lesions vs. baseline in RCC Patients in Phase 1 and Phase 2 receiving AU-007 Q2W+SC aldesleukin loading dose (Arm B Dosing Regimen).

FIG. 13 shows the probability of Progression-Free Survival (PFS) for all Phase 1 and 2 patients with all tumor types, comparing Arm B Regimen vs. Arm C Regimen.

FIGS. 14A and 14B show Treg fold change vs. baseline by Week 8 (FIG. 14A) and Week 16 (FIG. 14B) progression status. The boxplots summarize maximum Treg decrease for each arm and progression status group showing median (line in each box) with lower and upper box boundaries corresponding to the 25th and 75th percentile.

FIGS. 15A-15C show the percent change in Tregs (FIG. 15A) and CD8 Cells (FIG. 15B), and the CD8/Treg ratio (FIG. 15C) normalized to baseline in Arm 2B vs. Arm 2C regimens.

FIG. 16 provides a timeline of the treatment with AU-007 in combination with single-dose aldesleukin and avelumab and optional aldesleukin boost administration and tumor assessment. Cycles are 8 weeks (56 days). AU-007 and avelumab are administered 4 times in a cycle (Day 1, 15, 29, 43—see black arrow and black rectangle, respectively); Aldesleukin is administered as a loading dose once in Cycle 1 (Day 1—see ellipse). Tumor evaluation (black diamond) using RECIST evaluation of CT scan, MRI, PET scan, or ultrasound to measure existing tumor size and assess for any new tumors is carried out at the end of each cycle. Biopsy (4-point star) is conducted to evaluate immune-modulating effect of drug within tumor by evaluating immune cell infiltration. The Dose-Limiting Toxicity (DLT) period lasts 4 weeks and informs dose escalation decisions. The aldesleukin boost administration (not shown) is administered in patients who are tolerating treatment and are clinically stable at the discretion of the Investigator and Sponsor based on the following: (1) with each cycle (Q8W, Day 1 of cycle) until objective tumor shrinkage is observed on radiologic imaging or physical exam or (2) with objective signs of worsening tumor growth kinetics: e.g., previously shrinking tumors becoming stable, increase in tumor markers, tumor growth, or appearance of new tumor growth in a tumor that had previously been stable or had decreased in size, or any combination thereof. If a patient continues to subsequent cycles, they begin with AU-007 and avelumab on Cycle 2 Day 1, then AU-007 administered with avelumab on Days 15, 29, and 43 of Cycle 2; and so on.

FIGS. 17A-17C show tumor diameter data over time in 3 patients (Patient AU08-0066, FIG. 17A, Patient US01-0098, FIG. 17B, and Patient AU03-0084, FIG. 17C) after receiving 9 mg/kg IV AU-007 Q2W and 135,000 IU/kg SC aldesleukin (IL-2) loading dose. Patient AU08-0066 (FIG. 17A) received a booster dose of aldesleukin on Cycle 4 Day 1 (C4D1; 24 weeks since treatment initiation). Patient US01-0098 (FIG. 17B) received two booster doses of aldesleukin on Cycle 2 Day 1 (C2D1; 8 weeks since treatment initiation); and on Cycle 3 Day 1 (C3D1; 16 weeks since treatment initiation). Patient AU03-0084 (FIG. 17C) received one booster dose of aldesleukin on Cycle 2 Day 1 (C2D1; 8 weeks since treatment initiation).

### DETAILED DESCRIPTION

In the following detailed description, numerous specific details are set forth in order to provide a thorough understanding of the antibodies disclosed herein. However, it will be understood by those skilled in the art that preparation and uses of antibodies disclosed herein may in certain cases be practiced without these specific details. In other instances, well-known methods, procedures, and components have not been described in detail so as not to obscure the disclosure presented herein.

Throughout this application, various references or publications are cited. Disclosures of these references or publications in their entireties are hereby incorporated by reference into this application in order to more fully describe the state of the art to which this invention pertains.

As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen.

By “specifically binding” is meant that the binding is selective for the antigen of interest and can be discriminated from unwanted or nonspecific interactions. For example, an antibody is said to specifically bind an IL-2 epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹²M. In some embodiments, the equilibrium dissociation constant may be in the range of ≤10⁻⁵ M to 10⁻¹²M.

As used herein, the term “antibody” encompasses an antibody fragment or fragments that retain binding specificity including, but not limited to, IgG, heavy chain variable region (VH), light chain variable region (VL), Fab fragments, F(ab′) 2 fragments, scFv fragments, Fv fragments, a nanobody, minibodies, diabodies, triabodies, tetrabodies, and single domain antibodies (see, e.g., Hudson and Souriau, Nature Med. 9:129-134 (2003)). Also encompassed are humanized, primatized, and chimeric antibodies as these terms are generally understood in the art.

A skilled artisan would appreciate that in certain embodiments, the term “anti-IL-2 antibody” as used herein is interchangeable with the term “anti-human-IL-2 antibody”, having all the same qualities and meanings. Similarly, as used throughout, in certain embodiments, the term “IL-2” is interchangeable with the term “human IL-2”, having all the same qualities and meanings.

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs, and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each of a heavy or light chain polypeptide has three CDRs denoted as “CDR1”, “CDR2”, and “CDR3”. Crystallographic analysis of a number of antigen-antibody complexes has demonstrated that the amino acid residues of CDRs form extensive contact with a bound antigen, wherein the most extensive antigen contact is with the heavy chain CDR3. Thus, the CDR regions are primarily responsible for the specificity of an antigen-binding site. In some embodiments, an antigen-binding site includes six CDRs, comprising the CDRs from each of a heavy and a light chain variable region.

As used herein, the term “framework region” or “FR” refers to the four flanking amino acid sequences which frame the CDRs of a heavy or light chain variable region. Some FR residues may contact bound antigen; however, FR residues are primarily responsible for folding the variable region into the antigen-binding site. In some embodiments, the FR residues responsible for folding the variable regions comprise residues directly adjacent to the CDRs. Within FRs, certain amino residues and certain structural features are very highly conserved. In this regard, all variable region sequences contain an internal disulfide loop of around 90 amino acid residues. When a variable region folds into an antigen binding site, the CDRs are displayed as projecting loop motifs that form an antigen-binding surface. It is generally recognized that there are conserved structural regions of FR that influence the folded shape of the CDR loops into certain “canonical” structures regardless of the precise CDR amino acid sequence. Furthermore, certain FR residues are known to participate in non-covalent interdomain contacts which stabilize the interaction of the antibody heavy and light chains.

Wu and Kabat (Tai Te Wu, Elvin A. Kabat. An analysis of the sequences of the variable regions of bence jones proteins and myeloma light chains and their implications for antibody complementarity. Journal of Experimental Medicine, 132, 2, 8 (1970); Kabat EA, Wu TT, Bilofsky H, Reid-Miller M, Perry H. Sequence of proteins of immunological interest. Bethesda: National Institute of Health; 1983. 323 (1983)) pioneered the alignment of antibody peptide sequences, and their contributions in this regard were several-fold: Firstly, through study of sequence similarities between variable domains, they identified correspondent residues that to a greater or lesser extent were homologous across all antibodies in all vertebrate species, inasmuch as they adopted similar three-dimensional structure, played similar functional roles, interacted similarly with neighboring residues, and existed in similar chemical environments. Secondly, they devised a peptide sequence numbering system in which homologous immunoglobulin residues were assigned the same position number. One skilled in the art can unambiguously assign to any variable domain sequence what is now commonly called Kabat numbering without reliance on any experimental data beyond the sequence itself. Thirdly, Kabat and Wu calculated variability for each Kabat-numbered sequence position, by which is meant the finding of few or many possible amino acids when variable domain sequences are aligned. They identified three contiguous regions of high variability embedded within four less variable contiguous regions. Kabat and Wu formally demarcated residues constituting these variable tracts, and designated these “complementarity determining regions” (CDRs), referring to chemical complementarity between antibody and antigen. A role in three-dimensional folding of the variable domain, but not in antigen recognition, was ascribed to the remaining less-variable regions, which are now termed “framework regions”. Fourth, Kabat and Wu established a public database of antibody peptide and nucleic acid sequences, which continues to be maintained and is well known to those skilled in the art.

Chothia and coworkers (Cyrus Chothia, Arthur M. Lesk. Canonical structures for the hypervariable regions of immunoglobulins. Journal of Molecular Biology, 196, 4, 8 (1987)) found that certain sub portions within Kabat CDRs adopt nearly identical peptide backbone conformations, despite having great diversity at the level of amino acid sequence. These sub portions were designated as L1, L2 and L3 or H1, H2 and H3, where the “L” and the “H” designates the light chain and the heavy chains regions, respectively. These regions may be referred to as Chothia CDRs, which have boundaries that overlap with Kabat CDRs.

More recent studies have shown that virtually all antibody binding residues fall within regions of structural consensus (Kunik, V. et al., PloS Computational Biology 8 (2): el002388 (February 2012)). In some embodiments, these regions are referred to as antibody binding regions. It was shown that these regions can be identified from the antibody sequence as well. “Paratome”, an implementation of a structural approach for the identification of structural consensus in antibodies, was used for this purpose. (Ofran, Y. et al., J. Immunol. 757:6230-6235 (2008)). While residues identified by Paratome cover virtually all the antibody binding sites, the CDRs (as identified by the commonly used CDR identification tools) miss significant portions of them. Antibody binding residues which were identified by Paratome but were not identified by any of the common CDR identification methods are referred to as Paratome-unique residues. Similarly, antibody binding residues that are identified by any of the common CDR identification methods but are not identified by Paratome are referred to as CDR-unique residues. Paratome-unique residues make crucial energetic contributions to antibody-antigen interactions, while CDRs-unique residues make a rather minor contribution. These results allow for better identification of antigen binding sites.

IMGT® is the international ImMunoGene Tics information System®, (See, Nucleic Acids Res. 2015 January; 43 (Database issue): D413-22. doi: 10.1093/nar/gku1056. Epub 2014 Nov. 5 Free article. PMID: 25378316 LIGM: 441 and Dev Comp Immunol. 2003 January; 27 (1): 55-77). IMGT is a unique numbering system for immunoglobulin and T cell receptor variable domains and Ig superfamily V-like domains (Lefranc et al., Dev Comp Immunol. 27:55-77 (2003)). IMGT® presents a uniform numbering system for these IG and TcR variable domain sequences, based on aligning 5 or more IG and TcR variable region sequences, taking into account and combining the Kabat definition of FRs and CDRs, structural data, and Chothia's characterization of the hypervariable loops. IMGT is considered well known in the art as a universal numbering scheme for antibodies.

In some embodiments, identification of potential variant amino acid positions in the VH and VL domains uses the IMGT system of analysis. In some embodiments, identification of potential variant amino acid positions in the VH and VL domains uses the Paratome system of analysis. In some embodiments, identification of potential variant amino acid positions in the VH and VL domains uses the Kabat system of analysis. In some embodiments, identification of potential variant amino acid positions in the VH and VL domains uses the Clothia system of analysis.

In describing variant amino acid positions present in the VH and VL domains, in some embodiments the IMGT numbering is used. In describing variant amino acid positions present in the VH and VL domains, in some embodiments the Paratome numbering is used. In describing variant amino acid positions present in the VH and VL domains, in some embodiments the Kabat numbering is used. In describing variant amino acid positions present in the VH and VL domains, in some embodiments the Clothia numbering is used.

Antigen binding sequences are conventionally located within the heavy chain and light chain variable regions of an antibody. These heavy and light chain variable regions may, in certain instances, be manipulated to create new binding sites, for example to create antibodies or fragments thereof, that bind to a different antigen or to a different epitope of the same antigen. In some embodiments, as described herein, manipulating the sequences of a heavy chain variable region or the sequences of a light chain variable region, or both, would create a new binding site for a second antigen.

An antibody may exist in various forms or having various domains including, without limitation, a complementarity determining region (CDR), a variable region (Fv), a VH domain, a VL domain, a single chain variable region (scFv), and a Fab fragment.

A person of ordinary skill in the art would appreciate that a scFv is a fusion polypeptide comprising the variable heavy chain (VH) and variable light chain (VL) regions of an immunoglobulin, connected by a short linker peptide, the linker may have, for example, 10 to about 25 amino acids.

A skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)₂ comprises a fragment of a heavy chain comprising a VH domain and a light chain comprising a VL domain.

In some embodiments, an antibody encompasses whole antibody molecules, including monoclonal and polyclonal antibodies. In some embodiments, an antibody encompasses an antibody fragment or fragments that retain binding specificity including, but not limited to, variable heavy chain (VH) fragments, variable light chain (VL) fragments, Fab fragments, F(ab′) 2 fragments, scFv fragments, Fv fragments, minibodies, diabodies, triabodies, and tetrabodies.

### Methods of Use

In some embodiments, the present disclosure provides methods of treating a cancer in a subject comprising administering to the subject a combination therapy comprising an anti-IL-2 antibody or a pharmaceutical composition thereof, a loading dose of IL-2 or a pharmaceutical composition thereof, and avelumab or a pharmaceutical composition thereof. In some embodiments, the present disclosure provides methods of treating a solid tumor.

In some embodiments, the present disclosure provides methods of treating an unresectable locally advanced or metastatic solid cancer in a subject comprising administering to the subject a combination therapy comprising an anti-IL-2 antibody or a pharmaceutical composition thereof, a loading dose of IL-2 or a pharmaceutical composition thereof, and avelumab or a pharmaceutical composition thereof.

In some embodiments, the present disclosure provides methods of treating an unresectable locally advanced or metastatic non-small cell lung cancer (NSCLC) in a subject comprising administering to the subject a combination therapy comprising an anti-IL-2 antibody or a pharmaceutical composition thereof, a loading dose of IL-2 or a pharmaceutical composition thereof, and avelumab or a pharmaceutical composition thereof. In some embodiments, the NSCLC is PD-L1 positive. In some embodiments, the NSCLC is PD-1 positive. In some embodiments, the NSCLC progressed after prior checkpoint inhibitor therapy in the subject. In some embodiments, the NSCLC comprises squamous NSCLC. In some embodiments, the NSCLC comprises non-squamous NSCLC.

In some embodiments, the present disclosure provides methods of treating an unresectable locally advanced or metastatic non-small cell lung cancer (NSCLC) that is PD-L1 positive, in a subject comprising administering to the subject a combination therapy comprising an anti-IL-2 antibody or a pharmaceutical composition thereof, a loading dose of IL-2 or a pharmaceutical composition thereof, and avelumab or a pharmaceutical composition thereof. In some embodiments, the present disclosure provides methods of treating an unresectable locally advanced or metastatic non-small cell lung cancer (NSCLC) that is PD-1 positive, in a subject comprising administering to the subject a combination therapy comprising an anti-IL-2 antibody or a pharmaceutical composition thereof, a loading dose of IL-2 or a pharmaceutical composition thereof, and avelumab or a pharmaceutical composition thereof. In some embodiments, the NSCLC progressed after prior checkpoint inhibitor therapy in the subject. In some embodiments, the NSCLC comprises squamous NSCLC. In some embodiments, the NSCLC comprises non-squamous NSCLC.

In some embodiments, the present disclosure provides methods of treating melanoma, renal cell carcinoma, non-small cell lung cancer or other cancer conditions.

In some embodiments, the present disclosure provides methods of treating a melanoma, a metastatic melanoma, a primary melanoma and metastatic melanoma, a renal cell carcinoma (RCC), a non-small cell lung cancer (NSCLC), a nasopharyngeal carcinoma, a urothelial cancer, an adrenal cortical carcinoma, a clear cell renal cell carcinoma (ccRCC), a triple-negative breast cancer, a head and neck cancer, a head and neck squamous cell carcinoma (HNSCC), a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a bladder cancer, a uterine cancer, a gallbladder cancer, or a Merkel cell carcinoma. In other embodiments, the present disclosure provides methods of treating or preventing a condition caused by IL-2 binding to endothelial CD25 expressing cells, e.g., pulmonary edema, or IL-2-induced vascular leakage.

In other embodiments, the present disclosure provides methods of treating a solid tumor. In some embodiments, a solid tumor comprises a head and neck cancer, a head and neck squamous cell carcinoma (HNSCC), a pancreatic cancer, a lung cancer, a thyroid cancer, a non-small cell lung cancer (NSCLC), a nasopharyngeal carcinoma, a melanoma, an acral melanoma, a uveal melanoma, a colorectal cancer (CRC), a bladder cancer, cholangiocarcinoma (bile duct cancer), a uterine cancer, a cervical cancer, a gallbladder cancer, a cutaneious squamous carcinoma, or a renal cell carcinoma (RCC). In some embodiments, a solid tumor comprises a head and neck cancer, a pancreatic cancer, or a non-small cell lung cancer. In some embodiments, a solid tumor comprises a non-small cell lung cancer (NSCLC), a melanoma, a metastatic melanoma, a primary melanoma and a metastatic melanoma, or a renal cell carcinoma (RCC). In some embodiments, a solid tumor comprises a melanoma, a metastatic melanoma, a primary melanoma and metastatic melanoma, a renal cell carcinoma (RCC), a non-small cell lung cancer (NSCLC), a nasopharyngeal carcinoma, a urothelial cancer, an adrenal cortical carcinoma, a clear cell renal cell carcinoma (ccRCC), a triple-negative breast cancer, a head and neck cancer, a head and neck squamous cell carcinoma (HNSCC), a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a bladder cancer, a uterine cancer, a gallbladder cancer, or a Merkel cell carcinoma.

In some embodiments, a solid tumor comprises a head and neck cancer. In some embodiments, a solid tumor comprises a pancreatic cancer. In some embodiments, a solid tumor comprises a lung cancer. In some embodiments, a solid tumor comprises a thyroid cancer. In some embodiments, a solid tumor comprises a nasopharyngeal carcinoma. In some embodiments, a solid tumor comprises a melanoma. In some embodiments, a melanoma comprises an acral melanoma or a uveal melanoma. In some embodiments, a solid tumor comprises a colorectal cancer (CRC). In some embodiments, a solid tumor comprises a bladder cancer. In some embodiments, a solid tumor comprises cholangiocarcinoma (bile duct cancer). In some embodiments, a solid tumor comprises a uterine cancer. In some embodiments, a solid tumor comprises a cervical cancer. In some embodiments, a solid tumor comprises a gallbladder cancer. In some embodiments, a solid tumor comprises a renal cell carcinoma (RCC). In some embodiments, a head and neck cancer is a head and neck squamous carcinoma (HNSCC1). In some embodiments, a CRC has high MSI. In some embodiments, a melanoma has a wild-type BRAF gene. In some embodiments, a melanoma has a mutant BRAF gene. In some embodiments, a pancreatic cancer is an adenocarcinoma. In some embodiments, a solid tumor comprises a non-small cell lung cancer (NSCLC). In some embodiments, a NSCLC is a squamous cancer. In some embodiments, a NSCLC is a non-squamous cancer. In some embodiments, a NSCLC has a mutated epidermal growth factor receptor (EGFRm). In some embodiments, a solid tumor comprises a cutaneous squamous carcinoma.

In some embodiments, the present disclosure provides methods of treating an NSCLC in a subject. In some embodiments, the present disclosure provides a method of treating an unresectable locally advanced or metastatic NSCLC. In some embodiments, a method of treating an unresectable locally advanced or metastatic NSCLC comprises a NSCLC that is PD-L1 positive. In some embodiments, a method of treating an unresectable locally advanced or metastatic NSCLC comprises a NSCLC that is PD-1 positive. In some embodiments, a method of treating an unresectable locally advanced or metastatic NSCLC comprises a NSCLC that is PD-L1 positive in a subject, wherein said treating comprises second-line or third-line treatment. In some embodiments, a method of treating an unresectable locally advanced or metastatic NSCLC comprises a NSCLC that is PD-1 positive in a subject, wherein said treating comprises second-line or third-line treatment. In some embodiments, a method of treating an unresectable locally advanced or metastatic NSCLC comprises a squamous or a non-squamous NSCLC that is PD-L1 positive. In some embodiments, a method of treating an unresectable locally advanced or metastatic NSCLC comprises a squamous or a non-squamous NSCLC that is PD-1 positive.

In some embodiments, the method described herein comprises treating the primary cancer and secondary metastasis of the cancer. In some embodiments, the method described herein comprises treating the secondary metastasis of the cancer. In some embodiments, the method described herein is a first line treatment of the cancer. In some embodiments, the method described herein is a first line treatment of the cancer or a later line of therapy. In some embodiments, the method described herein is a second line treatment of the cancer. In some embodiments, the method described herein is a third line treatment of the cancer. In some embodiments, the method described herein is a fourth line treatment of the cancer. In some embodiments, the method described herein is a fifth line treatment of the cancer. In some embodiments, the method described herein is a sixth line treatment of the cancer. In some embodiments, the method described herein is a seventh line treatment of the cancer. In some embodiments, the method described herein is an eighth line treatment of the cancer. In some embodiments, the method described herein is a ninth line treatment of the cancer. In some embodiments, the method described herein comprises a second line treatment or a third line treatment of the cancer.

In some embodiments, the cancer comprises an unresectable locally advanced or metastatic cancer. In other embodiments, the cancer comprises an unresectable locally advanced or metastatic solid cancer. In some embodiments, the unresectable locally advanced or metastatic cancer comprises a melanoma, a renal cell carcinoma (RCC), a non-small cell lung cancer (NSCLC), a head and neck squamous cell carcinoma (HNSCC), gastric or gastro-esophageal cancer, esophageal squamous cell carcinoma, cutaneous squamous cell carcinoma (cSCC), pancreatic adenocarcinoma, cholangiocarcinoma (bile duct cancer), hepato-cellular carcinoma (HCC), colorectal cancer (CRC), epithelial ovarian cancer, cervical cancer, endometrial cancer, thyroid cancer having follicular or papillary histology, urothelial cancer, bladder cancer, uterine cancer, gallbladder cancer, or Merkel cell carcinoma.

In other embodiments, the cancer comprises an unresectable locally advanced or metastatic NSCLC. In other embodiments, the unresectable locally advanced or metastatic NSCLC comprises a PD-L1-positive NSCLC. In some embodiments, the unresectable locally advanced or metastatic NSCLC comprises squamous or non-squamous that is PD-L1-positive. In some embodiments, the tumor proportion score [TPS] of the NSCLC is greater than or equal to 1%. In some embodiments, the NSCLC does not harbor an activating EGFR mutation. In some embodiments, the NSCLC does not harbor an ALK rearrangement. In some embodiments the NSCLC does not harbor an activating EGFR mutation that has progressed during or following treatment with an anti-PDx (either PD-1 or PD-L1). In some embodiments the NSCLC does not harbor an ALK rearrangement that has progressed during or following treatment with an anti-PDx (either PD-1 or PD-L1). In some embodiments, the NSCLC progressed during or following treatment with an anti-PDx (either PD-1 or PD-L1) with platinum-based chemotherapy. In other embodiments, the NSCLC progressed during or following treatment with an anti-PDx (either PD-1 or PD-L1) without platinum-based chemotherapy.

In some embodiments, treating an unresectable locally advanced or metastatic NSCLC comprises treating the primary NSCLC and secondary metastasis of the NSCLC. In some embodiments, treating an unresectable locally advanced or metastatic NSCLC comprises treating the secondary metastasis of the NSCLC. In some embodiments, treating an unresectable locally advanced or metastatic NSCLC comprises a first line treatment of the NSCLC. In some embodiments, treating an unresectable locally advanced or metastatic NSCLC comprises a second line treatment of the NSCLC. In some embodiments, treating an unresectable locally advanced or metastatic NSCLC comprises a third line treatment of the NSCLC. In some embodiments, treating an unresectable locally advanced or metastatic NSCLC comprises a fourth line treatment of the NSCLC. In some embodiments, treating an unresectable locally advanced or metastatic NSCLC comprises a fifth line treatment of the NSCLC. In some embodiments, treating an unresectable locally advanced or metastatic NSCLC comprises a sixth line treatment of the NSCLC. In some embodiments, treating an unresectable locally advanced or metastatic NSCLC comprises a seventh line treatment of the NSCLC. In some embodiments, treating an unresectable locally advanced or metastatic NSCLC comprises an eighth line treatment of the NSCLC. In some embodiments, treating an unresectable locally advanced or metastatic NSCLC comprises a ninth line treatment of the NSCLC. In some embodiments, treating an unresectable locally advanced or metastatic NSCLC comprises second and third line treatments of the NSCLC.

In some embodiments, a “first line” treatment is the initial and preferred therapeutic option for a given medical condition or disease, chosen because it is considered the most effective and least risky based on evidence and clinical guidelines. In some embodiments, a “second line” treatment is a treatment that is given to a patient when the initial treatment (first-line therapy) is not effective or stops working. In some embodiments, a “third line” treatment is a treatment that is given to a patient when both initial treatment (first-line therapy) and subsequent treatment (second-line therapy) are not effective or stop working. Fourth line, fifth line, etc. treatments are similarly treatment that is given to a patient when prior treatments failed, e.g., were not effective or stopped working.

In some embodiments, the present disclosure provides a method of treating an NSCLC in a subject comprising administering to said subject an anti-IL-2 antibody or a pharmaceutical composition thereof and avelumab or a pharmaceutical composition thereof. In some embodiments, the present disclosure provides a method of treating an NSCLC in a subject comprising administering to said subject an anti-IL-2 antibody or a pharmaceutical composition thereof, a loading dose of low dose IL-2 or a pharmaceutical composition thereof, and avelumab or a pharmaceutical composition thereof.

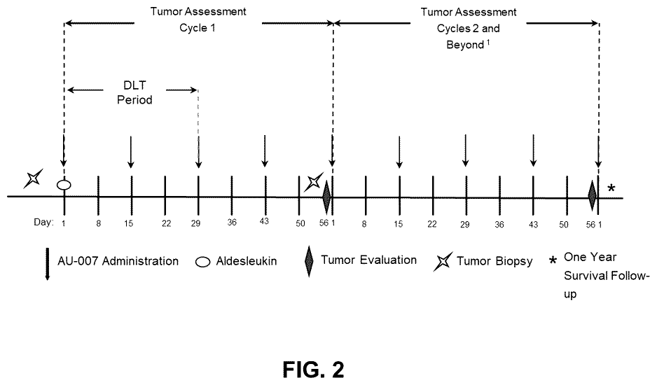

In some embodiments, multiple doses of anti-IL-2 antibody are administered (FIG. 2). In some embodiments, 2-50 doses of anti-IL-2 antibody are administered. In other embodiments, 20-50 doses of anti-IL-2 antibody are administered. In other embodiments, 10-40 doses of anti-IL-2 antibody are administered. In other embodiments, 20-30 doses of anti-IL-2 antibody are administered. In other embodiments, more than 20 doses of anti-IL-2 antibody are administered. In other embodiments, more than 30 doses of anti-IL-2 antibody are administered. In other embodiments, more than 40 doses of anti-IL-2 antibody are administered. In other embodiments, more than 50 doses of anti-IL-2 antibody are administered. In some embodiments, anti-IL-2 antibody doses are administered at regular intervals throughout the treatment. In some embodiments, the interval between anti-IL-2 antibody doses is once every 2 weeks, 4 weeks, 6 weeks, or 8 weeks. In some embodiments, the interval between anti-IL-2 antibody doses is 2 weeks.

In some embodiments, a single dose of avelumab is administered (FIG. 2). In some embodiments, multiple doses of avelumab are administered. In some embodiments, 2-50 doses of avelumab are administered. In other embodiments, 20-50 doses of avelumab are administered. In other embodiments, 10-40 doses of avelumab are administered. In other embodiments, 20-30 doses of avelumab are administered. In other embodiments, more than 20 doses of avelumab are administered. In other embodiments, more than 30 doses of avelumab are administered. In other embodiments, more than 40 doses of avelumab are administered. In other embodiments, more than 50 doses of avelumab are administered. In some embodiments, avelumab doses are administered at regular intervals throughout the treatment. In some embodiments, the interval between avelumab doses is once every 2 weeks, 4 weeks, 6 weeks, or 8 weeks. In some embodiments, the interval between avelumab doses is 2 weeks.

In some embodiments, a method as described herein comprises administering a loading dose of a low dose of IL-2 or a pharmaceutical composition thereof to the subject (FIG. 2). In some embodiments, the method comprises further administering one or more booster doses of a low dose of IL-2 or a pharmaceutical composition thereof to the subject. In some embodiments, the method comprises further administering a single booster dose of a low dose of IL-2 or a pharmaceutical composition thereof to the subject. In other embodiments, the method comprises further administering two or more booster doses of a low dose of IL-2 or a pharmaceutical composition thereof to the subject. In other embodiments, the method comprises further administering three or more booster doses of a low dose of IL-2 or a pharmaceutical composition thereof to the subject. In other embodiments, the method comprises further administering four or more booster doses of a low dose of IL-2 or a pharmaceutical composition thereof to the subject. In other embodiments, the method comprises further administering five or more booster doses of a low dose of IL-2 or a pharmaceutical composition thereof to the subject. In other embodiments, the method comprises administering a loading dose of low dose IL-2 and two, three, four, five, or six booster doses of low dose IL-2. In other embodiments, the method comprises administering a loading dose of low dose IL-2 and 6-10 or 10 or more booster doses of low dose IL-2. In some embodiments, low dose IL-2 boosters are administered at regular intervals throughout the treatment. In some embodiments, the interval between low dose IL-2 boosters is once every 2 weeks, 4 weeks, 6 weeks, or 8 weeks. In some embodiments, the interval between low dose IL-2 boosters is 2 weeks. In some embodiments, the interval between low dose IL-2 boosters is 8 weeks.

A skilled artisan would appreciate that duration of a treatment therapy, for example but not limited to for treating an unresectable locally advanced or metastatic NSCLC, may be based on the response or lack of response by the cancer to the treatment. In some embodiments, duration of a method of treating an unresectable locally advanced or metastatic NSCLC comprises about 2 years for patients who are receiving benefit from the treatment therapy. Benefit may in some embodiments be measured by tumor stabilization, tumor shrinkage, inhibiting or reducing growth of the tumor, inhibiting or reducing metastases of the tumor, inhibiting the production of new lesions, elimination of lesions, or any combination thereof. In some embodiments, duration of a method of treating an unresectable locally advanced or metastatic NSCLC comprises less than 2 years for patients who are receiving benefit from the treatment therapy. In some embodiments, duration of a method of treating an unresectable locally advanced or metastatic NSCLC comprises 1 year for patients who are receiving benefit from the treatment therapy. In some embodiments, duration of a method of treating an unresectable locally advanced or metastatic NSCLC comprises less than 1 years for patients who are receiving benefit from the treatment therapy.

In some embodiments of a method of treatment disclosed herein using a combination therapy, the administration of each components may be on a different treatment schedule for different durations (FIG. 2). For example but not limited to administration of IL-2 booster doses, wherein the number of booster doses administered may vary widely between patients. In some embodiments, a patient receives at least one additional IL-2 dose (a “booster” dose). In some embodiments, a patient receives at least 1, 2, 3, 4, 5, 6, 7, 8, 9, 10, 11, or 12 additional IL-2 doses (“booster” doses) over a 2 year period. In some embodiments, a patient receives less than 12 booster IL-2 doses over a 2 year period. In some embodiments, a patient receives 6 booster IL-2 doses over a 1 year period. In some embodiments, a patient receives less than 6 booster IL-2 doses over a 1 year period. In some embodiments, a patient receives 1, 2, 3, 4, 5, or 6 booster IL-2 doses over a 1 year period. In some embodiments, a patient receives 3 booster IL-2 doses over a 6 month period. In some embodiments, a patient receives less than 3 booster IL-2 doses over a 6 month period. In some embodiments, a patient receives 1, 2, or 3 booster IL-2 doses over a 6 month period.

In some embodiments, the IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:62, said HCDR2 comprises the amino acid sequence of SEQ ID NO:63, said HCDR3 comprises the amino acid sequence of SEQ ID NO:64, said LCDR1 comprises the amino acid sequence of SEQ ID NO:65, said LCDR2 comprises the amino acid sequence of DAS, and said LCDR3 comprises the amino acid sequence of SEQ ID NO:67.

In some embodiments, the loading dose of IL-2 is subcutaneously administered. In some embodiments, the booster dose of IL-2 is subcutaneously administered. In some embodiments, the loading dose of IL-2 is administered at a dose of between about 15,000 IU/kg of said subject's body weight—500,000 IU/kg of said subject's body weight. In some embodiments, the booster dose of IL-2 is administered at a dose of between about 15,000 IU/kg of said subject's body weight—500,000 IU/kg of said subject's body weight. In some embodiments, the booster dose of IL-2 is administered at a dose of 135,000 IU/kg of said subject's body weight.

In some embodiments of the methods described herein that comprise administering a booster dose of IL-2, the booster dose of IL-2 is administered to the subject with each treatment cycle, e.g., until objective tumor shrinkage, inhibiting or reducing growth of the tumor, inhibiting or reducing metastases of the tumor, inhibiting the production of new lesions, elimination of lesions, or any combination thereof, is observed on radiologic imaging or physical exam. In some embodiments, objective tumor shrinkage is observed on radiologic imaging, physical exam, or a combination thereof. In other embodiments, the booster dose of IL-2 is administered to the subject when the subject has one or more objective signs of worsening tumor growth kinetics. In some embodiments, a sign of worsening tumor growth kinetics comprises previously shrinking tumors becoming stable. In other embodiments, a sign of worsening tumor growth kinetics comprises new tumor growth. In other embodiments, a sign of worsening tumor growth kinetics comprises appearance of new tumor growth in a tumor that was previously stable or had decreased in size. In other embodiments, a sign of worsening tumor growth kinetics comprises an increase in tumor markers. In other embodiments, a sign of worsening tumor growth kinetics comprises appearance of metastases. In other embodiments, a sign of worsening tumor growth kinetics comprises any combination of the signs described herein. In some embodiments, a treatment cycle is 8 weeks (FIG. 2). In other embodiments, a treatment cycle is 2, 4, 6, 10, or 12 weeks.

A skilled artisan would appreciate that a “Target Lesion” is a measurable (measurable defined per the RECIST (Response Evaluation Criteria in Solid Tumors) rules) tumor lesion (locally advanced or metastatic) that's measurement is added to the measurements of up to 4 other Target Lesions and the total combined diameters of the Target Lesions is used to follow if the tumor is increasing or decreasing in size after each cycle of treatment. In certain embodiments, methods described herein inhibit or reduce metastases. This may be observed by the absence of new lesions. In some embodiments, methods described herein result in decrease or shrinkage in a target lesion. This may be observed by measurements showing the reduction in size of a tumor. In some embodiments, methods described herein result in the disappearance of target lesions (a tumor). This too may be observed by measurements showing the reduction in size and disappearance of a tumor.

In some embodiments, tumor evaluation comprises imaging the tumor to measure tumors and assess for any new tumors. A skilled clinician would know the best methods of imaging the tumor and patient for metastases. In some embodiments, imaging comprises computed tomography (CT) scan imaging. In some embodiments, imaging comprises magnetic resonance imaging (MRI). In some embodiments, imaging comprises positron emission tomography (PET) scan imaging. In some embodiments, imaging comprises ultrasound imaging. In some embodiments, imaging comprises CT scan imaging, MRI, PET scan imaging, or ultrasound imaging, or any combination thereof.

In some embodiments, tumor evaluation results in a decision to administer an IL-2 booster dose. In certain embodiments, an at least one booster dose is administered to said subject if tumor volume is stable, if one or more previously shrinking tumors becomes stable, if there is an increase in one or more tumor markers, if one or more new tumors are detected, if tumor growth is detected in one or more tumors that had previously been stable or had decreased in size, or any combination thereof.

In some embodiments, the administration of the one or more booster doses of IL-2 is prior to, concurrent with, or following the administration of said one or more additional doses of said anti-IL-2 antibody, one or more additional doses of said avelumab, or both.

In some embodiments, the anti-IL-2 antibody is administered at a dose of between about 0.5 mg/kg of a subject's body weight—12 mg/kg of said subject's body weight. In other embodiments, the anti-IL-2 antibody is administered at a dose of between about 4.5 mg/kg of a subject's body weight and 12 mg/kg of said subject's body weight. In other embodiments, the anti-IL-2 antibody is administered at a dose of between about 9 mg/kg of a subject's body weight and 12 mg/kg of said subject's body weight. In some embodiments, the anti-IL-2 antibody is administered at a dose of 9 mg/kg subject's body weight. In other embodiments, the anti-IL-2 antibody is administered at a dose of 12 mg/kg. In other embodiments, the anti-IL-2 antibody is administered at a dose of 6 mg/kg. In other embodiments, the anti-IL-2 antibody is administered at a dose of 3 mg/kg. In other embodiments, the anti-IL-2 antibody is administered at a dose of 10 mg/kg. In other embodiments, the anti-IL-2 antibody is administered at a dose of 15 mg/kg. In other embodiments, the anti-IL-2 antibody is administered at a dose of 20 mg/kg.

In some embodiments of a method of use of a combination therapy as described herein, the anti-IL-2 antibody is formulated for IV infusion.

In some embodiments, the methods described herein further comprise the step of administering one or more additional doses of the anti-IL-2 antibody. In other embodiments, the methods described herein further comprise the step of administering one or more additional doses of a pharmaceutical composition comprising the anti-IL-2 antibody.

In some embodiments, the anti-IL-2 antibody is administered on a bi-weekly (once every two weeks) schedule (FIG. 2). In other embodiments, the anti-IL-2 antibody is administered on a schedule as described herein above. In some embodiments, the anti-IL-2 antibody is administered weekly, bi-weekly (once every two weeks), once every three weeks, or once every four weeks.

In some embodiments, the schedule of administering the anti-IL-2 antibody may not be the same through the full course of the therapy, wherein administration may be bi-weekly for a time period and may then change to weekly, once every three weeks, once every four weeks, once every 5 weeks, or once every 6 weeks. Similarly, the schedule of administering the anti-IL-2 antibody may not be the same through the full course of the therapy, wherein administrations may be further apart, for example but not limited to once every 4-6 weeks and may then change to weekly, bi-weekly, or once every three weeks.

In some embodiments, the IL-2 is administered as a one-time loading dose and optionally as a booster dose as needed.

In some embodiments, the loading dose of IL-2 is administered at a dose of between about 15,000 IU/kg of said subject's body weight and 500,000 IU/kg of said subject's body weight. In other embodiments, the loading dose of IL-2 is administered at a dose of between about 45,000 and 270,000 IU/kg of the subject's body weight. In other embodiments, the loading dose of IL-2 is administered at a dose of between about 45,000 IU/kg and 135,000 IU/kg of said subject's body weight.

In some embodiments, the IL-2 loading dose is administered at a dose of 135,000 IU/kg of said subject's body weight. In other embodiments, the IL-2 loading dose is administered at a dose of 270,000 IU/kg. In other embodiments, the IL-2 loading dose is administered at a dose of 500,000 IU/kg. In other embodiments, the IL-2 loading dose is administered at a dose of 100,000 IU/kg. In other embodiments, the IL-2 loading dose is administered at a dose of 75,000 IU/kg. In other embodiments, the IL-2 loading dose is administered at a dose of 50,000 IU/kg.

In some embodiments, the booster dose of IL-2 is administered at a dose of between about 15,000 IU/kg of said subject's body weight and 500,000 IU/kg of said subject's body weight. In other embodiments, the booster dose of IL-2 is administered at a dose of between about 45,000 and 270,000 IU/kg of the subject's body weight. In other embodiments, the booster dose of IL-2 is administered at a dose of between about 45,000 IU/kg and 135,000 IU/kg of said subject's body weight.

In some embodiments, the IL-2 booster dose is administered at a dose of 135,000 IU/kg of said subject's body weight. In other embodiments, the IL-2 booster dose is administered at a dose of 270,000 IU/kg. In other embodiments, the IL-2 booster dose is administered at a dose of 500,000 IU/kg. In other embodiments, the IL-2 booster dose is administered at a dose of 100,000 IU/kg. In other embodiments, the IL-2 booster dose is administered at a dose of 75,000 IU/kg. In other embodiments, the IL-2 booster dose is administered at a dose of 50,000 IU/kg.

In some embodiments, the booster dose of IL-2 is administered once, twice, three times, four times, five times, or six times. In some embodiments, the booster dose of IL-2 is administered one week, two weeks, three weeks, four weeks, 5 weeks, 6 weeks, 7 weeks, or 8 weeks after the loading dose of IL-2. In some embodiments, the 2^{nd} booster dose of IL-2 is administered one week, two weeks, three weeks, four weeks, 5 weeks, 6 weeks, 7 weeks, or 8 weeks after the 1^{st} booster dose of IL-2. In some embodiments, a booster dose of IL-2 is administered one week, two weeks, three weeks, four weeks, 5 weeks, 6 weeks, 7 weeks, or 8 weeks after the prior dose of IL-2 to the subject.

In some embodiments, the anti-IL-2 antibody or composition thereof and the low dose IL-2 (loading or booster dose) or composition thereof are administered at a frequency or interval that is independent of one another.

In some embodiments, methods as described herein comprising the administration of an anti-IL-2 antibody or composition thereof and a low dose of IL-2 or composition thereof, the administration of the anti-IL-2 antibody or composition thereof and the administration of the low dose of IL-2 or composition thereof is concurrent. In other embodiments, the anti-IL-2 antibody or composition thereof is administered prior to the administration of the low dose of IL-2 or composition thereof. In other embodiments, the anti-IL-2 antibody or composition thereof is administered following the administration of the low dose of IL-2 or composition thereof.

In some embodiments, methods as described herein comprising the administration of an anti-IL-2 antibody or composition thereof and a loading dose of IL-2 or composition thereof, the administration of the anti-IL-2 antibody or composition thereof and the administration of the loading dose of IL-2 or composition thereof is concurrent. In other embodiments, the anti-IL-2 antibody or composition thereof is administered prior to the administration of the loading dose of IL-2 or composition thereof. In other embodiments, the anti-IL-2 antibody or composition thereof is administered following the administration of the loading dose of IL-2 or composition thereof.

In some embodiments, methods as described herein comprising the administration of an anti-IL-2 antibody or composition thereof and a booster dose of IL-2 or composition thereof, the administration of the anti-IL-2 antibody or composition thereof and the administration of the booster dose of IL-2 or composition thereof is concurrent. In other embodiments, the anti-IL-2 antibody or composition thereof is administered prior to the administration of the booster dose of IL-2 or composition thereof. In other embodiments, the anti-IL-2 antibody or composition thereof is administered following the administration of the booster dose of IL-2 or composition thereof.

In some embodiments, methods as described herein comprising the administration of an anti-IL-2 antibody or pharmaceutical composition thereof, a loading dose of IL-2 or pharmaceutical composition thereof, and an immune checkpoint inhibitor or a pharmaceutical composition thereof, wherein the administration of the anti-IL-2 antibody or pharmaceutical composition thereof, the administration of the loading dose of IL-2 or the pharmaceutical composition thereof, and or the administration of the immune checkpoint inhibitor or the pharmaceutical composition thereof is concurrent. In other embodiments, the anti-IL-2 antibody or composition thereof is administered prior to the administration of the loading dose of IL-2 or composition thereof and or prior to the administration of the immune checkpoint inhibitor. In other embodiments, the anti-IL-2 antibody or composition thereof is administered following the administration of the loading dose of IL-2 or composition thereof and or the administration of the immune checkpoint inhibitor. In some embodiments, the immune checkpoint inhibitor is a PD-L1 immune checkpoint inhibitor. In some embodiments, the immune checkpoint inhibitor is a PD-1 immune checkpoint inhibitor. In some embodiments, the immune checkpoint inhibitor is avelumab.

In some embodiments, methods as described herein comprising the administration of an immune checkpoint inhibitor comprise the step of administering the immune checkpoint inhibitor or a pharmaceutical composition thereof prior to the administration of the anti-IL-2 antibody. In some embodiments, the immune checkpoint inhibitor is administered concurrent with the administration of the anti-IL-2 antibody. In some embodiments, the immune checkpoint inhibitor is administered following the administration of the anti-IL-2 antibody. In some embodiments, the immune checkpoint inhibitor is a PD-L1 immune checkpoint inhibitor. In some embodiments, the immune checkpoint inhibitor is a PD-1 immune checkpoint inhibitor. In some embodiments, the immune checkpoint inhibitor is avelumab.

In some embodiments, methods as described herein comprising the administration of an immune checkpoint inhibitor comprise the step of administering the immune checkpoint inhibitor or a pharmaceutical composition thereof prior to the administration of the low dose of IL-2. In some embodiments, the immune checkpoint inhibitor is administered concurrent with the administration of the low dose of IL-2. In some embodiments, the immune checkpoint inhibitor is administered following the administration of the low dose of IL-2. In some embodiments, the low dose of IL-2 is the loading dose of IL-2. In other embodiments, the low dose of IL-2 is the booster dose of IL-2. In some embodiments, the immune checkpoint inhibitor is a PD-L1 immune checkpoint inhibitor. In some embodiments, the immune checkpoint inhibitor is a PD-1 immune checkpoint inhibitor. In some embodiments, the immune checkpoint inhibitor is avelumab.

In some embodiments, methods as described herein comprising the administration of avelumab comprise the step of administering avelumab or a pharmaceutical composition thereof prior to the administration of the anti-IL-2 antibody. In some embodiments, avelumab is administered concurrent with the administration of the anti-IL-2 antibody. In some embodiments, avelumab is administered following the administration of the anti-IL-2 antibody.

In some embodiments, methods as described herein comprising the administration of avelumab comprise the step of administering avelumab or a pharmaceutical composition thereof prior to the administration of the low dose of IL-2. In some embodiments, avelumab is administered concurrent with the administration of the low dose of IL-2. In some embodiments, avelumab is administered following the administration of the low dose of IL-2.

In some embodiments, a method of use of a combination therapy as described herein comprises administering an immune checkpoint inhibitor by IV infusion. In some embodiments, a method of use of a combination therapy as described herein comprises administering an immune checkpoint inhibitor at a dose of 800 mg. In some embodiments, a method of use of a combination therapy as described herein comprises administering an immune checkpoint inhibitor at a dose of 800 mg by IV infusion. In some embodiments of a method of use of a combination therapy, the immune checkpoint inhibitor comprises avelumab.

In some embodiments, a method of use of a combination therapy as described herein comprises administering avelumab by IV infusion. In some embodiments, a method of use of a combination therapy comprises administering avelumab at a dose of 400 mg. In some embodiments, a method of use of a combination therapy comprises administering avelumab at a dose of 600 mg. In some embodiments, a method of use of a combination therapy comprises administering avelumab at a dose of 800 mg. In some embodiments, a method of use of a combination therapy comprises administering avelumab at a dose of 1000 mg. In some embodiments, a method of use of a combination therapy comprises administering avelumab at a dose of 1200 mg. In some embodiments, a method of use of a combination therapy comprises administering avelumab at a dose of 1400 mg. In some embodiments, a method of use of a combination therapy comprises administering avelumab at a dose of 1600 mg. In some embodiments, a method of use of a combination therapy comprises administering avelumab at a dose of 360-1600 mg. In some embodiments, a method of use of a combination therapy comprises administering avelumab at a dose of 600-1000 mg.

In some embodiments, a method of use of a combination therapy comprises administering avelumab at a dose of 400 mg by IV infusion. In some embodiments, a method of use of a combination therapy comprises administering avelumab at a dose of 600 mg by IV infusion. In some embodiments, a method of use of a combination therapy comprises administering avelumab at a dose of 800 mg by IV infusion. In some embodiments, a method of use of a combination therapy comprises administering avelumab at a dose of 1000 mg by IV infusion. In some embodiments, a method of use of a combination therapy comprises administering avelumab at a dose of 1200 mg by IV infusion. In some embodiments, a method of use of a combination therapy comprises administering avelumab at a dose of 1400 mg by IV infusion. In some embodiments, a method of use of a combination therapy comprises administering avelumab at a dose of 1600 mg by IV infusion. In some embodiments, a method of use of a combination therapy comprises administering avelumab at a dose of 360-1600 mg by IV infusion. In some embodiments, a method of use of a combination therapy comprises administering avelumab at a dose of 600-1000 mg by IV infusion.

In some embodiments, a method of use of a combination therapy comprises administering avelumab at a dose of 10 mg/kg of a subject's body weight. In some embodiment, a method of use of a combination therapy comprises administering avelumab at a dose of 10 mg/kg of a subject's body weight for subjects who weigh between 70-80 kg.

In some embodiments, methods as described herein comprising the administration of a PD-L1 immune checkpoint inhibitor comprise the step of administering the PD-L1 immune checkpoint inhibitor or a pharmaceutical composition thereof prior to the administration of the anti-IL-2 antibody. In some embodiments, the PD-L1 immune checkpoint inhibitor is administered concurrent with the administration of the anti-IL-2 antibody. In some embodiments, the PD-L1 immune checkpoint inhibitor is administered following the administration of the anti-IL-2 antibody.

In some embodiments, methods as described herein comprising the administration of the PD-L1 immune checkpoint inhibitor comprise the step of administering the PD-L1 immune checkpoint inhibitor or a pharmaceutical composition thereof prior to the administration of the low dose of IL-2. In some embodiments, the PD-L1 immune checkpoint inhibitor is administered concurrent with the administration of the low dose of IL-2. In some embodiments, the PD-L1 immune checkpoint inhibitor is administered following the administration of the low dose of IL-2.

In some embodiments, methods as described herein comprising the administration of a PD-1 immune checkpoint inhibitor comprise the step of administering the PD-1 immune checkpoint inhibitor or a pharmaceutical composition thereof prior to the administration of the anti-IL-2 antibody. In some embodiments, the PD-1 immune checkpoint inhibitor is administered concurrent with the administration of the anti-IL-2 antibody. In some embodiments, the PD-1 immune checkpoint inhibitor is administered following the administration of the anti-IL-2 antibody.

In some embodiments, methods as described herein comprising the administration of the PD-1 immune checkpoint inhibitor comprise the step of administering the PD-1 immune checkpoint inhibitor or a pharmaceutical composition thereof prior to the administration of the low dose of IL-2. In some embodiments, the PD-1 immune checkpoint inhibitor is administered concurrent with the administration of the low dose of IL-2. In some embodiments, the PD-1 immune checkpoint inhibitor is administered following the administration of the low dose of IL-2.

In some embodiments, the methods described herein further comprise the step of administering one or more additional doses of avelumab. In other embodiments, the methods described herein further comprise the step of administering one or more additional doses of a pharmaceutical composition comprising avelumab.

In some embodiments, the avelumab is administered at least once every week, bi-weekly (once every two weeks), once every three weeks, once every four weeks, once every five weeks, or at least once every 6 weeks. In some embodiments, the schedule of administering the avelumab may not be the same through the full course of the therapy, wherein administration may be bi-weekly for a time period and may then change to weekly, once every three weeks, once every four weeks, once every 5 weeks, or once every 6 weeks. Similarly, the schedule of administering the avelumab may not be the same through the full course of the therapy, wherein administrations may be further apart, for example but not limited to once every 4 to 6 weeks and may then change to weekly, bi-weekly (once every two weeks), or once every three weeks. In some embodiments of a method of use of a combination therapy disclosed herein, administration of avelumab to said subject is once every two weeks (FIG. 2).

In some embodiments of a method of use of a combination therapy described herein, the anti-IL-2 antibody is administered once every two weeks, a loading dose of IL-2 is administered, and the avelumab is administered once every two weeks. In certain embodiments, the method further comprises administering a IL-2 booster once every 8 weeks if analysis of one or more objective signs of worsening tumor growth kinetics indicates the booster is warranted. In some embodiments, one or more objective signs comprises previously shrinking tumors becoming stable, increase in tumor markers, tumor growth, or appearance of new tumor growth in a tumor that had previously been stable or had decreased in size, or any combination thereof. In some embodiments, with each cycle of treatment an analysis of one or more objective signs are performed, wherein a skilled clinician determines if an IL-2 booster dose should be administered as part of the method.

In certain embodiments, disclosed herein is a method of treating an unresectable locally advanced or metastatic non-small cell lung cancer (NSCLC) that is PD-L1 positive in a subject, said method comprising administering to said subject an anti-IL-2 antibody at a dose of 9 mg/kg of said subject's body weight or a pharmaceutical composition thereof, a loading dose of 135,000 IU/kg of said subject's body weight of IL-2 or a pharmaceutical composition thereof, and avelumab at a dose of 800 mg or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:62, said HCDR2 comprises the amino acid sequence of SEQ ID NO: 63, said HCDR3 comprises the amino acid sequence of SEQ ID NO:64, said LCDR1 comprises the amino acid sequence of SEQ ID NO:65, said LCDR2 comprises the amino acid sequence of DAS, and said LCDR3 comprises the amino acid sequence of SEQ ID NO: 67; wherein said NSCLC comprises unresectable locally advanced or metastatic NSCLC that is PD-L1 positive; wherein said treating comprises second-line or third-line treatment; wherein said pharmaceutical composition(s) further comprises a pharmaceutically acceptable carrier, thereby treating said NSCLC in said subject.

In certain embodiments, disclosed herein is a method of treating an unresectable locally advanced or metastatic non-small cell lung cancer (NSCLC) that is PD-1 positive in a subject, said method comprising administering to said subject an anti-IL-2 antibody at a dose of 9 mg/kg of said subject's body weight or a pharmaceutical composition thereof, a loading dose of 135,000 IU/kg of said subject's body weight of IL-2 or a pharmaceutical composition thereof, and avelumab at a dose of 800 mg or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:62, said HCDR2 comprises the amino acid sequence of SEQ ID NO: 63, said HCDR3 comprises the amino acid sequence of SEQ ID NO:64, said LCDR1 comprises the amino acid sequence of SEQ ID NO:65, said LCDR2 comprises the amino acid sequence of DAS, and said LCDR3 comprises the amino acid sequence of SEQ ID NO: 67; wherein said NSCLC comprises unresectable locally advanced or metastatic NSCLC that is PD-1 positive; wherein said treating comprises second-line or third-line treatment; wherein said pharmaceutical composition(s) further comprises a pharmaceutically acceptable carrier, thereby treating said NSCLC in said subject.

In some embodiments, the anti-IL-2 antibodies described and exemplified herein, bind the portion of IL-2 that interacts with the alpha (CD25) receptor subunit that is a component of the IL-2 trimeric receptor (CD25/CD132/CD122, sometimes represented as α/β/γ) found on Treg cells, eosinophils, and pulmonary and vascular endothelial cells. In some embodiments, the anti-IL-2 antibodies disclosed herein prevent activation of the trimeric IL-2 receptor found on Tregs, eosinophils, and pulmonary and vascular endothelial cells. In some embodiments, the anti-IL-2 antibodies disclosed herein bind to IL-2, activate signaling through the IL-2 dimer receptor (CD132/CD122, sometimes represented as β/γ) found on naïve Teff cells, NK cells, and Natural killer T (NKT) cells. Thus, in some embodiments, the present disclosure provides a method of preventing activation of the trimeric IL-2 receptor found on Tregs, eosinophils, and pulmonary and vascular endothelial cells. In other embodiments, the present disclosure provides a method of activating signaling through the IL-2 dimer receptor (CD132/CD122, sometimes represented as β/γ) found on naïve Teff cells, NK cells, and Natural killer T (NKT) cells.

In some embodiments, administration of an anti-IL-2 antibody disclosed herein, for example but not limited to AU-007, provides a method of preventing activation of the trimeric IL-2 receptor found on Tregs, eosinophils, and pulmonary and vascular endothelial cells and provides a method of activating signaling through the IL-2 dimer receptor (CD132/CD122, sometimes represented as β/γ) found on naïve Teff cells, NK cells, and Natural killer T (NKT) cells. A skilled artisan would appreciate that the anti-IL-2 antibody may function as an immunomodulator. Immunomodulators may either increase or decrease immune function. In some embodiments, an anti-IL-2 antibody described herein, for example AU-007, increases immune function. In other embodiments, an anti-IL-2 antibody described herein, for example AU-007, decreases immune functions. In some embodiments, an anti-IL-2 antibody, for example AU-007 may either increase or decrease immune function.

A complex of IL-2 and anti-IL-2 antibodies induced proliferation of memory phenotype effector T cells (MP) CD8⁺ cells and NK cells, while there was a much smaller effect on CD4⁺ Tregs. Thus, the engineered anti-IL-2 antibodies disclosed herein would be useful in adjusting immune cell populations and inducing differential expansion of certain immune effector cells. In some embodiments, such differential expansion of immune effect cells would result in robust activation of the immune system and could be useful for treatment of tumors. Thus, in some embodiments, the present disclosure provides a method of inducing proliferation of memory phenotype effector T cells (MP) CD8⁺ cells and NK cells. In other embodiments, the present disclosure provides a method of activating the immune system of a subject having an NSCLC.

In certain embodiments, the present disclosure provides a method of treating a cancer that progressed after prior checkpoint inhibitor therapy. In some embodiments, the present disclosure provides a method of treating a subject having cancer, wherein said subject was previously treated with an immune checkpoint inhibitor. In other embodiments, the subject being treated has a cancer that is recalcitrant to treatment with one or more immune checkpoint inhibitors.

In certain embodiments, the present disclosure provides a method of treating a cancer that progressed after prior PD-1 therapy. In some embodiments, the present disclosure provides a method of treating a subject having cancer, wherein said subject was previously treated with a PD-1 inhibitor. In other embodiments, the subject being treated has a cancer that is recalcitrant to treatment with one or more PD-1 inhibitors.

In certain embodiments, the present disclosure provides a method of treating a cancer that progressed after prior PD-L1 therapy. In some embodiments, the present disclosure provides a method of treating a subject having cancer, wherein said subject was previously treated with a PD-L1 inhibitor. In other embodiments, the subject being treated has a cancer that is recalcitrant to treatment with one or more PD-L1 inhibitors.

In some embodiments, a method as described herein further comprises administering to said subject an adjuvant treatment. In other embodiments, a method as described herein further comprises administering to said subject a neoadjuvant treatment. In other embodiments, a method as described herein further comprises administering to said subject an adjuvant treatment and a neoadjuvant treatment.

In some embodiments, an “adjuvant” treatment is administered after primary treatment, such as surgery, to eliminate residual cancer cells that may remain and reduce the risk of the cancer returning. In some embodiments, a “neoadjuvant” treatment is administered before the primary treatment, typically surgery, to shrink tumors, make surgery less invasive, treat micrometastases, and improve the overall success of cancer treatment. In some embodiments, the adjuvant or neo-adjuvant treatment comprises chemotherapy, radiation therapy, hormone therapy, targeted therapy, immunotherapy, or any combination thereof.

In some embodiments, the present disclosure provides a method for reducing the size of the NSCLC. In other embodiments, the present disclosure provides a method for inhibiting the growth of the NSCLC. In other embodiments, the present disclosure provides a method for reducing the growth of the NSCLC. In other embodiments, the present disclosure provides a method for inhibiting the metastases of the NSCLC. In other embodiments, the present disclosure provides a method for reducing the metastases of the NSCLC. In other embodiments, the present disclosure provides a method for increasing the time before detection of a new lesion. In other embodiments, the present disclosure provides a method for decreasing a target lesion. In other embodiments, the present disclosure provides a method for shrinking a target lesion. In other embodiments, the present disclosure provides a method for obliterating or eradicating a target lesion. In other embodiments, the present disclosure provides a method for removing a target lesion. In other embodiments, the present disclosure provides a method for causing a target lesion to disappear.

In some embodiments, treatment of an unresectable locally advanced or metastatic NSCLC in a subject reduces the size of the NSCLC, inhibits or reduces growth of the NSCLC, or inhibits or reduces metastases of said NSCLC, or any combination thereof.

In certain embodiments of a method of use of a combination therapy disclosed herein, the method comprises (i) reducing the size of the tumor, (ii) inhibiting or reducing growth of the tumor, (iii) inhibiting or reducing metastases of said tumor, (iv) inhibiting the production of new lesions, (v) decreasing or shrinking target lesions, (vi) eliminating target lesions, or (vii) any combination thereof.

In some embodiments, the method comprises the step of administering an anti-IL-2 antibody. In other embodiments, the method comprises the step of administering an anti-IL-2 antibody and a low loading dose of IL-2. In other embodiments, the method comprises administering an anti-IL-2 antibody and one or more immune checkpoint inhibitors. In other embodiments, the method comprises administering an anti-IL-2 antibody and avelumab. In other embodiments, the method comprises administering an anti-IL-2 antibody, a low loading dose of IL-2 and one or more immune checkpoint inhibitors. In other embodiments, the method comprises administering an anti-IL-2 antibody, a low loading dose of IL-2, and avelumab. In other embodiments, the method comprises administering an anti-IL-2 antibody, a low loading dose of IL-2, one or more low booster doses of IL-2, and one or more immune checkpoint inhibitors. In other embodiments, the method comprises administering an anti-IL-2 antibody, a low loading dose of IL-2, one or more low booster doses of IL-2, and avelumab.

As used throughout, the terms “cancer” and “tumor” may in some embodiments be used interchangeably having the same meanings and qualities.

In other embodiments, the present disclosure provides a method of treating an unresectable NSCLC. In other embodiments, the present disclosure provides a method of treating a locally advanced NSCLC. In other embodiments, the present disclosure provides a method of treating an unresectable locally advanced NSCLC. In some embodiments, the present disclosure provides a method of treating a metastatic NSCLC.

In other embodiments, a subject treated by a method disclosed herein has an unresectable NSCLC. In other embodiments, a subject treated by a method disclosed herein has a locally advanced NSCLC. In other embodiments, a subject treated by a method disclosed herein has an unresectable locally advanced NSCLC. In some embodiments, a subject treated by a method disclosed herein has a metastatic NSCLC.

In some embodiments, an unresectable tumor is a tumor that is not considered a candidate for surgical removal due to factors like advanced stage, metastasis, or being too large, too close to vital structures, or too difficult to access. In some embodiments, an unresectable tumor is an inoperable tumor having a technical or anatomical barrier to resection, such as complete encasement of a major blood vessel, that makes complete surgical removal uncertain or impossible.

In some embodiments, the NSCLC comprises an immune sensitive NSCLC.

In some embodiments, treatment of an unresectable locally advanced or metastatic NSCLC in a subject as described herein comprises maintenance treatments. In some embodiments, maintenance treatments are administered to maintain the absence of an unresectable locally advanced or metastatic NSCLC. In some embodiments, maintenance treatments are administered to maintain lack of metastasis of an unresectable locally advanced or metastatic NSCLC. In some embodiments, maintenance treatments are administered to inhibit metastasis of an unresectable locally advanced or metastatic NSCLC. In some embodiments, maintenance treatments are administered to maintain lack of growth of an unresectable locally advanced or metastatic NSCLC. In some embodiments, maintenance treatments are administered to inhibit growth of an unresectable locally advanced or metastatic NSCLC.

In some embodiments, treatment of NSCLC comprises prophylactic treatment of, for example, but not limited to, a subject harboring a genetic marker or markers with a high risk of developing NSCLC.

In some embodiments, the present disclosure provides a method of promoting differential growth of immune cells in a subject, comprising the step of preparing a composition comprising an anti-IL-2 antibody disclosed herein, and administering the composition to the subject, thereby promoting differential growth of immune cells in the subject. In some embodiments, the present disclosure provides a method of promoting differential growth of immune cells in a subject, comprising the step of preparing a composition comprising IL-2 and the anti-IL-2 antibody disclosed herein, and administering the composition to the subject, thereby promoting differential growth of immune cells in the subject. In some embodiments, the subject can be an animal or a human. In some embodiments, the immune cells can be CD8⁺ cells or NK cells.

In some embodiments, disclosed herein is a method of treating an unresectable locally advanced or metastatic NSCLC in a subject, comprising the step of administering to the subject a composition comprising an anti-IL-2 antibody as disclosed herein, wherein said antibody promotes differential growth of subsets of immune cells and decreases undesirable effects caused by IL-2, thereby treating unresectable locally advanced or metastatic NSCLC in said subject. In some embodiments, a method of treating a disease disclosed here comprises use of a composition comprising an anti-IL-2 antibody and IL-2. In some embodiments, a method of treating a disease comprises treating an unresectable locally advanced or metastatic NSCLC. In some embodiments, a method of treating a condition comprises treating a weak immune system and the treatment prophylactically boosts the immune system.

In some embodiments, methods of promoting differential growth of immune cells in a subject, comprise the step of preparing and administering a composition comprising an anti-IL-2 antibody disclosed herein. In some embodiments, methods of promoting differential growth of immune cells in a subject, comprise the step of preparing and administering a composition comprising IL-2. In some embodiments, methods of promoting differential growth of immune cells in a subject, comprise the step of preparing and administering a composition comprising an immune checkpoint inhibitor. In some embodiments, methods of promoting differential growth of immune cells in a subject, comprise the step of preparing and administering a composition comprising avelumab. In some embodiments, methods of promoting differential growth of immune cells in a subject, comprise the step of preparing and administering an anti-IL-2 antibody and IL-2 or composition(s) thereof. In some embodiments, the IL-2 is a loading dose of IL-2. In some embodiments, the IL-2 is a booster dose of IL-2. In some embodiments, the loading dose or booster dose or both of IL-2 is low dose IL-2.

In some embodiments, methods of promoting differential growth of immune cells in a subject, comprise the step of preparing and administering an anti-IL-2 antibody and an immune checkpoint inhibitor or composition(s) thereof. In other embodiments, methods of promoting differential growth of immune cells in a subject, comprise the step of preparing and administering an anti-IL-2 antibody and a PD-L1 inhibitor or a PD-1 inhibitor or composition(s) thereof. In other embodiments, methods of promoting differential growth of immune cells in a subject, comprise the step of preparing and administering an anti-IL-2 antibody and avelumab or composition(s) thereof. In other embodiments, the method comprises preparing and administering an anti-IL-2 antibody, an immune checkpoint inhibitor, and IL-2 or composition(s) thereof. In other embodiments, the method comprises preparing and administering an anti-IL-2 antibody, a PD-L1 inhibitor or a PD-1 inhibitor, and IL-2 or composition(s) thereof. In other embodiments, the method comprises preparing and administering an anti-IL-2 antibody, avelumab, and IL-2 or composition(s) thereof. In some embodiments, the IL-2 is a loading dose of IL-2. In some embodiments, the IL-2 is a booster dose of IL-2. In some embodiments, the loading dose or booster dose or both of IL-2 is low dose IL-2.

In some embodiments, the methods described herein comprise the steps of:
(a) preparing a composition comprising an anti-IL-2 antibody as disclosed herein; and
(b) administering the composition from (a) to the subject.

In some embodiments, the methods described herein comprise the steps of:
(a) preparing a composition comprising an anti-IL-2 antibody as disclosed herein;
(b) preparing a composition comprising IL-2 as disclosed herein;
(c) preparing a composition comprising an immune checkpoint inhibitor as disclosed herein; and
(d) administering the composition from (a), (b), and (c) as disclosed herein, to the subject. In some embodiments, the immune checkpoint inhibitor comprises a PD-L1 checkpoint inhibitor. In other embodiments, the immune checkpoint inhibitor comprises a PD-1 checkpoint inhibitor. In some embodiments, the immune checkpoint inhibitor is avelumab.

In some embodiments, the methods described herein comprise the steps of:
(a) preparing a composition comprising an anti-IL-2 antibody as disclosed herein;
(b) preparing a composition comprising a loading dose of IL-2 as disclosed herein;
(c) preparing a composition comprising an immune checkpoint inhibitor as disclosed herein; and
(d) administering the composition from (a), (b), and (c) as disclosed herein, to the subject, wherein additional doses of the anti-IL-2 antibody are administered for the duration of the treatment, and additional doses of the immune checkpoint inhibitor are administered for the duration of the treatment, and if analysis of objective signs warrants, an at least one booster IL-2 dose is administered. In some embodiments, the immune checkpoint inhibitor comprises a PD-L1 checkpoint inhibitor. In other embodiments, the immune checkpoint inhibitor comprises a PD-1 checkpoint inhibitor. In some embodiments, the immune checkpoint inhibitor is avelumab.

In some embodiments, the methods described herein comprise the steps of:
(a) preparing a composition comprising an anti-IL-2 antibody as disclosed herein;
(b) preparing a composition comprising a loading dose of IL-2 as disclosed herein;
(c) preparing a composition comprising avelumab as disclosed herein; and
(d) administering the composition from (a), (b), and (c) as disclosed herein, to the subject, wherein additional doses of the anti-IL-2 antibody are administered for the duration of the treatment, and additional doses of avelumab are administered for the duration of the treatment, and if analysis of objective signs warrants, an at least one booster IL-2 dose is administered.

In some embodiments, the subject can be an animal or a human.

In some embodiments, treatment with the anti-IL-2 antibodies expands immune cells comprising one or more of naïve T cells, memory T cells, CD8⁺ T cells, NK cells, and Natural Killer T cells. In some embodiments, treatment with the anti-IL-2 antibodies decreases one or more undesirable effects caused by IL-2 such as activation of regulatory T cells, apoptosis of CD25⁺ T effector cells, pulmonary edema, pneumonia, and IL-2-induced vascular leakage.

In some embodiments, an anti-IL-2 antibody as described herein is engineered or modified. In some embodiments, the engineered or modified anti-IL-2 antibodies comprise a heavy chain variable region and a light chain variable region having the sequences of SEQ ID NO: 26 and SEQ ID NO:27.

In another embodiment, the engineered or modified anti-IL-2 antibodies comprise a heavy chain variable region having complementarity determining region (CDR) 1, CDR2 and CDR3. In some embodiments, the heavy chain CDR1, CDR2 and CDR3 comprise amino acid sequences of SEQ ID NOs: 62-64, respectively.

In other embodiments, the engineered or modified anti-IL-2 antibodies comprise a light chain variable region having complementarity determining region (CDR) 1, CDR2 and CDR3. In some embodiments, the light chain CDR1, CDR2 and CDR3 comprise amino acid sequences of SEQ ID NO:65, DAS, and SEQ ID NO: 67 respectively.

In other embodiments, the amino acid sequence of the full length heavy chain of the anti-IL-2 antibody is as set forth in SEQ ID NO: 72 and the amino acid sequence of the full length light chain of the anti-IL-2 antibody is as set forth in SEQ ID NO: 73. In some embodiments, the engineered anti-IL-2 antibody is an IgG. In other embodiments, the engineered anti-IL-2 antibody is an IgA. In other embodiments, the engineered anti-IL-2 antibody is an IgM.

In other embodiments, the engineered anti-IL-2 antibody is an IgE. In other embodiments, the engineered anti-IL-2 antibody is an IgD. In other embodiments, the engineered anti-IL-2 antibody is a Fv. In other embodiments, the engineered anti-IL-2 antibody is a scFv. In other embodiments, the engineered anti-IL-2 antibody is a Fab. In other embodiments, the engineered anti-IL-2 antibody is a F(ab′) 2. In some embodiments, the IgG is of the subclass of IgG1. In other embodiments, the IgG is of the subclass of IgG2. In other embodiments, the IgG is of the subclass of IgG3. In other embodiments, the IgG is of the subclass of IgG4. In some embodiments, the engineered antibody is part of a minibody. In other embodiments, the engineered antibody is part of a diabody. In other embodiments, the engineered antibody is part of a triabody antibody.

In some embodiments, the engineered anti-IL-2 antibody comprises a heavy chain comprising a mutation that reduces binding to a fragment crystallizable gamma receptor (FcγR; Fcγ receptor). In some embodiments, the mutation comprises L234A, L235A mutations.

In some embodiments, the polypeptides disclosed herein may be administered to a subject directly, or by administering to the subject a nucleic acid sequence encoding the polypeptides. In some embodiments, the nucleic acid sequence may be carried by a vector.

In some embodiments, a polynucleotide sequence encoding an engineered anti-IL-2 antibody is used in a method of treating a subject with a disease or condition as described herein, wherein the polynucleotide encodes an antibody comprising a heavy chain variable region having the amino acid sequence of SEQ ID NO:26 In some embodiments, a polynucleotide sequence encoding an engineered anti-IL-2 antibody is used in a method of treating a subject with a disease or condition as described herein, wherein the polynucleotide encodes an antibody comprising a light chain variable region having the amino acid sequence of SEQ ID NO:27. In some embodiments, a polynucleotide sequence encoding an engineered anti-IL-2 antibody is used in a method of treating a subject with a disease or condition as described herein, wherein the polynucleotide encodes an antibody comprising a heavy chain variable region and a light chain variable region having the amino acid sequences of one of SEQ ID NOs: 26 and 27.

In some embodiments of a method of using a polynucleotide to treat a disease or condition as described above, the polynucleotide encodes an engineered anti-IL-2 antibody that can be an IgG, IgA, IgM, IgE, IgD, a Fv, a scFv, a Fab, or a F(ab′)₂. The IgG can be of the subclass of IgG1, IgG2, IgG3, or IgG4. In some embodiments, the polynucleotide encodes an engineered antibody which is part of a minibody, a diabody, or a triabody antibody.

In some embodiments a polynucleotide sequence encoding an engineered anti-IL-2 antibody is used in a method of treating a subject with a disease or condition as described herein, wherein the polynucleotide sequence comprises the sequence of SEQ ID NO: 35.

In some embodiments of a method of treating a disease or condition as described herein, the immune effector cells that are activated by the treatment are CD8+ cells or NK cells. In some embodiments, the anti-IL-2 antibodies disclosed herein, or a complex of IL-2 and the anti-IL-2 antibodies disclosed herein, exhibits pronounced effect in inducing proliferation of MP CD8⁺ cells and NK cells, while there was much smaller effect on CD4⁺ Tregs. In certain embodiments, there is no effect on CD4⁺ Tregs.

In certain embodiments, methods of use of an anti-IL-2 antibody disclosed herein provide a pro-stimulatory effect. In some embodiments, said use comprises the anti-IL-2 antibody. In some embodiments, said use comprises the anti-IL-2 antibody and an IL-2 (loading or booster dose), a PD-1 inhibitor, a PD-L1 inhibitor, avelumab, or a combination thereof.

Thus, the engineered anti-IL-2 antibodies disclosed herein are useful in adjusting immune cell populations and inducing differential expansion of certain immune effector cells in a method of treating a disease such as an unresectable locally advanced or metastatic NSCLC, or treating a condition such as IL-2 induced pulmonary edema, or IL-2-induced vascular leakage.

In some embodiments, a method disclosed herein comprising administering an anti-IL-2 antibody or a pharmaceutical composition thereof, a loading dose of IL-2 or a pharmaceutical composition thereof, and avelumab or a pharmaceutical composition thereof to a subject further comprises administering an additional one or more immune checkpoint inhibitors targeting one or more immune checkpoints to the subject. In some embodiments, a subject is treated with said immune checkpoint inhibitors concurrently, before, or after treatment with an anti-IL-2 antibody. In some embodiments, a subject is treated with said immune checkpoint inhibitors concurrently, before, or after treatment with low dose IL-2, which in some embodiments, is a loading does and in other embodiments, is a booster dose. In some embodiments, a subject is treated with said immune checkpoint inhibitors concurrently, before, or after treatment with avelumab. In some embodiments of a method of treating disclosed herein, an immune checkpoint comprises a Programmed death-ligand 1 (PD-L1), a Programmed cell death protein 1 (PD-1), a Cytotoxic T-lymphocyte protein 4 (CTLA-4), a T-cell immunoreceptor with Ig and ITIM domains (TIGIT), a Metalloproteinase inhibitor 3 (TIM-3), B7 homolog 3 (B7-H3), a cluster of differentiation 73 (CD73), a Lymphocyte-activation gene 3 (LAG3), a cluster of differentiation 27 (CD27), a cluster of differentiation 70 (CD70), a Tumor necrosis factor ligand superfamily member 9 (4-1BB), a Glucocorticoid-Induced TNFR-Related (GITR), a Tumor necrosis factor receptor superfamily member 4 (OX40), a cluster of differentiation 47 (SIRP-alpha (CD47)), a cluster of differentiation 39 (CD39), an Immunoglobulin Like Domain Containing Receptor 2 (ILDR2), a V-Domain Ig Suppressor Of T Cell Activation (VISTA), a B and T lymphocyte attenuator (BTLA), or a V-set domain containing T cell activation inhibitor 1 (VTCN-1), or a combination thereof.

In some embodiments, the one or more immune checkpoint inhibitors comprises avelumab, atezolizumab, durvalumab, sugemalimab, cosibelimab, envafolimab, nivolumab, pembrolizumab, cemiplimab, camrelizumab, zimberelimab, tislelizumab, sintilimab, teriprizumab, prolgolimab, penpulimab, dostarlimab, genolimzumab, retifanlimab, or a combination thereof. In some embodiments, the PD-L1 immune checkpoint inhibitor comprises avelumab, atezolizumab, durvalumab, sugemalimab, cosibelimab or envafolimab. In some embodiments, the PD-1 immune checkpoint inhibitor comprises nivolumab, pembrolizumab, cemiplimab, camrelizumab, zimberelimab, tislelizumab, sintilimab, teriprizumab, prolgolimab, penpulimab, dostarlimab, genolimzumab, or retifanlimab.

In other embodiments, the checkpoint inhibitor comprises atezolizumab. In other embodiments, the checkpoint inhibitor comprises durvalumab. In other embodiments, the checkpoint inhibitor comprises sugemalimab. In other embodiments, the checkpoint inhibitor comprises or envafolimab. In some embodiments, the checkpoint inhibitor comprises cosibelimab. In other embodiments, the checkpoint inhibitor comprises nivolumab. In other embodiments, the checkpoint inhibitor comprises pembrolizumab. In other embodiments, the checkpoint inhibitor comprises cemiplimab. In other embodiments, the checkpoint inhibitor comprises camrelizumab. In other embodiments, the checkpoint inhibitor comprises zimberelimab. In other embodiments, the checkpoint inhibitor comprises tislelizumab. In other embodiments, the checkpoint inhibitor comprises sintilimab. In other embodiments, the checkpoint inhibitor comprises teriprizumab. In other embodiments, the checkpoint inhibitor comprises prolgolimab. In other embodiments, the checkpoint inhibitor comprises penpulimab. In other embodiments, the checkpoint inhibitor comprises dostarlimab. In other embodiments, the checkpoint inhibitor comprises genolimzumab. In other embodiments, the checkpoint inhibitor comprises retifanlimab.

In some embodiments, the anti-IL-2 antibody, the low dose IL-2 (loading or booster dose) and the immune checkpoint inhibitor are comprised in different pharmaceutical compositions.

In some embodiments, the immune checkpoint inhibitor is administered prior to the anti-IL-2 antibody administration. In other embodiments, the immune checkpoint inhibitor is administered concurrent with the anti-IL-2 antibody administration. In other embodiments, the immune checkpoint inhibitor is administered following the administration of the anti-IL-2 antibody.

In some embodiments, the immune checkpoint inhibitor is administered prior to the low dose IL-2 administration. In other embodiments, the immune checkpoint inhibitor is administered concurrently with the low dose IL-2 administration. In other embodiments, the immune checkpoint inhibitor is administered following the administration of the low dose IL-2.

As discussed above, in some embodiments, a therapeutic method of treatment as disclosed herein, further comprises administering an additional active agent comprising a checkpoint inhibitor. One skilled in the art would appreciate that a combination therapy comprising an anti-IL-2 antibody therapy in the presence or absence of IL-2, and additionally comprising a checkpoint inhibitor may utilize any of the therapeutic compositions or formulations comprising an anti-IL-2 antibody+/−IL-2, and a checkpoint inhibitor as provided herein. In some embodiments, at least two checkpoint inhibitors are used in a combination therapy.

In some embodiments of a method of use of a combination therapy as described herein, a subject comprises a mammalian subject. In some embodiments, a subject comprises a human subject. In some embodiments, a subject suffers from immune deficiency. Treatment of an immune deficient subject would, in some embodiments, comprise a prophylactic treatment.

### Combination Therapies

In some embodiments, an anti-IL-2 antibody or composition thereof as disclosed herein, is used as part of a combination therapy. In some embodiments, an anti-IL-2 antibody or composition thereof as disclosed herein, is used in combination with an immune checkpoint inhibitor. In some embodiments, an anti-IL-2 antibody or composition thereof as disclosed herein, is used in combination with IL-2. In some embodiments, an anti-IL-2 antibody or composition thereof as disclosed herein, is used in combination with IL-2 and with an immune checkpoint inhibitor. In some embodiments, the IL-2 is low-dose IL-2. In some embodiments, the IL-2 is a loading dose of IL-2. In some embodiments, the IL-2 is a booster dose of IL-2. In some embodiments, IL-2 is administered as a loading dose and as a booster dose at a later time period depending on the need of the patient.

In some embodiments, an anti-IL-2 antibody or a composition thereof, is used in combination with an immune checkpoint inhibitor. In some embodiments, the term “immune checkpoint inhibitor” may encompass any compound or molecule capable of inhibiting the function of a checkpoint protein. In some embodiments, the term “immune checkpoint inhibitor” may encompass any compound or molecule which targets immune checkpoint proteins. An artisan would appreciate that “immune checkpoints” are key regulators of the immune system that when stimulated can dampen the immune response to an immunologic stimulus. Checkpoint inhibitors can block inhibitory checkpoints, and thereby restore immune system function. In some embodiments, the one or more checkpoint inhibitors comprise immune checkpoint inhibitors.

A skilled artisan would appreciate that the terms “immune checkpoint inhibitors” (ICIs), “checkpoint inhibitors,” and the like may be used interchangeably herein having all the same qualities and meanings, wherein an immune checkpoint inhibitor encompasses compounds that inhibit the activity or control mechanism(s) of the immune system. Immune system checkpoints, or immune checkpoints, are inhibitory pathways in the immune system that generally act to maintain self-tolerance or modulate the duration and amplitude of physiological immune responses to minimize collateral tissue damage. Checkpoint inhibitors can inhibit an immune system checkpoint by inhibiting the activity of a protein in the pathway. Immune checkpoint inhibitor targets include, but are not limited to PD-1, PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, and VTCN-1. In some embodiments, an anti-IL-2 antibody therapy is used in combination with an immune checkpoint inhibitor, wherein the target of the immune checkpoint inhibitor comprises PD-1, PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1, or any combination thereof.

Checkpoint inhibitors may include antibodies, or antigen binding fragments thereof, other binding proteins, biologic therapeutics, or small molecules, that bind to and block or inhibit the activity of one or more of PD-1, PD-L1, CTLA-4, TIGIT, TIM-3, B7-H3, CD73, LAG3, CD27, CD70, 4-1BB, GITR, OX40, SIRP-alpha (CD47), CD39, ILDR2, VISTA, BTLA, or VTCN-1. Illustrative checkpoint inhibitors include but are not limited to those listed in Table 1 below.

TABLE 1
Non-Limiting Examples of Checkpoint Inhibitors
and the Immune Checkpoint Inhibitor Target.

| Drug name | ICI Target |
|---|---|
| Avelumab | anti-PD-L1 |
| Atezolizumab | anti-PD-L1 |
| Durvalumab | anti-PD-L1 |
| Sugemalimab | anti-PD-L1 |
| Envafolimab | anti-PD-L1 |
| Nivolumab (Opdivo)-BMS | anti-PD-1 |
| Pembrolizumab(keytruda) | anti-PD-1 |
| Cemiplimab | anti-PD-1 |
| Camrelizumab | anti-PD-1 |
| Zimberelimab | anti-PD-1 |
| Tislelizumab | anti-PD-1 |
| Sintilimab | anti-PD-1 |
| Teriprizumab | anti-PD-1 |
| Prolgolimab | anti-PD-1 |
| Penpulimab | anti-PD-1 |
| Dostarlimab | anti-PD-1 |
| Genolimzumab | anti-PD-1 |
| Retifanlimab | anti-PD-1 |
| Ipilimumab (Yervoy) | anti-CTLA4 |
| Tiragolumab | anti-TIGIT |
| Domvanalimab | anti-TIGIT |
| Vibostolimab | anti-TIGIT |
| BMS-986207 | anti-TIGIT |
| EOS-448 | anti-TIGIT |
| COM-902 | anti-TIGIT |
| Sabatolimab | anti-TIM3 |
| Cobolimab | anti-TIM3 |
| BMS-986258 | anti-TIM3 |
| INCAGN-02390 | anti-TIM3 |
| S-95018 | anti-TIM3 |
| Omburtamab | anti-B7-H3 |
| MGC-018 | anti-B7-H3 |
| Enoblituzumab | anti-B7-H3 |
| Oleclumab | anti-CD73 |
| BMS-986179 | anti-CD73 |
| NZV-930 | anti-CD73 |
| CPX-006 | anti-CD73 |
| MK-4280 | anti-LAG3 |
| Sym-022 | anti-LAG3 |
| Ieramilimab | anti-LAG3 |
| BI-754111 | anti-LAG3 |
| MK-5890 | anti-CD27 |
| Varlilumab | anti-CD27 |
| Cusatuzumab | anti-CD70 |
| Vorsetuzumab | anti-CD70 |
| Urelumab | anti-4-1BB (agonist) |
| Utomilumab | anti-4-1BB (agonist) |
| ATOR-1017 | anti-4-1BB (agonist) |
| RO-7122290 | anti-4-1BB (agonist) |
| INCAGN-01876 | anti-GITR (agonist) |
| BMS-986156 | anti-GITR (agonist) |
| TRX-518 | anti-GITR (agonist) |
| GWN-323 | anti-GITR (agonist) |
| BMS-986178 | anti-OX40 (agonist) |
| INCAGN-1949 | anti-OX40 (agonist) |
| GSK-3174998 | anti-OX40 (agonist) |
| BGB-A-445 | anti-OX40 (agonist) |
| BI-765063 | anti- SIRP-alpha (CD47) |
| ALX-148 | SIRP-alpha (CD47) |
| IPH-52 | anti-CD39 |
| TTX-030 | anti-CD39 |
| BAY-1905254 | anti-ILDR2 |
| Onvatilimab | anti-VISTA |
| K01401-020 | anti-VISTA |
| JS-004 | anti-BTLA |
| FPA-150 | anti-VTCN1 |

In some embodiments, the checkpoint inhibitor comprises a PD-1 inhibitor. In some embodiments, the checkpoint inhibitor comprises a PD-L1 inhibitor. In some embodiments, the checkpoint inhibitor comprises a CTLA-4 inhibitor. In some embodiments, the checkpoint inhibitor comprises a TIGIT inhibitor. In some embodiments, the checkpoint inhibitor comprises a TIM-3 inhibitor. In some embodiments, the checkpoint inhibitor comprises a B7-H3 inhibitor. In some embodiments, the checkpoint inhibitor comprises a CD73 inhibitor. In some embodiments, the checkpoint inhibitor comprises a LAG3 inhibitor. In some embodiments, the checkpoint inhibitor comprises a CD27 inhibitor. In some embodiments, the checkpoint inhibitor comprises a CD70 inhibitor. In some embodiments, the checkpoint inhibitor comprises a 4-1BB agonist binder. In some embodiments, the checkpoint inhibitor comprises a GITR agonist binder. In some embodiments, the checkpoint inhibitor comprises a OX40 agonist binder. In some embodiments, the checkpoint inhibitor comprises a SIRP-alpha (CD47) inhibitor. In some embodiments, the checkpoint inhibitor comprises a CD39 inhibitor. In some embodiments, the checkpoint inhibitor comprises a ILDR2 inhibitor. In some embodiments, the checkpoint inhibitor comprises a VISTA inhibitor. In some embodiments, the checkpoint inhibitor comprises a BTLA inhibitor. In some embodiments, the checkpoint inhibitor comprises a VTCN-1 inhibitor.

In some embodiments, the checkpoint inhibitor comprises a combination of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, a VTCN-1 inhibitor. In some embodiments, the checkpoint inhibitor comprises at least two checkpoint inhibitors selected from of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, and a VTCN-1 inhibitor.

In some embodiments, a pharmaceutical composition for use in a combination therapy, as described herein, comprises an effective amount of a checkpoint inhibitor, as described herein, and a pharmaceutically acceptable carrier.

In some embodiments, a composition disclosed herein comprises a checkpoint inhibitor and a pharmaceutically acceptable carrier. In some embodiments, a composition disclosed herein comprises a combination of checkpoint inhibitors, and a pharmaceutically acceptable carrier. In some embodiments, a composition comprises a checkpoint inhibitor comprising a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, a VTCN-1 inhibitor. In some embodiments, the checkpoint inhibitor comprises at least two checkpoint inhibitors selected from of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, or a VTCN-1 inhibitor, and a pharmaceutically acceptable carrier. In some embodiments, a composition comprises at least two checkpoint inhibitors selected from a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, a VTCN-1 inhibitor. In some embodiments, the checkpoint inhibitor comprises at least two checkpoint inhibitors selected from of a PD-1 inhibitor, a PD-L1 inhibitor, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, and a VTCN-1 inhibitor, and a pharmaceutically acceptable carrier.

In certain embodiments, when more than one checkpoint inhibitor is used in a therapeutic method described herein, each checkpoint inhibitor is comprised within a separate composition. In certain embodiments, when more than one checkpoint inhibitor is used in a therapeutic method described herein, checkpoint inhibitor may be comprised within the same composition.

In some embodiments, a combination therapy comprises use of an anti-IL-2 antibody or composition thereof as described herein, and a checkpoint inhibitor or a composition thereof. In some embodiments, a combination therapy comprises use of an anti-IL-2 antibody or composition thereof and IL-2 as described herein, and a checkpoint inhibitor or a composition thereof.

As used herein, in some embodiments the terms “combination” and “combination therapy” may be used interchangeably having all the same meanings and qualities.

In other embodiments, a combination of the present disclosure comprises an anti-IL-2 antibody or composition thereof as described herein, and a checkpoint inhibitor or a composition thereof. In some embodiments, a combination of the present disclosure comprises an anti-IL-2 antibody or composition thereof and IL-2 or a composition thereof as described herein, and a checkpoint inhibitor or a composition thereof.

In some embodiments, a combination of the present disclosure comprises an anti-IL-2 antibody or composition thereof and low dose of IL-2 or composition thereof as described herein, and a checkpoint inhibitor or a composition thereof. In some embodiments, a combination of the present disclosure comprises an anti-IL-2 antibody or composition thereof and low dose loading dose (low loading dose) of IL-2 or composition thereof as described herein, and a checkpoint inhibitor or a composition thereof. In some embodiments, a combination of the present disclosure comprises an anti-IL-2 antibody or composition thereof and a low dose booster dose (low booster dose) of IL-2 or composition thereof as described herein, and a checkpoint inhibitor or a composition thereof.

In some embodiments, a combination therapy as described herein comprises an anti-IL-2 antibody formulated for IV infusion and administration at a dose of 9 mg/kg. In other embodiments, the anti-IL-2 antibody is formulated for IV infusion and administration at a dose of between about 0.5 mg/kg of a subject's body weight and 12 mg/kg of said subject's body weight. In other embodiments, the anti-IL-2 antibody is formulated for IV infusion and administration at a dose of between about 4.5 mg/kg of a subject's body weight and 12 mg/kg of said subject's body weight. In other embodiments, the anti-IL-2 antibody is formulated for IV infusion and administration at a dose of between about 9 mg/kg of a subject's body weight and 12 mg/kg of said subject's body weight. In some embodiments, a combination therapy as described herein comprises an anti-IL-2 antibody formulated for intravenous (IV) administration.

In some embodiments, a combination therapy as described herein comprises an anti-IL-2 antibody formulated for administration at a dose of 9 mg/kg. In other embodiments, the anti-IL-2 antibody is formulated administration at a dose of between about 0.5 mg/kg of a subject's body weight and 12 mg/kg of said subject's body weight. In other embodiments, the anti-IL-2 antibody is formulated for administration at a dose of between about 4.5 mg/kg of a subject's body weight and 12 mg/kg of said subject's body weight. In other embodiments, the anti-IL-2 antibody is formulated for administration at a dose of between about 9 mg/kg of a subject's body weight and 12 mg/kg of said subject's body weight. In some embodiments, a combination therapy as described herein comprises an anti-IL-2 antibody formulated for intravenous (IV) administration.

In some embodiments, a combination therapy as described herein comprises a loading low dose of IL-2 at a dose of 135,000 IU/kg. In some embodiments, a combination therapy as described herein comprises a booster dose of IL-2 at a dose of 135,000 IU/kg. In some embodiments, the loading or booster IL-2 dose is formulated for subcutaneous injection. In some embodiments, the loading or booster dose is formulated for subcutaneous injection and administration at a dose of between about 45,000 IU/kg of a subject's body weight and 270,000 IU/kg of said subject's body weight. In other embodiments, the loading or booster dose is formulated for subcutaneous injection and administration at a dose of between about 45,000 IU/kg of a subject's body weight and 135,000 IU/kg of said subject's body weight. In other embodiments, the loading or booster dose is formulated for subcutaneous injection and administration at a dose of between 15,000 IU/kg of said subject's body weight 500,000 IU/kg of said subject's body weight. In some embodiments, a combination therapy as described herein comprises a low dose of IL-2 formulated for subcutaneous injection and administration at 135,000 IU/kg of said subject's body weight.

In some embodiments, a combination therapy as described herein comprises the immune checkpoint inhibitor formulated for IV infusion and administration at a dose of 800 mg. In some embodiments, a combination therapy as described herein comprises avelumab formulated, e.g., for IV infusion, and administration at a dose of 800 mg. In some embodiments, a combination therapy as described herein comprises an immune checkpoint inhibitor formulated, e.g., for IV infusion, and administration. In some embodiments, a combination therapy as described herein comprises avelumab formulated, e.g., for IV infusion and administration. In some embodiments, the immune checkpoint inhibitor comprises avelumab.

In some embodiments, a combination therapy as described herein comprises avelumab formulated for IV infusion and administration at a dose of 400 mg. In some embodiments, a combination therapy as described herein comprises avelumab formulated for IV infusion and administration at a dose of 600 mg. In some embodiments, a combination therapy as described herein comprises avelumab formulated for IV infusion and administration at a dose of 800 mg. In some embodiments, a combination therapy as described herein comprises avelumab formulated for IV infusion and administration at a dose of 1000 mg. In some embodiments, a combination therapy as described herein comprises avelumab formulated for IV infusion and administration at a dose of 1200 mg. In some embodiments, a combination therapy as described herein comprises avelumab formulated for IV infusion and administration at a dose of 1400 mg. In some embodiments, a combination therapy as described herein comprises avelumab formulated for IV infusion and administration at a dose of 1600 mg. In some embodiments, a combination therapy as described herein comprises avelumab formulated for IV infusion and administration at a dose of 360-1600 mg. In some embodiments, a combination therapy as described herein comprises avelumab formulated for IV infusion and administration at a dose of 600-1000 mg.

In certain embodiments, disclosed herein is a combination therapy comprising an anti-IL-2 antibody or a pharmaceutical composition thereof, a loading dose of IL-2 or a pharmaceutical composition thereof, and avelumab or a pharmaceutical composition thereof, wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3, wherein said HCDR1 comprises the amino acid sequence of SEQ ID NO:62, said HCDR2 comprises the amino acid sequence of SEQ ID NO: 63, said HCDR3 comprises the amino acid sequence of SEQ ID NO:64, said LCDR1 comprises the amino acid sequence of SEQ ID NO:65, said LCDR2 comprises the amino acid sequence of DAS, and said LCDR3 comprises the amino acid sequence of SEQ ID NO: 67; wherein said anti-IL-2 antibody is formulated for administration at a dose of 9 mg/kg of a subject's body weight, the loading dose of IL-2 is formulated for subcutaneous administration at a dose of 135,000 IU/kg of a subject's body weight, and said avelumab is formulated for administration at a dose of 800 mg; and wherein said pharmaceutical composition(s) further comprises a pharmaceutically acceptable carrier. In some embodiments, the combination therapy may be used for treating unresectable locally advanced or metastatic solid cancer. In some embodiments, the combination therapy may be used for treating unresectable locally advanced or metastatic non-small cell lung cancer (NSCLC). In some embodiments, the combination therapy may be used for treating unresectable locally advanced or metastatic non-small cell lung cancer (NSCLC) that is PD-L1 positive. In some embodiments, the combination therapy may be used for treating unresectable locally advanced or metastatic non-small cell lung cancer (NSCLC) that is PD-L1 positive, wherein treatment consists of 2nd line or 3rd line treatment. In other embodiments, the combination therapy may be used for treating unresectable locally advanced or metastatic non-small cell lung cancer (NSCLC) that is PD-1 positive. In other embodiments, the combination therapy may be used for treating unresectable locally advanced or metastatic non-small cell lung cancer (NSCLC) that is PD-1 positive, wherein treatment consists of 2nd line or 3rd line treatment.

In some embodiments, a combination therapy may include an immune checkpoint inhibitor targeting PD-1 or PD-L1. In some embodiments the immune checkpoint inhibitor targeting PD-1 comprises any of nivolumab, pembrolizumab, cemiplimab, camrelizumab, zimberelimab, tislelizumab, sintilimab, teriprizumab, prolgolimab, penpulimab, dostarlimab, genolimzumab, or retifanlimab. In some embodiments the immune checkpoint inhibitor targeting PD-L1 comprises any of avelumab, atezolizumab, durvalumab, sugemalimab, envafolimab, or cosibelimab.

In some embodiments, a combination therapy as described herein comprises pembrolizumab formulated for IV infusion and administration at a dose of 300 mg. In some embodiments, a combination therapy as described herein comprises pembrolizumab formulated for IV infusion and administration at a dose of 600 mg. In some embodiments, a combination therapy as described herein comprises pembrolizumab formulated for IV infusion and administration at a dose of 2 mg/kg of the subject's body weight. In some embodiments, a combination therapy as described herein comprises pembrolizumab formulated for IV administration. In some embodiments, the immune checkpoint inhibitor comprises pembrolizumab.

In some embodiments, a combination therapy as described herein comprises pembrolizumab formulated for IV infusion and administration at a dose of 150 mg. In some embodiments, a combination therapy as described herein comprises pembrolizumab formulated for IV infusion and administration at a dose of 75 mg. In some embodiments, a combination therapy as described herein comprises pembrolizumab formulated for IV infusion and administration at a dose of 450 mg. In some embodiments, a combination therapy as described herein comprises pembrolizumab formulated for IV infusion and administration at a dose of 750 mg. In some embodiments, a combination therapy as described herein comprises pembrolizumab formulated for IV infusion and administration at a dose of 150-750 mg. In some embodiments, a combination therapy as described herein comprises pembrolizumab formulated for IV infusion and administration at a dose of 300-600 mg.

In some embodiments, a combination therapy as described herein comprises cemiplimab formulated for IV infusion and administration at a dose of 350 mg. In some embodiments, a combination therapy as described herein comprises cemiplimab formulated for IV administration. In some embodiments, the immune checkpoint inhibitor comprises cemiplimab.

In some embodiments, a combination therapy as described herein comprises cemiplimab formulated for IV infusion and administration at a dose of 150 mg. In some embodiments, a combination therapy as described herein comprises cemiplimab formulated for IV infusion and administration at a dose of 225 mg. In some embodiments, a combination therapy as described herein comprises cemiplimab formulated for IV infusion and administration at a dose of 475 mg. In some embodiments, a combination therapy as described herein comprises cemiplimab formulated for IV infusion and administration at a dose of 600 mg. In some embodiments, a combination therapy as described herein comprises cemiplimab formulated for IV infusion and administration at a dose of 150-600 mg. In some embodiments, a combination therapy as described herein comprises cemiplimab formulated for IV infusion and administration at a dose of 350-400 mg.

In some embodiments, a combination therapy as described herein comprises camrelizumab formulated for IV infusion and administration at a dose of 200 mg. In some embodiments, a combination therapy as described herein comprises camrelizumab 30 formulated for IV administration. In some embodiments, the immune checkpoint inhibitor comprises camrelizumab.

In some embodiments, a combination therapy as described herein comprises camrelizumab formulated for IV infusion and administration at a dose of 100 mg. In some embodiments, a combination therapy as described herein comprises camrelizumab formulated for IV infusion and administration at a dose of 150 mg. In some embodiments, a combination therapy as described herein comprises camrelizumab formulated for IV infusion and administration at a dose of 300 mg. In some embodiments, a combination therapy as described herein comprises camrelizumab formulated for IV infusion and administration at a dose of 400 mg. In some embodiments, a combination therapy as described herein comprises camrelizumab formulated for IV infusion and administration at a dose of 100-400 mg. In some embodiments, a combination therapy as described herein comprises camrelizumab formulated for IV infusion and administration at a dose of 200-300 mg.

In some embodiments, a combination therapy as described herein comprises zimberelimab formulated for IV infusion and administration at a dose of 240 mg. In some embodiments, a combination therapy as described herein comprises zimberelimab formulated for IV administration. In some embodiments, the immune checkpoint inhibitor comprises zimberelimab.

In some embodiments, a combination therapy as described herein comprises zimberelimab formulated for IV infusion and administration at a dose of 100 mg. In some embodiments, a combination therapy as described herein comprises zimberelimab formulated for IV infusion and administration at a dose of 175 mg. In some embodiments, a combination therapy as described herein comprises zimberelimab formulated for IV infusion and administration at a dose of 350 mg. In some embodiments, a combination therapy as described herein comprises zimberelimab formulated for IV infusion and administration at a dose of 500 mg. In some embodiments, a combination therapy as described herein comprises zimberelimab formulated for IV infusion and administration at a dose of 100-500 mg. In some embodiments, a combination therapy as described herein comprises zimberelimab formulated for IV infusion and administration at a dose of 240-300 mg.

In some embodiments, a combination therapy as described herein comprises tislelizumab formulated for IV infusion and administration at a dose of 150 mg. In some embodiments, a combination therapy as described herein comprises tislelizumab formulated for IV infusion and administration at a dose of 200 mg. In some embodiments, a combination therapy as described herein comprises tislelizumab formulated for IV infusion and administration at a dose of 300 mg. In some embodiments, a combination therapy as described herein comprises tislelizumab formulated for IV administration. In some embodiments, the immune checkpoint inhibitor comprises tislelizumab.

In some embodiments, a combination therapy as described herein comprises tislelizumab formulated for IV infusion and administration at a dose of 100 mg. In some embodiments, a combination therapy as described herein comprises tislelizumab formulated for IV infusion and administration at a dose of 400 mg. In some embodiments, a combination therapy as described herein comprises tislelizumab formulated for IV infusion and administration at a dose of 100-400 mg. In some embodiments, a combination therapy as described herein comprises tislelizumab formulated for IV infusion and administration at a dose of 150-300 mg.

In some embodiments, a combination therapy as described herein comprises sintilimab formulated for IV infusion and administration at a dose of 200 mg. In some embodiments, a combination therapy as described herein comprises sintilimab formulated for IV administration. In some embodiments, the immune checkpoint inhibitor comprises sintilimab.

In some embodiments, a combination therapy as described herein comprises sintilimab formulated for IV infusion and administration at a dose of 100 mg. In some embodiments, a combination therapy as described herein comprises sintilimab formulated for IV infusion and administration at a dose of 150 mg. In some embodiments, a combination therapy as described herein comprises sintilimab formulated for IV infusion and administration at a dose of 300 mg. In some embodiments, a combination therapy as described herein comprises sintilimab formulated for IV infusion and administration at a dose of 400 mg. In some embodiments, a combination therapy as described herein comprises sintilimab formulated for IV infusion and administration at a dose of 100-400 mg. In some embodiments, a combination therapy as described herein comprises sintilimab formulated for IV infusion and administration at a dose of 200-300 mg.

In some embodiments, a combination therapy as described herein comprises teriprizumab formulated for IV infusion and administration at a dose of 200 mg. In some embodiments, a combination therapy as described herein comprises teriprizumab formulated for IV administration. In some embodiments, the immune checkpoint inhibitor comprises teriprizumab.

In some embodiments, a combination therapy as described herein comprises teriprizumab formulated for IV infusion and administration at a dose of 100 mg. In some embodiments, a combination therapy as described herein comprises teriprizumab formulated for IV infusion and administration at a dose of 150 mg. In some embodiments, a combination therapy as described herein comprises teriprizumab formulated for IV infusion and administration at a dose of 300 mg. In some embodiments, a combination therapy as described herein comprises teriprizumab formulated for IV infusion and administration at a dose of 400 mg. In some embodiments, a combination therapy as described herein comprises teriprizumab formulated for IV infusion and administration at a dose of 100-400 mg. In some embodiments, a combination therapy as described herein comprises teriprizumab formulated for IV infusion and administration at a dose of 200-300 mg.

In some embodiments, a combination therapy as described herein comprises prolgolimab formulated for IV infusion and administration at a dose of 250 mg. In some embodiments, a combination therapy as described herein comprises prolgolimab formulated for IV administration. In some embodiments, the immune checkpoint inhibitor comprises prolgolimab.

In some embodiments, a combination therapy as described herein comprises prolgolimab formulated for IV infusion and administration at a dose of 100 mg. In some embodiments, a combination therapy as described herein comprises prolgolimab formulated for IV infusion and administration at a dose of 150 mg. In some embodiments, a combination therapy as described herein comprises prolgolimab formulated for IV infusion and administration at a dose of 350 mg. In some embodiments, a combination therapy as described herein comprises prolgolimab formulated for IV infusion and administration at a dose of 500 mg. In some embodiments, a combination therapy as described herein comprises prolgolimab formulated for IV infusion and administration at a dose of 100-500 mg. In some embodiments, a combination therapy as described herein comprises prolgolimab formulated for IV infusion and administration at a dose of 250-300 mg.

In some embodiments, a combination therapy as described herein comprises penpulimab formulated for IV infusion and administration at a dose of 200 mg. In some embodiments, a combination therapy as described herein comprises penpulimab formulated for IV administration. In some embodiments, the immune checkpoint inhibitor comprises penpulimab.

In some embodiments, a combination therapy as described herein comprises penpulimab formulated for IV infusion and administration at a dose of 100 mg. In some embodiments, a combination therapy as described herein comprises penpulimab formulated for IV infusion and administration at a dose of 150 mg. In some embodiments, a combination therapy as described herein comprises penpulimab formulated for IV infusion and administration at a dose of 300 mg. In some embodiments, a combination therapy as described herein comprises penpulimab formulated for IV infusion and administration at a dose of 400 mg. In some embodiments, a combination therapy as described herein comprises penpulimab formulated for IV infusion and administration at a dose of 100-400 mg. In some embodiments, a combination therapy as described herein comprises penpulimab formulated for IV infusion and administration at a dose of 200-300 mg.

In some embodiments, a combination therapy as described herein comprises dostarlimab formulated for IV infusion and administration at a dose of 500 mg. In some embodiments, a combination therapy as described herein comprises dostarlimab formulated for IV infusion and administration at a dose of 1000 mg. In some embodiments, a combination therapy as described herein comprises dostarlimab formulated for IV administration. In some embodiments, the immune checkpoint inhibitor comprises dostarlimab.

In some embodiments, a combination therapy as described herein comprises dostarlimab formulated for IV infusion and administration at a dose of 125 mg. In some embodiments, a combination therapy as described herein comprises dostarlimab formulated for IV infusion and administration at a dose of 250 mg. In some embodiments, a combination therapy as described herein comprises dostarlimab formulated for IV infusion and administration at a dose of 750 mg. In some embodiments, a combination therapy as described herein comprises dostarlimab formulated for IV infusion and administration at a dose of 1500 mg. In some embodiments, a combination therapy as described herein comprises dostarlimab formulated for IV infusion and administration at a dose of 125-1500 mg. In some embodiments, a combination therapy as described herein comprises dostarlimab formulated for IV infusion and administration at a dose of 500-10000 mg.

In some embodiments, a combination therapy as described herein comprises genolimzumab formulated for IV infusion and administration at a dose of 150 mg. In some embodiments, a combination therapy as described herein comprises genolimzumab formulated for IV infusion and administration at a dose of 300 mg. In some embodiments, a combination therapy as described herein comprises genolimzumab formulated for IV administration. In some embodiments, the immune checkpoint inhibitor comprises genolimzumab.

In some embodiments, a combination therapy as described herein comprises genolimzumab formulated for IV infusion and administration at a dose of 50 mg. In some embodiments, a combination therapy as described herein comprises genolimzumab formulated for IV infusion and administration at a dose of 100 mg. In some embodiments, a combination therapy as described herein comprises genolimzumab formulated for IV infusion and administration at a dose of 400 mg. In some embodiments, a combination therapy as described herein comprises genolimzumab formulated for IV infusion and administration at a dose of 500 mg. In some embodiments, a combination therapy as described herein comprises genolimzumab formulated for IV infusion and administration at a dose of 150-500 mg. In some embodiments, a combination therapy as described herein comprises genolimzumab formulated for IV infusion and administration at a dose of 200-300 mg.

In some embodiments, a combination therapy as described herein comprises retifanlimab formulated for IV infusion and administration at a dose of 500 mg. In some embodiments, a combination therapy as described herein comprises retifanlimab formulated for IV administration. In some embodiments, the immune checkpoint inhibitor comprises retifanlimab.

In some embodiments, a combination therapy as described herein comprises retifanlimab formulated for IV infusion and administration at a dose of 250 mg. In some embodiments, a combination therapy as described herein comprises retifanlimab formulated for IV infusion and administration at a dose of 100 mg. In some embodiments, a combination therapy as described herein comprises retifanlimab formulated for IV infusion and administration at a dose of 750 mg. In some embodiments, a combination therapy as described herein comprises retifanlimab formulated for IV infusion and administration at a dose of 1000 mg. In some embodiments, a combination therapy as described herein comprises retifanlimab formulated for IV infusion and administration at a dose of 100-1000 mg. In some embodiments, a combination therapy as described herein comprises retifanlimab formulated for IV infusion and administration at a dose of 250-500 mg.

In some embodiments, a combination therapy as described herein comprises avelumab formulated for IV infusion and administration at a dose of 800 mg. In some embodiments, a combination therapy as described herein comprises avelumab formulated for IV administration. In some embodiments, the immune checkpoint inhibitor comprises avelumab.

In some embodiments, a combination therapy as described herein comprises avelumab formulated for IV infusion and administration at a dose of 400 mg. In some embodiments, a combination therapy as described herein comprises avelumab formulated for IV infusion and administration at a dose of 200 mg. In some embodiments, a combination therapy as described herein comprises avelumab formulated for IV infusion and administration at a dose of 1200 mg. In some embodiments, a combination therapy as described herein comprises avelumab formulated for IV infusion and administration at a dose of 1600 mg. In some embodiments, a combination therapy as described herein comprises avelumab formulated for IV infusion and administration at a dose of 200-1600 mg. In some embodiments, a combination therapy as described herein comprises avelumab formulated for IV infusion and administration at a dose of 400-800 mg.

In some embodiments, a combination therapy as described herein comprises atezolizumab formulated for IV infusion and administration at a dose of 840 mg. In some embodiments, a combination therapy as described herein comprises atezolizumab formulated for IV infusion and administration at a dose of 1200 mg. In some embodiments, a combination therapy as described herein comprises atezolizumab formulated for IV infusion and administration at a dose of 1580 mg. In some embodiments, a combination therapy as described herein comprises atezolizumab formulated for IV administration. In some embodiments, the immune checkpoint inhibitor comprises atezolizumab.

In some embodiments, a combination therapy as described herein comprises atezolizumab formulated for IV infusion and administration at a dose of 400 mg. In some embodiments, a combination therapy as described herein comprises atezolizumab formulated for IV infusion and administration at a dose of 1000 mg. In some embodiments, a combination therapy as described herein comprises atezolizumab formulated for IV infusion and administration at a dose of 1400 mg. In some embodiments, a combination therapy as described herein comprises atezolizumab formulated for IV infusion and administration at a dose of 1800 mg. In some embodiments, a combination therapy as described herein comprises atezolizumab formulated for IV infusion and administration at a dose of 400-1400 mg. In some embodiments, a combination therapy as described herein comprises atezolizumab formulated for IV infusion and administration at a dose of 840-1680 mg.

In some embodiments, a combination therapy as described herein comprises durvalumab formulated for IV infusion and administration at a dose of 1500 mg. In some embodiments, a combination therapy as described herein comprises durvalumab formulated for IV infusion and administration at a dose of 1,120 mg. In some embodiments, a combination therapy as described herein comprises durvalumab formulated for IV infusion and administration at a dose of 20 mg/kg weight of the subject. In some embodiments, a combination therapy as described herein comprises durvalumab formulated for IV infusion and administration at a dose of 10 mg/kg weight of the subject. In some embodiments, a combination therapy as described herein comprises durvalumab formulated for IV administration. In some embodiments, the immune checkpoint inhibitor comprises durvalumab.

In some embodiments, a combination therapy as described herein comprises durvalumab formulated for IV infusion and administration at a dose of 1000 mg. In some embodiments, a combination therapy as described herein comprises durvalumab formulated for IV infusion and administration at a dose of 1,300 mg. In some embodiments, a combination therapy as described herein comprises durvalumab formulated for IV infusion and administration at a dose of 1750 mg. In some embodiments, a combination therapy as described herein comprises durvalumab formulated for IV infusion and administration at a dose of 2000 mg. In some embodiments, a combination therapy as described herein comprises durvalumab formulated for IV infusion and administration at a dose of 1000-2000 mg. In some embodiments, a combination therapy as described herein comprises durvalumab formulated for IV infusion and administration at a dose of 10 mg/kg-20 mg/kg weight of the subject. In some embodiments, a combination therapy as described herein comprises durvalumab formulated for IV infusion and administration at a dose of 1,120-1,500 mg.

In some embodiments, a combination therapy as described herein comprises sugemalimab formulated for IV infusion and administration at a dose of 1200 mg. In some embodiments, a combination therapy as described herein comprises sugemalimab formulated for IV administration. In some embodiments, the immune checkpoint inhibitor comprises sugemalimab.

In some embodiments, a combination therapy as described herein comprises sugemalimab formulated for IV infusion and administration at a dose of 600 mg. In some embodiments, a combination therapy as described herein comprises sugemalimab formulated for IV infusion and administration at a dose of 300 mg. In some embodiments, a combination therapy as described herein comprises sugemalimab formulated for IV infusion and administration at a dose of 1500 mg. In some embodiments, a combination therapy as described herein comprises sugemalimab formulated for IV infusion and administration at a dose of 1750 mg. In some embodiments, a combination therapy as described herein comprises sugemalimab formulated for IV infusion and administration at a dose of 300 mg-1750 mg. In some embodiments, a combination therapy as described herein comprises sugemalimab formulated for IV infusion and administration at a dose of 1,200-1,500 mg.

In some embodiments, a combination therapy as described herein comprises envafolimab formulated for IV infusion and administration at a dose of 400 mg. In some embodiments, a combination therapy as described herein comprises envafolimab formulated for IV administration. In some embodiments, the immune checkpoint inhibitor comprises envafolimab.

In some embodiments, a combination therapy as described herein comprises envafolimab formulated for IV infusion and administration at a dose of 200 mg. In some embodiments, a combination therapy as described herein comprises envafolimab formulated for IV infusion and administration at a dose of 100 mg. In some embodiments, a combination therapy as described herein comprises envafolimab formulated for IV infusion and administration at a dose of 600 mg. In some embodiments, a combination therapy as described herein comprises envafolimab formulated for IV infusion and administration at a dose of 800 mg. In some embodiments, a combination therapy as described herein comprises envafolimab formulated for IV infusion and administration at a dose of 100-800 mg. In some embodiments, a combination therapy as described herein comprises envafolimab formulated for IV infusion and administration at a dose of 400-600 mg.

In some embodiments, a combination therapy as described herein comprises cosibelimab formulated for IV infusion and administration at a dose of 1200 mg. In some embodiments, a combination therapy as described herein comprises cosibelimab formulated for IV administration. In some embodiments, the immune checkpoint inhibitor comprises cosibelimab.

In some embodiments, a combination therapy as described herein comprises cosibelimab formulated for IV infusion and administration at a dose of 600 mg. In some embodiments, a combination therapy as described herein comprises cosibelimab formulated for IV infusion and administration at a dose of 300 mg. In some embodiments, a combination therapy as described herein comprises cosibelimab formulated for IV infusion and administration at a dose of 1500 mg. In some embodiments, a combination therapy as described herein comprises cosibelimab formulated for IV infusion and administration at a dose of 1,800 mg. In some embodiments, a combination therapy as described herein comprises cosibelimab formulated for IV infusion and administration at a dose of 300-1,800 mg. In some embodiments, a combination therapy as described herein comprises cosibelimab formulated for IV infusion and administration at a dose of 1,200-1,500 mg.

In some embodiments, a combination therapy as described herein comprises an anti-IL-2 antibody comprising a heavy chain variable region (VH) comprising heavy chain complementarity determining regions (HCDRs) HCDR1, HCDR2 and HCDR3 wherein said HCDR1 comprises the amino acid sequence of SEQ ID NO:62, said HCDR2 comprises the amino acid sequence of SEQ ID NO:63, said HCDR3 comprises the amino acid sequence of SEQ ID NO:64. In some embodiments, a combination therapy as described herein comprises an anti-IL-2 antibody comprising a VH amino acid sequence as set forth in SEQ ID NO: 26. In some embodiments, a combination therapy as described herein comprises an anti-IL-2 antibody comprising a full length heavy chain having an amino acid sequence as set forth in SEQ ID NO:72.

In some embodiments, a combination therapy as described herein comprises an anti-IL-2 antibody comprising a light chain variable region (VL) comprising light chain complementarity determining regions (LCDRs) LCDR1, LCDR2 and LCDR3, said LCDR1 comprises the amino acid sequence of SEQ ID NO:65, said LCDR2 comprises the amino acid sequence of DAS, and said LCDR3 comprises the amino acid sequence of SEQ ID NO: 67. In some embodiments, a combination therapy as described herein comprises an anti-IL-2 antibody comprising a VL amino acid sequence as set forth in SEQ ID NO: 27. In some embodiments, a combination therapy as described herein comprises an anti-IL-2 antibody comprising a full length light chain having an amino acid sequence as set forth in SEQ ID NO:73.

In some embodiments, a combination therapy as described herein comprises an anti-IL-2 antibody comprising a heavy chain variable region (VH) comprising heavy chain complementarity determining regions (HCDRs) HCDR1, HCDR2 and HCDR3 wherein said HCDR1 comprises the amino acid sequence of SEQ ID NO:62, said HCDR2 comprises the amino acid sequence of SEQ ID NO:63, said HCDR3 comprises the amino acid sequence of SEQ ID NO:64, and a light chain variable region (VL) comprising light chain complementarity determining regions (LCDRs) LCDR1, LCDR2 and LCDR3, said LCDR1 comprises the amino acid sequence of SEQ ID NO:65, said LCDR2 comprises the amino acid sequence of DAS, and said LCDR3 comprises the amino acid sequence of SEQ ID NO: 67. In some embodiments, a combination therapy as described herein comprises an anti-IL-2 antibody comprising a VH amino acid sequence as set forth in SEQ ID NO: 26 and a VL amino acid sequence as set forth in SEQ ID NO: 27. In some embodiments, a combination therapy as described herein comprises an anti-IL-2 antibody comprising a full length heavy chain having an amino acid sequence as set forth in SEQ ID NO:72 and a full length light chain having an amino acid sequence as set forth in SEQ ID NO:73.

In some embodiments, a combination therapy as described herein comprises an anti-IL-2 antibody comprising an IgG, IgA, IgM, IgE, IgD, a Fv, a scFv, a Fab, a F(ab′) 2, a minibody, a diabody, or a triabody. In some embodiments, a combination therapy as described herein comprises an anti-IL-2 antibody comprising a heavy chain comprising a mutation that reduces binding to an Fcγ receptor. In some embodiments, the mutation comprises a L234A mutation. In other embodiments, the mutation comprises a L235A mutation. In other embodiments, the mutation comprises both L234A and L235A mutations. In some embodiments, a combination therapy as described herein comprises an anti-IL-2 antibody that is an human IgG1 antibody with a LALA Fc mutation.

In some embodiments, a combination therapy comprises use of an anti-IL-2 antibody or composition thereof as described herein, and at least two checkpoint inhibitors or a composition thereof. In some embodiments, a combination therapy comprises use of an anti-IL-2 antibody or composition thereof and IL-2 as described herein, and at least two checkpoint inhibitors or a composition thereof.

In some embodiments, a combination therapy comprises a second composition comprising one or more checkpoint inhibitors, as described herein.

In some embodiments, a combination therapy comprises use of anti-IL-2 antibody BDG17.069 or composition thereof; and IL-2 as described herein; and a checkpoint inhibitor or a composition thereof as described herein. In some embodiments, a combination therapy comprises use of BDG17.069 or composition thereof; and a low dose of IL-2 as described herein; and a checkpoint inhibitor or a composition thereof as described herein. In some embodiments, a combination therapy comprises use of BDG17.069 or composition thereof; and a low dose of IL-2 as described herein; and a checkpoint inhibitor or a composition thereof, wherein said checkpoint inhibitor is selected from a PD-1 inhibitor, a PD-L1, a CTLA-4 inhibitor, a TIGIT inhibitor, a TIM-3 inhibitor, a B7-H3 inhibitor, a CD73 inhibitor, a LAG3 inhibitor, a CD27 inhibitor, a CD70 inhibitor, a 4-1BB inhibitor, a GITR inhibitor, a OX40 inhibitor, a SIRP-alpha (CD47) inhibitor, a CD39 inhibitor, a ILDR2 inhibitor, a VISTA inhibitor, a BTLA inhibitor, and a VTCN-1 inhibitor.

In some embodiments, a combination therapy comprises use of BDG17.069 or composition thereof; and a low dose of IL-2 as described herein; and a checkpoint inhibitor or a composition thereof, wherein said checkpoint comprises PD-L1. In some embodiments, a combination therapy comprises use of BDG17.069 or composition thereof; and a low dose of IL-2 (aldesleukin); and avelumab or a composition thereof. In certain embodiments of a combination therapy comprising BDG17.069 or composition thereof; and a low dose of IL-2 (aldesleukin); and avelumab or a composition thereof, the IL-2 is administered by subcutaneous injection. In some embodiments, the low dose of IL-2 is a loading dose. In other embodiments, the low dose of IL-2 is a booster dose.

In certain embodiments, the terms “IL-2” and “aldesleukin” may be used interchangeably having all the same meanings and qualities.

Thus, in some embodiments, a combination therapy comprises the use of BDG17.069 or composition thereof; and a loading dose of IL-2 as described herein; and a checkpoint inhibitor or a composition thereof, wherein said checkpoint comprises PD-L1. In some embodiments, a combination therapy comprises use of BDG17.069 or composition thereof; and a loading dose of IL-2 (aldesleukin); and avelumab or a composition thereof. In certain embodiments of a combination therapy comprising BDG17.069 or composition thereof; and a loading dose of IL-2 (aldesleukin); and avelumab or a composition thereof, the IL-2 is administered by subcutaneous injection. In some embodiments, the dose of IL-2 is a loading dose. In other embodiments, the dose of IL-2 is a booster dose.

In some embodiments, a combination therapy comprises use of BDG17.069 or composition thereof; and a booster dose of IL-2 as described herein; and a checkpoint inhibitor or a composition thereof, wherein said checkpoint comprises PD-L1. In some embodiments, a combination therapy comprises use of BDG17.069 or composition thereof; and a booster dose of IL-2 (aldesleukin); and avelumab or a composition thereof. In certain embodiments of a combination therapy comprising BDG17.069 or composition thereof; and a booster dose of IL-2 (aldesleukin); and avelumab or a composition thereof, the IL-2 is administered by subcutaneous injection.

In some embodiments of a combination therapy, the IL-2 administered comprises a low dose of IL-2. In some embodiments of a combination therapy, the IL-2 administered is by subcutaneous administration. In some embodiments of a combination therapy, the IL-2 administered comprises a low dose of IL-2 administered by subcutaneous administration. In some embodiments of a combination therapy, an anti-IL-2 antibody and IL-2 are comprised in the same composition as a checkpoint inhibitor. In some embodiments, an anti-IL-2 antibody and IL-2 are comprised in different compositions from each other and from a checkpoint inhibitor. In some embodiments, an anti-IL-2 antibody, IL-2, and a checkpoint inhibitor are comprised in the same composition. In some embodiments, an anti-IL-2 antibody and IL-2 are comprised in a composition, and a checkpoint inhibitor is comprised in a different composition. In some embodiments, an anti-IL-2 antibody and a checkpoint inhibitor are comprised in a composition, and IL-2 is comprised in a different composition.

In some embodiments of a combination therapy, BDG17.069 and aldesleukin are comprised in the same composition as a PD-L1 checkpoint inhibitor. In some embodiments of a combination therapy, BDG17.069 and aldesleukin are comprised in the same composition as avelumab. In some embodiments, BDG17.069 and aldesleukin are comprised in different compositions from each other and from avelumab. In some embodiments, BDG17.069 and aldesleukin and avelumab are comprised in the same composition. In some embodiments, BDG17.069 and aldesleukin are comprised in a composition, and avelumab is comprised in a different composition. In some embodiments, BDG17.069 and avelumab are comprised in a composition, and aldesleukin is comprised in a different composition. In some embodiments of a combination therapy, the order of administration of an anti-IL-2 antibody or a composition thereof and a checkpoint inhibitor or a composition thereof, may be in any order. In some embodiments of a combination therapy, the order of administration of BDG17.069 or a composition thereof or a composition thereof and avelumab or a composition thereof, may be in any order. In some embodiments of a combination therapy, the order of administration of an anti-IL-2 antibody or a composition thereof, IL-2 or a composition thereof, and a checkpoint inhibitor or a composition thereof, may be in any order. In some embodiments of a combination therapy, the order of administration of BDG17.069 or a composition thereof, aldesleukin or a composition thereof, and avelumab or a composition thereof, may be in any order. For example, but not limited to the anti-IL-2 antibody may be administered prior to, concurrent with, or following administration of the checkpoint inhibitor. Similarly, a combination of an anti-IL-2 antibody and IL-2 may be administered prior to, concurrent with, or following administration of the checkpoint inhibitor. For example, but not limited to the BDG17.069 may be administered prior to, concurrent with, or following administration of the avelumab. Similarly, a combination of BDG17.069 and aldesleukin may be administered prior to, concurrent with, or following administration of the avelumab. In some embodiments, the anti-IL-2 antibody may be administered prior to, concurrent with, or following administration of the at least two checkpoint inhibitors. Similarly, a combination of an anti-IL-2 antibody and IL-2 may be administered prior to, concurrent with, or following administration of the at least two checkpoint inhibitors.

In some embodiments, administration of a combination therapy with a checkpoint inhibitor comprises concurrent administration of an anti-IL-2 antibody or a composition thereof and the checkpoint inhibitor. In some embodiments, administration of a combination therapy with a checkpoint inhibitor comprises concurrent administration of BDG17.069 or a composition thereof and avelumab. In some embodiments, administration of a combination therapy with a checkpoint inhibitor comprises concurrent administration of an anti-IL-2 antibody and IL-2, or composition(s) thereof and the checkpoint inhibitor. In some embodiments, administration of a combination therapy with a checkpoint inhibitor comprises concurrent administration of BDG17.069 and aldesleukin, or composition(s) thereof and avelumab. In some embodiments, administration of a combination therapy with a checkpoint inhibitor comprises prior administration of an anti-IL-2 antibody or a composition thereof before the checkpoint inhibitor. In some embodiments, administration of a combination therapy with a checkpoint inhibitor comprises prior administration of BDG17.069 or a composition thereof before the avelumab. In some embodiments, administration of a combination therapy with a checkpoint inhibitor comprises prior administration of an anti-IL-2 antibody and IL-2, or composition(s) thereof before the checkpoint inhibitor. In some embodiments, administration of a combination therapy with a checkpoint inhibitor comprises prior administration of BDG17.069 and aldesleukin, or composition(s) thereof before the avelumab. In some embodiments, administration of a combination therapy with a checkpoint inhibitor comprises later administration of an anti-IL-2 antibody or a composition thereof following administration of the checkpoint inhibitor. In some embodiments, administration of a combination therapy with a checkpoint inhibitor comprises later administration of BDG17.069 or a composition thereof following administration of the avelumab. In some embodiments, administration of a combination therapy with a checkpoint inhibitor comprises later administration of an anti-IL-2 antibody and IL-2, or composition(s) thereof following the administration of the checkpoint inhibitor. In some embodiments, administration of a combination therapy with a checkpoint inhibitor comprises later administration of BDG17.069 and aldesleukin, or composition(s) thereof following the administration of the avelumab.

In some embodiments, a combination therapy comprises use of a checkpoint inhibitor and an anti-IL-2 antibody comprising a heavy chain variable region having the sequence of SEQ ID NO:26. In some embodiments, a combination therapy comprises use of a checkpoint inhibitor and an anti-IL-2 antibody comprising a light chain variable region having the sequence of SEQ ID NO:27. In some embodiments, a combination therapy comprises use of a checkpoint inhibitor and an anti-IL-2 antibody comprising a heavy chain variable region and a light chain variable region having the sequences of SEQ ID NOs: 26 and 27. In some embodiments, a combination therapy comprises use of a checkpoint inhibitor and an anti-IL-2 antibody comprising a heavy chain variable domain comprising CDR1, CDR2 and CDR3 regions comprising amino acid sequences of SEQ ID NOs: 62-64 respectively. In some embodiments, a combination therapy comprises use of a checkpoint inhibitor and an anti-IL-2 antibody comprising a light chain variable domain comprising CDR1, CDR2 and CDR3 regions comprising amino acid sequences of SEQ ID NO: 65, DAS, and SEQ ID NO: 67, respectively. In some embodiments, a combination therapy comprises use of a checkpoint inhibitor and an anti-IL-2 antibody comprising a heavy chain variable domain comprising CDR1, CDR2 and CDR3 regions comprising amino acid sequences of SEQ ID NOs: 62-64, respectively, and a light chain variable domain comprising CDR1, CDR2, and CDR3 regions comprising amino acid sequences of SEQ ID NO: 65, DAS, and SEQ ID NO: 67, respectively.

In some embodiments, a combination therapy comprises use of a checkpoint inhibitor and an anti-IL-2 antibody comprising anti-IL-2 clone BDG 17.069.

In some embodiments, a combination therapy comprises use of a checkpoint inhibitor; and an anti-IL-2 antibody comprising clone BDG 17.069; and IL-2.

In certain embodiments, use of a combination therapy is for treating an unresectable locally advanced or metastatic NSCLC. In certain embodiments, use of a combination therapy is for treating an unresectable locally advanced or metastatic NSCLC that progressed after prior checkpoint inhibitor therapy. In certain embodiments, use of a combination therapy is for treating an unresectable locally advanced or metastatic NSCLC, wherein the NSCLC is PD-L1 positive NSCLC. In certain embodiments, use of a combination therapy is for treating an unresectable locally advanced or metastatic NSCLC, wherein the NSCLC is PD-1 positive NSCLC. In some embodiments, the NSCLC comprises a squamous NSCLC. In other embodiments, the NSCLC comprises a non-squamous NSCLC.

### Engineered Anti-IL-2 Antibodies

The present disclosure provides engineered anti-human IL-2 antibodies that bind human IL-2 with high affinity (e.g., 12.7 μM to 48 μM) to a pre-defined binding epitope. The antibodies bind to IL-2 in a manner that completely prevents CD25 binding, yet spares the binding of IL-2 to CD122, thereby modulating immune responses towards immune stimulation by directly activating and expanding effector cells without interacting with CD25-expressing cells (e.g., regulatory T-cells, short lived cytotoxic T-cells, pulmonary endothelial cells and vascular endothelial cells). Thus, the antibody/IL-2 complex would drive a robust immune response to clear a tumor by expanding and activating effector cells such as NK cells, central memory T cells and tumor-specific T-cells while inhibiting IL-2 activation induced cell death of the short lived CD25+ cytotoxic T-cells that are important for tumor clearance. The antibody/IL-2 complex would also decrease immunosuppression caused by the regulatory arm of the immune system. Moreover, the antibody/IL-2 complex prevent undesired interactions of IL-2 with vascular and pulmonary CD25-expressing cells, thereby preventing severe syndromes of IL-2 induced vascular leakage and IL-2 induced pulmonary edema. In some embodiments, the activity of the engineered anti-IL-2 antibodies described herein is dependent on the pre-defined epitope to which they are designed to bind.

In some embodiments, the IL-2 antibodies disclosed herein, block IL-2 binding to CD25. In some embodiments, the IL-2 antibodies disclosed herein binds IL-2 and prevent newly secreted endogenous IL-2 from binding to Tregs, effectively blocking the negative feedback loop of IL-2 to Tregs. In some embodiments, the IL-2 antibodies disclosed herein prevent Treg expansion. In some embodiments, the IL-2 antibodies disclosed herein block IL-2 binding to vascular endothelium. In some embodiments, the IL-2 antibodies disclosed herein block IL-2 binding to pulmonary endothelium. In some embodiments, the IL-2 antibodies disclosed herein block IL-2 binding to vascular and pulmonary endothelium.

In some embodiments, an anti-human anti-IL-2 antibody described herein inhibits binding of IL-2 with an IL-2 receptor alpha (IL-2 Ra, i.e., CD25) subunit and therefore inhibits binding to the trimer IL-2 Rαβγ receptor. In certain embodiments, anti-IL-2 antibodies that inhibit binding of IL-2 with a trimer IL-2 receptor (IL-2 Rαβγ) do not inhibit binding of IL-2 with the dimer IL-2 receptor (IL-2 Rβγ).

Targeting IL-2 to different cell populations can be used to either modulate the immune response toward immunosuppression or towards immune activation. The anti-human IL-2 antibodies disclosed herein are designed to bind with high affinity to an IL-2 epitope that blocks IL-2 binding to CD25. As a result, IL-2 is prevented from binding to short-lived CD8+ cytotoxic T cells or regulatory T cells that express high level of CD25 but is redirected to preferentially bind to effector T cells to stimulate enhanced immune response. Moreover, since IL-2 binding to CD25-expressing endothelial cells is also blocked, IL-2 induced pulmonary edema and vascular leaking would also be prevented.

In some embodiments, the present disclosure provides an anti-IL-2 antibody designed to enhance T cell immune response and to prevent severe edema symptoms of acute pneumonia induced by IL-2. The anti-IL-2 antibody binds specifically to human IL-2 with high affinity at a pre-defined epitope that blocks IL-2 binding to the alpha chain of the IL-2 receptors (CD25) while sparing binding to the main signaling beta chain and gamma chain complex of the receptor (CD122/CD132). Consequently, in the presence of such antibody, IL-2 would be directed to immune cells responsible for tumor clearance and away from cells that slow the immune response or cause the edema. The formation of this IL-2/antibody immunocomplex will direct IL-2 to bind and activate exclusively naive and memory T lymphocytes, NK cells, and Natural Killer T lymphocytes while preventing activation of regulatory T cells and apoptosis of short-lived CD25+ cytotoxic T effector cells. Altogether, the end result is an effective immune response, for example, tumor clearance. In addition, this treatment will prevent the toxicity caused by IL-2 binding to endothelial CD25 expressing cells. In other embodiments, treatment with the anti-IL-2 antibodies disclosed herein would be effective in preventing the toxicity caused by IL-2 binding to endothelial CD25 expressing cells, e.g., pulmonary edema, or IL-2-induced vascular leakage.

The cytokine IL-2 is critical for the expansion of T cells. However, in addition to its pro-stimulatory role IL-2 also induces some adverse side effects like lung edema and vascular leak syndrome through its binding to endothelium expressing the CD25 receptor.

In some embodiments, the present disclosure provides engineered anti-IL-2 antibodies resulting from introducing amino acid variations to a parent anti-IL-2 antibody. In some embodiments, the one or more of the amino acid variations are introduced in a CDR region. In other embodiments, the one or more amino acid variations are introduced within a framework (FR) region. In yet another embodiment, the amino acid variations are introduced to both the CDR region and the framework (FR) region. One of ordinary skill in the art would readily employ various standard techniques known in the art to introduce amino acid variations into an anti-IL-2 antibody and then test the resulting modified antibodies for any changes of binding to IL-2. While standard techniques may be used, the resultant binding pattern of the newly created antibodies is not predictable and must be analyzed to determine functionality.

In certain embodiments, the present disclosure provides polypeptides comprising the VH and VL domains which could be dimerized under suitable conditions. For example, the VH and VL domains may be combined in a suitable buffer and dimerized through appropriate interactions such as hydrophobic interactions. In other embodiments, the VH and VL domains may be combined in a suitable buffer containing an enzyme and/or a cofactor which can promote dimerization of the VH and VL domains. In other embodiments, the VH and VL domains may be combined in a suitable vehicle that allows them to react with each other in the presence of a suitable reagent and/or catalyst.

In certain embodiments, the VH and VL domains may be contained within longer polypeptide sequences, which may include for example but not limited to, constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g., CH1, CH2, CH3, CH4, Ck, Cl). In some embodiments, the longer polypeptides may comprise multiple copies of one or both of the VH and VL domains generated according to the method disclosed herein; for example, when the polypeptides generated herein are used to form a diabody or a triabody.

In some embodiments, the Fc region comprises at least one mutation that reduces Fc-gamma binding, i.e., binding to a Fcγ receptor (FcγRs). In some embodiments, reduced binding is abolished binding, which binding to the Fcγ receptor is not detectable. In some embodiments, reduced binding reduces the binding affinity to a Fcγ receptor. In some embodiments, reduced binding reduces the on rate for binding to a Fcγ receptor. In some embodiments, reduced binding reduces the off rate of binding to a Fcγ receptor. In some embodiments, a mutation that reduces the Fc-gamma binding comprises L234A, L235A mutations, also known as LALA mutations. In some embodiments, a mutation that reduces the Fc-gamma binding comprises a P329G mutation in addition to the L234A, L235A mutations. In some embodiments, an antibody described herein comprises a heavy chain comprising a mutation that reduces binding to Fcγ receptor.

In some embodiments, the present disclosure provides an engineered (or modified) anti-IL-2 antibody, wherein the antibody comprises a heavy chain variable region having the sequence of SEQ ID NO: 26. In some embodiments, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In other embodiments, the engineered antibody can be part of a minibody, a diabody, or a triabody antibody.

In some embodiments, the present disclosure provides an engineered (or modified) anti-IL-2 antibody, wherein the antibody comprises a light chain variable region having the sequence of SEQ ID NO: 27. In some embodiments, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In other embodiments, the engineered antibody can be part of a minibody, a diabody, or a triabody antibody.

In some embodiments, the present disclosure provides an engineered (or modified) anti-IL-2 antibody, wherein the antibody comprises a heavy chain variable region and a light chain variable region having the sequences of on SEQ ID NOs: 26 and 27. In some embodiments, the engineered anti-IL-2 antibody comprises the sequences of SEQ ID NOs: 26 and 27.

In some embodiments, an isolated anti-IL-2 antibody comprising a heavy chain variable region (VH) and a light chain variable region (VL), wherein said VH comprises heavy chain complementarity determining regions (HCDRs) HCDR1, HCDR2 and HCDR3, said VL comprises light chain complementarity determining regions (LCDRs) LCDR1, LCDR2 and LCDR3, wherein the HCDR1 comprises the amino acid sequence of SEQ ID NO: 62, the HCDR2 comprises the amino acid sequence of SEQ ID NO:63, the HCDR3 comprises the amino acid sequence of SEQ ID NO:64, the LCDR1 comprises the amino acid sequence of SEQ ID NO:65, the LCDR2 comprises the amino acid sequence of DAS, the LCDR3 comprises the amino acid sequence of SEQ ID NO:67.

In some embodiments, the VH and VL have the amino acid sequences wherein the VH comprises the amino acid sequence of SEQ ID NO:26, the VL comprises the amino acid sequence of SEQ ID NO:27.

In some embodiments, an antibody comprises a heavy chain sequence and a light chain sequence, said heavy chain sequence set forth in SEQ ID NO: 72 and said light chain sequence set forth in SEQ ID NO: 73. In some embodiments, an antibody comprising a heavy chain sequence and a light chain sequence, said heavy chain sequence set forth in SEQ ID NO: 72 and said light chain sequence set forth in SEQ ID NO: 73.

In some embodiments, the engineered antibody can be an IgG, IgA, IgM, IgE, IgD, a Fv, a scFv, a Fab, or a F(ab′)₂. The IgG can be of the subclass of IgG1, IgG2, IgG3, or IgG4. In other embodiments, the engineered antibody can be part of a minibody, a diabody, or a triabody antibody.

In some embodiments, the present disclosure also provides isolated polynucleotide sequence encoding a heavy chain variable region of an anti-IL-2 antibody, wherein the heavy chain variable region comprises the amino acid sequence of SEQ ID NO: 26. In other embodiments, the present disclosure also provides a vector comprising the above-mentioned polynucleotide sequences. In view of the amino acid sequences disclosed herein, one of ordinary skill in the art would readily construct a vector or plasmid to encode for the amino acid sequences. In other embodiments, the present disclosure also provides a host cell comprising the vector provided herein. Depending on the uses and experimental conditions, one of skill in the art would readily employ a suitable host cell to carry and/or express the above-mentioned polynucleotide sequences.

In some embodiments, the present disclosure also provides isolated polynucleotide sequence encoding a light chain variable region of an anti-IL-2 antibody, wherein the light chain variable region comprises the amino acid sequence of SEQ ID NO: 27. In other embodiments, the present disclosure also provides a vector comprising the above-mentioned polynucleotide sequences. In view of the amino acid sequences disclosed herein, one of ordinary skill in the art would readily construct a vector or plasmid to encode for the amino acid sequences. In other embodiments, the present disclosure also provides a host cell comprising the vector provided herein. Depending on the uses and experimental conditions, one of skill in the art would readily employ a suitable host cell to carry and/or express the above-mentioned polynucleotide sequences.

In view of the sequences for the heavy chain variable regions and light chain variable regions disclosed herein, one of ordinary skill in the art would readily employ standard techniques known in the art to construct an anti-IL-2 scFv. In some embodiments, polynucleotide sequences encoding for such anti-IL-2 scFv could have the sequence of SEQ ID NO: 35.

In certain embodiments, an isolated polynucleotide sequence disclosed herein, encoding the heavy chain variable region of an anti-IL-2 antibody, comprises a VH amino acid sequence set forth in the amino acid sequence of SEQ ID NO: 26. In some embodiments, a vector comprises the polynucleotide sequence of SEQ ID NO: 26. In some embodiments, a host cell comprising the vector comprising the polynucleotide sequence of SEQ ID NO: 26.

In certain embodiments, an isolated polynucleotide sequence disclosed herein, encoding the light chain variable region of an anti-IL-2 antibody, comprises a VL amino acid sequence as set forth in the amino acid sequence of SEQ ID NO: 27. In some embodiments, a vector comprises the polynucleotide sequence comprising the amino acid sequence of SEQ ID NO: 27. In some embodiments, a host cell comprises a vector comprising the polynucleotide sequence encoding the amino acid sequence of SEQ ID NO: 27. In some embodiments, an isolated polynucleotide sequence encodes an anti-IL-2 scFv, wherein the polynucleotide sequence encodes SEQ ID NO: 35. In some embodiments, a vector comprises an isolated polynucleotide sequence encodes an anti-IL-2 scFv, wherein the polynucleotide sequence encodes SEQ ID NO: 35. In some embodiments, a host cell comprises a vector comprising an isolated polynucleotide sequence encoding an anti-IL-2 scFv, wherein the polynucleotide sequence encodes SEQ ID NO: 35.

In other embodiments, the present disclosure also provides an isolated anti-IL-2 antibody, wherein the antibody comprises a heavy chain variable region having complementarity determining region (CDR) 1, CDR2 and CDR3. In some embodiments, the CDR1, CDR2 and CDR3 comprise the amino acid sequences of SEQ ID NOs: 62-64 respectively. In some embodiments, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In some embodiments, the present disclosure also encompasses a composition comprising the above-mentioned antibody and a pharmaceutically acceptable carrier.

In other embodiments, the present disclosure also provides an isolated anti-IL-2 antibody, wherein the antibody comprises a light chain variable region having complementarity determining region (CDR) 1, CDR2 and CDR3. In some embodiments, the CDR1, CDR2 and CDR3 comprise amino acid sequences of SEQ ID NO: 65, DAS, and SEQ ID NO: 67 respectively. In some embodiments, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In some embodiments, the present disclosure also encompasses a composition comprising the above-mentioned antibody and a pharmaceutically acceptable carrier.

In other embodiments, the present disclosure also provides an isolated anti-IL-2 antibody, wherein the antibody comprises a heavy chain variable region having complementarity determining region (CDR) 1, CDR2 and CDR3, and a light chain variable region having CDR1, CDR2 and CDR3. In some embodiments, the heavy chain CDR1, CDR2 and CDR3 comprise the amino acid sequences of SEQ ID NOs: 62-64, respectively. In some embodiments, the light chain CDR1, CDR2 and CDR3 comprise the amino acid sequences of SEQ ID NO: 65, DAS, and SEQ ID NO: 67, respectively. In some embodiments, the antibody can be an IgG, IgA, IgM, IgE, IgD, a Fv, a scFv, a Fab, a F(ab′)₂, a minibody, a diabody, or a triabody antibody. The IgG can be IgG1, IgG2, IgG3, or an IgG4. In some embodiments, the present disclosure also encompasses a composition comprising the above-mentioned antibody and a pharmaceutically acceptable carrier.

In some embodiments, an anti-IL-2 antibody as described herein is referred to as “BDG17.069” or “AU-007”, which may be used interchangeably with the term “anti-IL-2 antibody”, having all the same qualities and meanings. In addition, the term “imneskibart” may be used interchangeably with the terms “BDG17.069” or “AU-007” having all the same qualities and meanings, wherein imneskibart is the human monoclonal antibody also known as BDG17.069 or AU-007 with drug-like properties that provides advantages over non-natural biologics.

### Pharmaceutical Compositions

In some embodiments, disclosed herein are compositions for therapeutic use. In some embodiments, a composition described herein comprises an anti-IL-2 antibody as disclosed herein and a pharmaceutically acceptable carrier.

As used herein, the terms “composition” and pharmaceutical composition” may in some embodiments, be used interchangeably having all the same qualities and meanings. In some embodiments, disclosed herein is a pharmaceutical composition for the treatment of a condition or disease as described herein.

In some embodiments, disclosed herein are pharmaceutical compositions for use in a combination therapy.

In other embodiments, disclosed herein are compositions for use in treating an unresectable locally advanced or metastatic NSCLC.

In some embodiments, compositions comprise an anti-IL-2 antibody and a pharmaceutically acceptable carrier. In some embodiments, compositions comprise an anti-IL-2 antibody and IL-2, and a pharmaceutically acceptable carrier. In some embodiments, compositions comprise an anti-IL-2 antibody, IL-2, and an immune checkpoint inhibitor, and a pharmaceutically acceptable carrier. In some embodiments, compositions comprise an anti-IL-2 antibody, IL-2, and avelumab, and a pharmaceutically acceptable carrier.

In some embodiments, compositions comprise an anti-IL-2 antibody and a pharmaceutically acceptable carrier. In some embodiments, compositions comprise IL-2, and a pharmaceutically acceptable carrier. In some embodiments, compositions comprise an immune checkpoint inhibitor and a pharmaceutically acceptable carrier. In some embodiments, compositions comprise avelumab and a pharmaceutically acceptable carrier.

In some embodiments, an anti-IL-2 antibody and IL-2 are comprised in the same composition. In some embodiments, an anti-IL-2 antibody and IL-2 are comprised in different compositions. In some embodiments, an anti-IL-2 antibody and an immune checkpoint inhibitor are comprised in the same composition. In some embodiments, an anti-IL-2 antibody and an immune checkpoint inhibitor are comprised in different compositions. In some embodiments, an anti-IL-2 antibody and avelumab are comprised in the same composition. In some embodiments, an anti-IL-2 antibody and avelumab are comprised in different compositions. In some embodiments, an anti-IL-2 antibody, IL-2, and an immune checkpoint inhibitor are comprised in the same composition. In some embodiments, an anti-IL-2 antibody, IL-2 and an immune checkpoint inhibitor are comprised in different compositions. In some embodiments, an anti-IL-2 antibody, IL-2, and avelumab are comprised in the same composition. In some embodiments, an anti-IL-2 antibody, IL-2 and avelumab are comprised in different compositions.

In some embodiments, administration of a combination of an anti-IL-2 antibody and IL-2, or composition(s) thereof are concurrent. In some embodiments, administration of a combination of an anti-IL-2 antibody and IL-2, or composition(s) thereof comprises administration of an anti-IL-2 antibody or a composition thereof, prior to the IL-2 or a composition thereof. In some embodiments, administration of a combination of an anti-IL-2 antibody and IL-2, or composition(s) thereof comprises administration of an anti-IL-2 antibody or a composition thereof, following administration of the IL-2 or a composition thereof.

In some embodiments, administration of a combination of an anti-IL-2 antibody, IL-2, and an immune checkpoint inhibitor or composition(s) thereof are concurrent. In some embodiments, administration of a combination of an anti-IL-2 antibody, IL-2, and an immune checkpoint inhibitor or composition(s) thereof comprises administration of an anti-IL-2 antibody or a composition thereof, prior to the IL-2 and or the immune checkpoint inhibitor, or compositions thereof. In some embodiments, administration of a combination of an anti-IL-2 antibody, IL-2, and an immune checkpoint inhibitor or composition(s) thereof comprises administration of an anti-IL-2 antibody or a composition thereof, following administration of the IL-2 and or an immune checkpoint inhibitor, or a composition thereof.

In some embodiments, administration of a combination of an anti-IL-2 antibody, IL-2, and avelumab or composition(s) thereof are concurrent. In some embodiments, administration of a combination of an anti-IL-2 antibody, IL-2, and avelumab or composition(s) thereof comprises administration of an anti-IL-2 antibody or a composition thereof, prior to the IL-2 and or avelumab, or compositions thereof. In some embodiments, administration of a combination of an anti-IL-2 antibody, IL-2, and avelumab or composition(s) thereof comprises administration of an anti-IL-2 antibody or a composition thereof, following administration of the IL-2 and or avelumab, or a composition thereof.

In some embodiments, administering a loading dose of IL-2 or a composition thereof, comprises administering prior to, concurrent with, or following administration of the anti-IL-2 antibody, avelumab, or both. In certain embodiments of administering an anti-IL-2 antibody, IL-2, and or an immune checkpoint inhibitor, for example avelumab comprises administering a pharmaceutical composition comprising the anti-IL-2 antibody, the IL-2, and or the immune checkpoint inhibitor (e.g., avelumab), wherein the composition further comprises a pharmaceutically acceptable carrier. In some embodiments, each of the anti-IL-2 antibody, the IL-2, and the immune checkpoint inhibitor (e.g., avelumab) are comprised in separate compositions that further comprise a pharmaceutically acceptable carrier.

A skilled artisan would appreciate that a “pharmaceutical composition” may encompass a preparation of one or more of the active ingredients described herein with other chemical components such as physiologically suitable carriers and excipients. The purpose of a pharmaceutical composition is to facilitate administration of an active agent, for example but not limited to an antibody or a compound, to an organism.

In some embodiments, disclosed herein is a pharmaceutical composition for a therapy use treating a subject with a weakened immune system. In some embodiments, disclosed herein is a pharmaceutical composition for a therapy use treating a subject suffering from an unresectable locally advanced or metastatic NSCLC. In some embodiments, disclosed herein is a pharmaceutical composition for use as part of a combination therapy for treating a subject with a weakened immune system. In some embodiments, disclosed herein is a pharmaceutical composition for use as part of a combination therapy for use treating a subject suffering from an unresectable locally advanced or metastatic NSCLC.

A skilled artisan would appreciate that the phrases “physiologically acceptable carrier”, “pharmaceutically acceptable carrier”, “physiologically acceptable excipient”, and “pharmaceutically acceptable excipient”, may be used interchangeably may encompass a carrier, excipient, or a diluent that does not cause significant irritation to an organism and does not abrogate the biological activity and properties of the administered active ingredient.

A skilled artisan would appreciate that an “excipient” may encompass an inert substance added to a pharmaceutical composition to further facilitate administration of an active ingredient. In some embodiments, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences”, Mack Publishing Co., Easton, PA, latest edition, which is incorporated herein by reference.

In some embodiments, the composition as disclosed herein comprises a therapeutic composition. In some embodiments, the composition as disclosed herein comprises a therapeutic efficacy.

### Administration

In some embodiments, methods disclosed herein comprise administering compositions comprising an anti-IL-2 antibody as disclosed herein, in conjunction with a loading dose of IL-2. The method further comprises administering a booster dose of IL-2. In other embodiments, methods disclosed herein administer a combination therapy comprising compositions comprising an anti-IL-2 antibody as disclosed herein and a loading dose of IL-2. The method further comprises administering a booster dose of IL-2.

In some embodiments, methods disclosed herein comprise administering compositions comprising an anti-IL-2 antibody as disclosed herein and an immune checkpoint inhibitor as disclosed herein, in conjunction with a loading dose of IL-2. In some embodiments, the method further comprises administering a booster dose of IL-2. In other embodiments, methods disclosed herein administer a combination therapy comprising compositions comprising an anti-IL-2 antibody as disclosed herein and an immune checkpoint inhibitor as disclosed herein, and a loading dose of IL-2. In some embodiments, the method further comprises administering a booster dose of IL-2.

The anti-IL-2 antibody disclosed herein can be administered to a subject (e.g., a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodology well known in the pharmaceutical art.

The above pharmaceutical compositions comprising the polypeptides disclosed herein can be administered (e.g., to a mammal, a cell, or a tissue) in any suitable manner depending on whether local or systemic treatment is desired. For example, the composition can be administered topically (e.g., ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares. The composition can be delivered by a spraying mechanism or droplet mechanism, or through aerosolization. Delivery can also be directed to any area of the respiratory system (e.g., lungs) via intubation. Alternatively, administration can be intratumoral, e.g., local or intravenous injection.

In certain embodiments, an anti-IL-2 antibody or composition thereof as described herein is administered intravenously (IV). In some embodiments, avelumab or composition thereof is administered intravenously (IV). In some embodiments, an immune checkpoint inhibitor or composition thereof is administered intravenously (IV). In some embodiments, low dose IL-2 or composition thereof is administered subcutaneously. In some embodiments, a loading dose of IL-2 or composition thereof is administered subcutaneously. In some embodiments, a booster dose of IL-2 or composition thereof is administered subcutaneously.

In some embodiments, IL-2 subcutaneous administration is at much lower doses and much less frequently than the approved regimen of intravenously administered aldesleukin. In some embodiments, wherein a subject receives both an anti-IL-2 antibody and IL-2, the route of administration of the anti-IL-2 antibody is by intravenous injection and the route of administration of the IL-2 is by subcutaneous injection.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve preparation of a slow-release or sustained-release system so as to maintain a constant dosage.

In some embodiments, where an anti-IL-2 antibody and IL-2 are administered, they may be administered in the same composition. In some embodiments, where an anti-IL-2 antibody and IL-2 are administered, they may be administered in separate compositions. In some embodiments, where an anti-IL-2 antibody and an immune checkpoint inhibitor, for example avelumab are administered, they may be administered in the same composition. In some embodiments, where an anti-IL-2 antibody and an immune checkpoint inhibitor, for example avelumab are administered, they may be administered in separate compositions.

In some embodiments, IL-2 may be administered prior to, concurrent with, or following the step of administering the anti-IL-2 antibody. In some embodiments, IL-2 administration is prior to administering the anti-IL-2 antibody. In some embodiments, IL-2 administration is concurrent with administering the anti-IL-2 antibody. In some embodiments, IL-2 administration follows the step of administering the anti-IL-2 antibody.

In some embodiments, the immune checkpoint inhibitor or avelumab may be administered prior to, concurrent with, or following the step of administering the anti-IL-2 antibody. In some embodiments, the immune checkpoint inhibitor or avelumab administration is prior to administering the anti-IL-2 antibody. In some embodiments, the immune checkpoint inhibitor or avelumab administration is concurrent with administering the anti-IL-2 antibody. In some embodiments, the immune checkpoint inhibitor or avelumab administration follows the step of administering the anti-IL-2 antibody.

In some embodiments, IL-2 may be administered prior to, concurrent with, or following the step of administering the immune checkpoint inhibitor or avelumab. In some embodiments, IL-2 administration is prior to administering the immune checkpoint inhibitor or avelumab. In some embodiments, IL-2 administration is concurrent with administering the immune checkpoint inhibitor or avelumab. In some embodiments, IL-2 administration follows the step of administering the immune checkpoint inhibitor or avelumab.

In some embodiments, administration of an anti-IL-2 antibody comprises including a loading dose of IL-2 with the anti-IL-2 antibody. In some embodiments, administration of an anti-IL-2 antibody comprises a combination therapy, wherein anti-IL-2 antibody and an IL-2 loading dose is administered to a subject at regular intervals, while an IL-2 booster dose is administered to a subject when there is one or more objective signs of worsening tumor growth kinetics, which in some embodiments comprise (a) previously shrinking tumors becoming stable, (b) an increase in tumor markers, (c) an increase in tumor growth, (d) an increase in appearance of new tumor growth in a tumor that had previously been stable, (e) an increase in appearance of new tumor growth in a tumor that had previously decreased in size, or (f) any combination thereof.

In some embodiments, an anti-IL-2 antibody is administered weekly, bi-weekly (once every two weeks), or once every three weeks. In some embodiments, IL-2 is administered as a one-time loading” dose. In some embodiments, an immune checkpoint inhibitor is administered weekly, bi-weekly, once every three weeks, once every four weeks, once every five weeks, once every six weeks, once every seven weeks, or once every eight weeks. In some embodiments, avelumab is administered weekly, once every 2, 3, 4, 5, 6, 7, or 8 weeks. In some embodiments, the anti-IL-2 antibody, the IL-2, and the immune checkpoint inhibitor, for example avelumab, are administered independent of each other

In some embodiments, therapeutic dosages of anti-IL-2 are administered over a period of months. In some embodiments, therapeutic dosages of anti-IL-2 are administered for up to 3 months. In some embodiments, therapeutic dosages of anti-IL-2 are administered for at least 3 months. In some embodiments, therapeutic dosages of anti-IL-2 are administered for up to 6 months. In some embodiments, therapeutic dosages of anti-IL-2 are administered for at least 6 months. In some embodiments, therapeutic dosages of anti-IL-2 are administered for up to 9 months. In some embodiments, therapeutic dosages of anti-IL-2 are administered for at least 9 months. In some embodiments, therapeutic dosages of anti-IL-2 are administered over a period of up to a year. In some embodiments, therapeutic dosages of anti-IL-2 are administered over a period of at least a year. In some embodiments, therapeutic dosages of anti-IL-2 are administered over a period of up to 2 years. In some embodiments, therapeutic dosages of anti-IL-2 are administered over a period of at least 2 years.

The duration of treatment with immune therapies is not well defined as the treatment duration for these immune therapies is often based on the response or lack of response to the drug/combination therapy. A skilled artisan would appreciate that a reasonable duration of treatment, in some embodiments, comprises about 2 years of therapy for patients who are receiving benefit from the treatment, for example but not limited to where the tumor(s) are stabilized or shrinking. In other embodiments, patients who progress immediately or only with short periods of tumor stabilization or shrinkage have a duration of treatment <2 years, most often prior to receiving 1 year of therapy.

In some embodiments, a combination therapy as described herein has a duration of about one year. In some embodiments, a combination therapy as described herein has a duration of less than one year. In some embodiments, a combination therapy as described herein has a duration of about two years. In some embodiments, a combination therapy as described herein has a duration of less than two years. In some embodiments, a combination therapy as described herein has a duration of about one-two years.

In some embodiments, the duration of administration of booster doses of IL-2 as part of a combination therapy varies between patient to patient. In some embodiments, a patient receives a single booster dose over a 1 year period. In some embodiments, a patient receives a single booster dose over a 2 year period. In some embodiments, a patient receives up to 6 booster doses over a 1 year period. In some embodiments, a patient receives up to 12 booster doses over a 2 year period. In some embodiments, a patient receives 1, 2, 3, 4, 5, or 6 booster doses over a 1 year period. In some embodiments, a patient receives 1, 2, 3, 4, 5, 6, 7, 8, 9, 10, 11, or 12 booster doses over a 2 year period. In some embodiments, a patient receives booster doses intermittently over a 6 month-two year period. In some embodiments, a patient receives booster doses intermittently over a 6-month-2 year period based on a clinician's review of one or more objective signs of worsening tumor growth kinetics.

In some embodiments, therapeutic dosages of avelumab are administered over a period of months. In some embodiments, therapeutic dosages of avelumab are administered for up to 3 months. In some embodiments, therapeutic dosages of avelumab are administered for at least 3 months. In some embodiments, therapeutic dosages of avelumab are administered for up to 6 months. In some embodiments, therapeutic dosages of avelumab are administered for at least 6 months. In some embodiments, therapeutic dosages of avelumab are administered for up to 9 months. In some embodiments, therapeutic dosages of avelumab are administered for at least 9 months. In some embodiments, therapeutic dosages of avelumab are administered over a period of up to a year. In some embodiments, therapeutic dosages of avelumab are administered over a period of at least a year.

In some embodiments, therapeutic dosages of the immune checkpoint inhibitor are administered over a period of months. In some embodiments, therapeutic dosages of the immune checkpoint inhibitor are administered for up to 3 months. In some embodiments, therapeutic dosages of the immune checkpoint inhibitor are administered for at least 3 months. In some embodiments, therapeutic dosages of the immune checkpoint inhibitor are administered for up to 6 months. In some embodiments, therapeutic dosages of the immune checkpoint inhibitor are administered for at least 6 months. In some embodiments, therapeutic dosages of the immune checkpoint inhibitor are administered for up to 9 months. In some embodiments, therapeutic dosages of the immune checkpoint inhibitor are administered for at least 9 months. In some embodiments, therapeutic dosages of the immune checkpoint inhibitor are administered over a period of up to a year. In some embodiments, therapeutic dosages of the immune checkpoint inhibitor are administered over a period of at least a year. In some embodiments, the immune checkpoint inhibitor is a PD-L1 immune checkpoint inhibitor. In other embodiments, the immune checkpoint inhibitor is a PD-1 immune checkpoint inhibitor.

In some embodiments, the dose of an anti-IL-2 antibody disclosed herein is between about 0.5 mg/kg and 12 mg/kg. In some embodiments, the dose of an anti-IL-2 antibody disclosed herein is about 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, and 12 mg/kg.

In some embodiments, the dose of an anti-IL-2 antibody disclosed herein is 0.5 mg/kg. In some embodiments, the dose of an anti-IL-2 antibody disclosed herein is 1.0 mg/kg. In some embodiments, the dose of an anti-IL-2 antibody disclosed herein is 1.5 mg/kg. In some embodiments, the dose of an anti-IL-2 antibody disclosed herein is 2.0 mg/kg. In some embodiments, the dose of an anti-IL-2 antibody disclosed herein is 2.5 mg/kg. In some embodiments, the dose of an anti-IL-2 antibody disclosed herein is 3.0 mg/kg. In some embodiments, the dose of an anti-IL-2 antibody disclosed herein is 3.5 mg/kg. In some embodiments, the dose of an anti-IL-2 antibody disclosed herein is 4.0 mg/kg. In some embodiments, the dose of an anti-IL-2 antibody disclosed herein is 4.5 mg/kg. In some embodiments, the dose of an anti-IL-2 antibody disclosed herein is 5.0 mg/kg. In some embodiments, the dose of an anti-IL-2 antibody disclosed herein is 5.5 mg/kg. In some embodiments, the dose of an anti-IL-2 antibody disclosed herein is 6.0 mg/kg. In some embodiments, the dose of an anti-IL-2 antibody disclosed herein is 6.5 mg/kg. In some embodiments, the dose of an anti-IL-2 antibody disclosed herein is 7.0 mg/kg. In some embodiments, the dose of an anti-IL-2 antibody disclosed herein is 7.5 mg/kg. In some embodiments, the dose of an anti-IL-2 antibody disclosed herein is 8.0 mg/kg. In some embodiments, the dose of an anti-IL-2 antibody disclosed herein is 8.5 mg/kg. In some embodiments, the dose of an anti-IL-2 antibody disclosed herein is 9.0 mg/kg. In some embodiments, the dose of an anti-IL-2 antibody disclosed herein is 9.5 mg/kg. In some embodiments, the dose of an anti-IL-2 antibody disclosed herein is 10.0 mg/kg. In some embodiments, the dose of an anti-IL-2 antibody disclosed herein is 10.5 mg/kg. In some embodiments, the dose of an anti-IL-2 antibody disclosed herein is 11.0 mg/kg. In some embodiments, the dose of an anti-IL-2 antibody disclosed herein is 11.5 mg/kg. In some embodiments, the dose of an anti-IL-2 antibody disclosed herein is 12.0 mg/kg. In some embodiments, the dose of an anti-IL-2 antibody disclosed herein is 12.5 mg/kg.

In some embodiments, the dose of IL-2 comprises a low dose. One skilled in the art would appreciate that a low dose of IL-2 may encompass dose level below those provided in studies currently known in the art. In certain embodiments, an IL-2 dose between about 10×10³ IU/kg-300×10³ IU/kg encompasses a low dose of IL-2. In certain embodiments, an IL-2 dose between about 10×10³ IU/kg-500×10³ IU/kg encompasses a low dose of IL-2. In certain embodiments, an IL-2 dose between about 15×10³ IU/kg-500×10³ IU/kg encompasses a low dose of IL-2. In certain embodiments, an IL-2 dose between about 45×10³ IU/kg-270×10³ IU/kg encompasses a low dose of IL-2. In certain embodiments, an IL-2 dose between about 45×10³ IU/kg-135×10³ IU/kg encompasses a low dose of IL-2. In certain embodiments, an IL-2 dose is 135×10³ IU/kg encompasses a low dose of IL-2.

In some embodiments, the dose of IL-2 is between about 10×10³ IU/kg-500×10³ IU/kg. In some embodiments, the dose of IL-2 is between about 10×10³ IU/kg-300× 10³ IU/kg. In some embodiments, the dose of IL-2 is between about 15×10³ IU/kg-270×10³ IU/kg. In some embodiments, the dose of IL-2 is about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500×10³ IU/kg. In some embodiments, the dose of IL-2 is about 15×10³ IU/kg. In some embodiments, the dose of IL-2 is about 45×10³ IU/kg. In some embodiments, the dose of IL-2 is about 135×10³ IU/kg. In some embodiments, the dose of IL-2 is about 270×10³ IU/kg. In some embodiments, the dose of IL-2 is about 300×10³ IU/kg. In some embodiments, the dose of IL-2 is about 400×10³ IU/kg. In some embodiments, the dose of IL-2 is about 500×10³ IU/kg. In some embodiments, the dose of IL-2 is 15×10³ IU/kg. In some embodiments, the dose of IL-2 is 45×10³ IU/kg. In some embodiments, the dose of IL-2 is 135×10³ IU/kg. In some embodiments, the dose of IL-2 is 270×10³ IU/kg. In some embodiments, the dose of IL-2 is 300×10³ IU/kg. In some embodiments, the dose of IL-2 is 400×10³ IU/kg. In some embodiments, the dose of IL-2 is 500× 10³ IU/kg.

In some embodiments, methods described herein comprise administering avelumab to a subject at a dose of 240 mg. In other embodiments, methods described herein comprise administering avelumab to a subject at a dose of 120 mg. In other embodiments, methods described herein comprise administering avelumab to a subject at a dose of 360 mg. In other embodiments, methods described herein comprise administering avelumab to a subject at a dose of 600 mg. In other embodiments, methods described herein comprise administering avelumab to a subject at a dose of between about 120-600 mg. In other embodiments, methods described herein comprise administering avelumab to a subject at a dose of between about 500-1000 mg.

In some embodiments, methods described herein comprise administering an anti-IL-2 antibody to a subject at a dose of 9 mg/kg of the subject's body weight, IL-2 at a loading dose of 135,000 IU/kg of the subject's body weight, and IL-2 at a booster dose of 135,000 IU/kg of the subject's body weight. In some embodiments, the methods further comprise the step of administering avelumab at a dose of 800 mg to the subject.

In some embodiments, methods described herein comprise administering an anti-IL-2 antibody to a subject at a dose of 7 mg/kg of the subject's body weight—11 mg/kg of the subject's body weight, IL-2 at a loading dose of 100,000 IU/kg of the subject's body weight—270,000 IU/kg of the subject's body weight, and IL-2 at a booster dose of 100,000 IU/kg of the subject's body weight—270,000 IU/kg of the subject's body weight. In some embodiments, the methods further comprise the step of administering avelumab at a dose of 240 mg per subject-600 mg per subject to the subject.

In other embodiments, methods described herein comprise administering an anti-IL-2 antibody to a subject at a dose of 9 mg/kg of the subject's body weight, IL-2 at a loading dose of 135,000 IU/kg of the subject's body weight, and avelumab at a dose of 800 mg per subject. In some embodiments, the methods further comprise the step of administering IL-2 at a booster dose of 135,000 IU/kg of the subject's body weight to the subject.

In other embodiments, methods described herein comprise administering an anti-IL-2 antibody to a subject at a dose of 7 mg/kg of the subject's body weight—11 mg/kg of the subject's body weight, IL-2 at a loading dose of 100,000 IU/kg of the subject's body weight—270,000 IU/kg of the subject's body weight, and avelumab at a dose of 240 mg per subject—600 mg per subject. In some embodiments, the methods further comprise the step of administering IL-2 at a booster dose of 100,000 IU/kg of the subject's body weight—270,000 IU/kg of the subject's body weight to the subject.

In some embodiments when administering an anti-IL-2 antibody disclosed herein and a single loading dose of IL-2, the dose of anti-IL-2 antibody is 4.5 mg/kg and the dose of IL-2 is 15×10³ IU/kg. In some embodiments when administering an anti-IL-2 antibody disclosed herein and a single loading dose of IL-2, the dose of anti-IL-2 antibody is 4.5 mg/kg and the dose of IL-2 is 45×10³ IU/kg. In some embodiments when administering an anti-IL-2 antibody disclosed herein and a single loading dose of IL-2, the dose of anti-IL-2 antibody is 4.5 mg/kg and the dose of IL-2 is 135×10³ IU/kg. In some embodiments when administering an anti-IL-2 antibody disclosed herein and a single loading dose of IL-2, the dose of anti-IL-2 antibody is 4.5 mg/kg and the dose of IL-2 is 270×10³ IU/kg. In some embodiments when administering an anti-IL-2 antibody disclosed herein and a single loading dose of IL-2, the dose of anti-IL-2 antibody is 4.5 mg/kg and the dose of IL-2 is 300×10³ IU/kg. In some embodiments when administering an anti-IL-2 antibody disclosed herein and a single loading dose of IL-2, the dose of anti-IL-2 antibody is 4.5 mg/kg and the dose of IL-2 is 400×10³ IU/kg. In some embodiments when administering an anti-IL-2 antibody disclosed herein and a single loading dose of IL-2, the dose of anti-IL-2 antibody is 4.5 mg/kg and the dose of IL-2 is 500×10³ IU/kg.

In some embodiments when administering an anti-IL-2 antibody disclosed herein and a single loading dose of IL-2, the dose of anti-IL-2 antibody is 9.0 mg/kg and the dose of IL-2 is 15×10³ IU/kg. In some embodiments when administering an anti-IL-2 antibody disclosed herein and a single loading dose of IL-2, the dose of anti-IL-2 antibody is 9.0 mg/kg and the dose of IL-2 is 45×10³ IU/kg. In some embodiments when administering an anti-IL-2 antibody disclosed herein and a single loading dose of IL-2, the dose of anti-IL-2 antibody is 9.0 mg/kg and the dose of IL-2 is 135×10³ IU/kg. In some embodiments when administering an anti-IL-2 antibody disclosed herein and a single loading dose of IL-2, the dose of anti-IL-2 antibody is 9.0 mg/kg and the dose of IL-2 is 270×10³ IU/kg. In some embodiments when administering an anti-IL-2 antibody disclosed herein and a single loading dose of IL-2, the dose of anti-IL-2 antibody is 9.0 mg/kg and the dose of IL-2 is 300×10³ IU/kg. In some embodiments when administering an anti-IL-2 antibody disclosed herein and a single loading dose of IL-2, the dose of anti-IL-2 antibody is 9.0 mg/kg and the dose of IL-2 is 400×10³ IU/kg. In some embodiments when administering an anti-IL-2 antibody disclosed herein and a single loading dose of IL-2, the dose of anti-IL-2 antibody is 9.0 mg/kg and the dose of IL-2 is 500×10³ IU/kg.

In some embodiments when administering an anti-IL-2 antibody disclosed herein and a single loading dose of IL-2, the dose of anti-IL-2 antibody is 12 mg/kg and the dose of IL-2 is 15×10³ IU/kg. In some embodiments when administering an anti-IL-2 antibody disclosed herein and a single loading dose of IL-2, the dose of anti-IL-2 antibody is 12 mg/kg and the dose of IL-2 is 45×10³ IU/kg. In some embodiments when administering an anti-IL-2 antibody disclosed herein and a single loading dose of IL-2, the dose of anti-IL-2 antibody is 12 mg/kg and the dose of IL-2 is 135×10³ IU/kg. In some embodiments when administering an anti-IL-2 antibody disclosed herein and a single loading dose of IL-2, the dose of anti-IL-2 antibody is 12 mg/kg and the dose of IL-2 is 270×10³ IU/kg. In some embodiments when administering an anti-IL-2 antibody disclosed herein and a single loading dose of IL-2, the dose of anti-IL-2 antibody is 12 mg/kg and the dose of IL-2 is 300×10³ IU/kg. In some embodiments when administering an anti-IL-2 antibody disclosed herein and a single loading dose of IL-2, the dose of anti-IL-2 antibody is 12 mg/kg and the dose of IL-2 is 400×10³ IU/kg. In some embodiments when administering an anti-IL-2 antibody disclosed herein and a single loading dose of IL-2, the dose of anti-IL-2 antibody is 12 mg/kg and the dose of IL-2 is 250×10³ IU/kg.

In some embodiments when administering a combination therapy comprising an anti-IL-2 antibody disclosed herein and IL-2, wherein the dose of anti-IL-2 antibody is between about 0.1 mg/kg and 12 mg/kg anti-IL-2 antibody and the dose of IL-2 is between about 10×10³ IU/kg-300×10³ IU/kg. In some embodiments when administering a combination therapy comprising an anti-IL-2 antibody disclosed herein and IL-2, wherein the dose of anti-IL-2 antibody is between about 0.1 mg/kg and 12 mg/kg anti-IL-2 antibody and the dose of IL-2 is between about 10×10³ IU/kg-500×10³ IU/kg. In some embodiments when administering a combination therapy comprising an anti-IL-2 antibody disclosed herein and IL-2, wherein the dose of anti-IL-2 antibody is between about 0.5 mg/kg and 12 mg/kg anti-IL-2 antibody and the dose of IL-2 is between about 10×10³ IU/kg-300×10³ IU/kg. In some embodiments when administering a combination therapy comprising an anti-IL-2 antibody disclosed herein and IL-2, wherein the dose of anti-IL-2 antibody is between about 0.5 mg/kg and 12 mg/kg anti-IL-2 antibody and the dose of IL-2 is between about 10×10³ IU/kg-500×10³ IU/kg.

In some embodiments when administering a combination therapy comprising an anti-IL-2 antibody disclosed herein and IL-2, the dose of anti-IL-2 antibody is 0.5 mg/kg and the dose of IL-2 is 15×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 45×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 135×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 270×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 300×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 400×10³ IU/kg. In some embodiments when administering a combination therapy comprising an anti-IL-2 antibody disclosed herein and IL-2, the dose of anti-IL-2 antibody is 0.5 mg/kg and the dose of IL-2 is 500×10³ IU/kg.

In some embodiments when administering a combination therapy comprising an anti-IL-2 antibody disclosed herein and IL-2, the dose of anti-IL-2 antibody is 1.5 mg/kg and the dose of IL-2 is 15×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 45×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 135×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 270×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 300×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 400×10³ IU/kg. In some embodiments when administering a combination therapy comprising an anti-IL-2 antibody disclosed herein and IL-2, the dose of anti-IL-2 antibody is 1.5 mg/kg and the dose of IL-2 is 500×10³ IU/kg.

In some embodiments when administering a combination therapy comprising an anti-IL-2 antibody disclosed herein and IL-2, the dose of anti-IL-2 antibody is 4.5 mg/kg and the dose of IL-2 is about 10×10³ IU/kg-300 IU/kg. In some embodiments when administering a combination therapy comprising an anti-IL-2 antibody disclosed herein and IL-2, the dose of anti-IL-2 antibody is 4.5 mg/kg and the dose of IL-2 is about 10×10³ IU/kg-500 IU/kg.

In some embodiments when administering a combination therapy comprising an anti-IL-2 antibody disclosed herein and IL-2, the dose of anti-IL-2 antibody is 4.5 mg/kg and the dose of IL-2 is 15×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 45×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 135×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 270×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 300×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 400×10³ IU/kg. In some embodiments when administering a combination therapy comprising an anti-IL-2 antibody disclosed herein and IL-2, the dose of anti-IL-2 antibody is 4.5 mg/kg and the dose of IL-2 is 500×10³ IU/kg. In some embodiments when administering a combination therapy comprising an anti-IL-2 antibody disclosed herein and IL-2, the dose of anti-IL-2 antibody is 9.0 mg/kg and the dose of IL-2 is about 10×10³ IU/kg-300 IU/kg. In some embodiments when administering a combination therapy comprising an anti-IL-2 antibody disclosed herein and IL-2, the dose of anti-IL-2 antibody is 9.0 mg/kg and the dose of IL-2 is about 10×10³ IU/kg-500 IU/kg.

In some embodiments when administering a combination therapy comprising an anti-IL-2 antibody disclosed herein and IL-2, the dose of anti-IL-2 antibody is 9.0 mg/kg and the dose of IL-2 is 15×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 45×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 135×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 270×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 300×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 400×10³ IU/kg. In some embodiments when administering a combination therapy comprising an anti-IL-2 antibody disclosed herein and IL-2, the dose of anti-IL-2 antibody is 9.0 mg/kg and the dose of IL-2 is 500×10³ IU/kg.

In some embodiments when administering a combination therapy comprising an anti-IL-2 antibody disclosed herein and IL-2, the dose of anti-IL-2 antibody is 12 mg/kg and the dose of IL-2 is 15×10³ IU/kg. In some embodiments when administering a combination therapy comprising an anti-IL-2 antibody disclosed herein and IL-2, the dose of anti-IL-2 antibody is 12 mg/kg and the dose of IL-2 is 45×10³ IU/kg. In some embodiments when administering a combination therapy comprising an anti-IL-2 antibody disclosed herein and IL-2, the dose of anti-IL-2 antibody is 12 mg/kg and the dose of IL-2 is 135×10³ IU/kg. In some embodiments when administering a combination therapy comprising an anti-IL-2 antibody disclosed herein and IL-2, the dose of anti-IL-2 antibody is 12 mg/kg and the dose of IL-2 is 270×10³ IU/kg. In some embodiments when administering a combination therapy comprising an anti-IL-2 antibody disclosed herein and IL-2, the dose of anti-IL-2 antibody is 12 mg/kg and the dose of IL-2 is 300×10³ IU/kg. In some embodiments when administering a combination therapy comprising an anti-IL-2 antibody disclosed herein and IL-2, the dose of anti-IL-2 antibody is 12 mg/kg and the dose of IL-2 is 400×10³ IU/kg. In some embodiments when administering a combination therapy comprising an anti-IL-2 antibody disclosed herein and IL-2, the dose of anti-IL-2 antibody is 12 mg/kg and the dose of IL-2 is 500×10³ IU/kg.

In some embodiments, the dose of IL-2 is considered low when compared with other therapies. In some embodiments, a low dose of IL-2 is between about 10×10³ IU/kg-500×10³ IU/kg. In some embodiments, a low dose of IL-2 is between about 10×10³ IU/kg-500×10³ IU/kg. In some embodiments, a low dose of IL-2 is between about 15×10³ IU/kg-500×10³ IU/kg. In some embodiments, a low dose of IL-2 is between about 45×10³ IU/kg-500×10³ IU/kg. In some embodiments, a low dose of IL-2 is equal to or less than about 500×10³ IU/kg. In some embodiments, a composition comprises an anti-IL-2 antibody comprising anti-IL-2 clone BDG 17.069.

In some embodiments, methods described herein comprise administering an anti-IL-2 antibody to a subject once every 2 weeks, a loading IL-2 dose administered to the subject once, and a booster IL-2 dose administered to the subject as needed. In some embodiments, the booster IL-2 dose is administered to the subject once every 8 weeks. In some embodiments, the methods further comprise the step of administering avelumab to the subject once every 2 weeks.

In some embodiments, methods described herein comprise administering an anti-IL-2 antibody administered to a subject once every 1-4 weeks, a loading IL-2 dose administered to the subject once, and a booster IL-2 dose administered to the subject as needed. In some embodiments, the booster IL-2 dose is administered to the subject once every 4-16 weeks. In some embodiments, the methods further comprise the step of administering avelumab to the subject once every 1-4 weeks.

In other embodiments, methods described herein comprise administering an anti-IL-2 antibody to a subject once every 2 weeks, a loading IL-2 dose administered to the subject once, and avelumab to the subject once every 2 weeks. In some embodiments, the methods further comprise the step of administering IL-2 to the subject as needed. In some embodiments, the booster IL-2 dose is administered to the subject once every 8 weeks.

In other embodiments, methods described herein comprise administering an anti-IL-2 antibody to a subject once every 1-4 weeks, a loading IL-2 dose administered to the subject once, and an immune checkpoint inhibitor, for example avelumab to the subject once every 1-4 weeks. In some embodiments, the methods further comprise the step of administering IL-2 to the subject as needed in a booster dose. In some embodiments, the booster IL-2 dose is administered to the subject once every 4-16 weeks.

As used herein, the terms “booster” or “booster dose” may encompass an additional dose of a therapeutic agent for example IL-2, to enhance or prolong its effect. A skilled artisan would appreciate that a booster dose is a supplementary dose of a therapeutic compound, such as IL-2, given after a loading dose. In some embodiments, the booster dose is administered to provide additional IL-2 to the subject after blood levels of IL-2 are depleted over time. In some embodiments, the anti-IL-2 antibody binds to IL-2 and thereby blocks IL-2 from binding to Treg cells, eosinophils, and pulmonary and vascular endothelial cells expressing the high affinity trimeric receptor (CD25/CD132/CD122). Excess IL-2 that is not complexed to the anti-IL-2 antibody binds to the moderate affinity dimeric receptor (CD132/CD122), which favors the expansion and activation of Teff cells, NK cells, and Natural killer T (NKT) cells.

### Formulations

Pharmaceutical compositions disclosed herein comprising anti-IL-2 antibodies, or a combination of anti-IL-2 antibodies and IL-2, or checkpoint inhibitors, can be conveniently provided as sterile liquid preparations, e.g., isotonic aqueous solutions, suspensions, emulsions, dispersions, or viscous compositions, which may be buffered to a selected pH, Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the anti-IL-2 antibodies, or a combination of anti-IL-2 antibodies and IL-2, or checkpoint inhibitors, described herein and utilized in practicing the methods disclosed herein, in the required amount of the appropriate solvent with various amounts of the other ingredients, as desired. In other embodiments, each component is formulated separately. For example, a sterile injectable solution is prepared for the anti-IL-2 antibody, a sterile injectable solution is prepared for the IL-2, and a sterile injectable solution is prepared for the checkpoint inhibitor, for example but not limited to avelumab. Such formulations may be in admixture with a suitable carrier, diluent, or excipient such as sterile water, physiological saline, glucose, dextrose, or the like. The formulations can also be lyophilized. The formulations can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the formulations, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

In certain embodiments, the terms “pharmaceutical composition”, “composition”, and “formulation” may be used interchangeably having the same meanings and qualities.

The compositions or formulations described herein can be isotonic, i.e., they can have the same osmotic pressure as blood and lacrimal fluid. The desired isotonicity of the compositions as disclosed herein may be accomplished using sodium chloride, or other pharmaceutically acceptable agents such as dextrose, boric acid, sodium tartrate, propylene glycol or other inorganic or organic solutes. Sodium chloride may be preferred particularly for buffers containing sodium ions.

Viscosity of the compositions, if desired, can be maintained at the selected level using a pharmaceutically acceptable thickening agent. Methylcellulose may be preferred because it is readily and economically available and is easy to work with.

Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

In some embodiments, a composition is formulated to be at a pH between about pH 5.0-6.0. In some embodiments, a composition is formulated to be at a pH between about pH 5.0-7.0. In some embodiments, a composition is formulated to be at a pH between about pH 5.0-6.5. In some embodiments, a composition is formulated to be at a pH between about pH 5.0-5.5. In some embodiments, a composition is formulated to be at a pH between about pH 5.5-6.0. In some embodiments, a composition is formulated to be at a pH between about pH 5.5-6.5. In some embodiments, a composition is formulated to be at a pH between about pH 5.0. In some embodiments, a composition is formulated to be at a pH between about pH 5.5. In some embodiments, a composition is formulated to be at a pH between about pH 6.0. In some embodiments, a composition is formulated to be at a pH between about pH 6.5.

In some embodiments, a composition is formulated to be at a pH between about pH 5.0-6.0 and comprises a buffer. In some embodiments, the buffer comprises a pharmaceutically acceptable buffer. In some embodiments, the buffer comprises a histidine buffer or a citrate buffer. In some embodiments, the buffer comprises a histidine buffer. In some embodiments, the buffer comprises a citrate buffer.

In some embodiments, a composition is formulated to be at a pH between about pH 5.0-6.0 and comprises a buffer selected from a histidine buffer and a citrate buffer. In some embodiments, a composition is formulated to be at a pH between about pH 5.0-6.0 and comprises a histidine buffer. In some embodiments, a composition is formulated to be at a pH between about pH 5.0-6.0 and comprises a citrate buffer.

In some embodiments, a composition further comprises at least one of sucrose, methionine, or PS80, or any combination thereof. In some embodiments, a composition further comprises sucrose. In some embodiments, a composition further comprises methionine. In some embodiments, a composition further comprises PS80.

In some embodiments, a composition comprises an anti-IL-2 antibody as disclosed herein and is formulated to be at a pH between about pH 5.0-6.0 and comprises a buffer selected from a histidine buffer and a citrate buffer. In some embodiments, the composition further comprises IL-2.

Those skilled in the art will recognize that the components of the compositions or formulations should be selected to be chemically inert and will not affect the viability or efficacy of the early apoptotic cell populations as described herein, for use in the methods disclosed herein. This will present no problem to those skilled in chemical and pharmaceutical principles, or problems can be readily avoided by reference to standard texts or by simple experiments (not involving undue experimentation), from this disclosure and the documents cited herein.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

A skilled artisan would appreciate that the term “about”, may encompass a deviance of between 0.0001-5% from the indicated number or range of numbers. In some instances, the term “about”, may encompass a deviance of between 1-10% from the indicated number or range of numbers. In some instances, the term “about”, encompasses a deviance of up to 25% from the indicated number or range of numbers.

As used herein, the term “dose” may encompass, in some embodiments, a measured quantity of a therapeutic agent to be taken at one time. A skilled artisan would appreciate that administration of a dose encompasses providing the given measured quantity of the therapeutic agent to a subject.

### EXAMPLES

### Example 1

Phase 1/2 Clinical Trial for AU-007 with Recombinant Human IL-2 (Aldesleukin)

Engineered anti-IL-2 antibodies designed to redirect IL-2 activity toward effector T cells and NK cells while avoiding stimulation of regulatory T cells (Tregs) and CD25+ endothelial cells were generated and characterized as described in US-2024/0287169, which is incorporated herein by reference in its entirety.

AU-007 is a human mAb that binds IL-2 on its CD25 binding epitope. AU-007 bound IL-2 cannot bind trimeric (CD25, CD122, CD132) IL-2 receptors (IL-2R) on regulatory T cells (Tregs), vascular endothelium, or eosinophils, but IL-2's binding to dimeric IL-2R (CD122, CD132) on T effector (T eff) and NK cells is unhindered. AU-007 thus redirects IL-2 towards T eff and NK cell activation, while diminishing Treg activation and vascular leak. AU-007 uniquely redirects IL-2 generated from T eff cell expansion, converting a Treg-mediated autoinhibitory loop into an immune stimulating loop (as described in US-2024/0287169, which is incorporated herein by reference in its entirety). CDR sequences are defined according to IMGT numbering.

The heavy chain variable region and light chain variable region amino acid sequences of Clone 17.069 are presented in Table 2. Clone 17.069 comprises the LALA mutation (L234A, L235A mutations).

TABLE 2
VH and VL Amino Acid Sequences
of Anti-IL-2 Clones.

| | Heavy Chain | Light Chain |
|---|---|---|
| | Variable | Variable |
| | Region | Region |
| Clone | (VH) | (VL) |
| 17.069 | QVQLVQSGAEVKKPG | DIVMTQSPDSLAVSL |
| | SSVKVSCKASGYSIT | GERATINCKSSQSLL |
| | DDLIHWVRQAPGQGL | RRGNQKNHLAWYQQK |
| | EWMGWIDPEDGETNY | PGQPPKLLIYDASTG |
| | AQKFQGRVTLTADTS | QSGVPDRFSGSGSGT |
| | TSTAYMELSSLRSED | DFTLTISSLQAEDVA |
| | TAVYYCARSLDSTWI | VYYCLQSYITPPTFG |
| | YPFAYWGQGTLVTVS | AGTKVEIK |
| | S | (SEQ ID NO: 27) |
| | (SEQ ID NO: 26) | |

The CDR sequences for Clone 17.069 is provided in Table 3.

TABLE 3
CDR Amino Acid Sequences of Anti-IL-2 Clones.

| | Heavy Chain | | | Light Chain | | |
|---|---|---|---|---|---|---|
| Clones | CDR1 | CDR2 | CDR3 | CDR1 | CDR2 | CDR3 |
| 17.069 | GYSI | IDPE | ARSL | QSLL | DAS | LQSYI |
| | TDDL | DGET | DSTW | RRGN | | TPPT |
| | (SEQ | (SEQ | IYPF | QKNH | | (SEQ |
| | ID | ID | AY | (SEQ | | ID |
| | NO: | NO: | (SEQ | ID | | NO: |
| | 62) | 63) | ID | NO: | | 67) |
| | | | NO: | 65) | | |
| | | | 64) | | | |

The full length amino acid sequences for Clone 17.069 is provided in Table 4 below.

TABLE 4
Full-length Amino Acid Sequences
of Anti-IL-2 Clones.

| BGD- | Heavy Chain | |
|---|---|---|
| Clone | (LALA) | Light Chain |
| BDG17.069 | QVQLVQSGAEVKKPG | DIVMTQSPDSLAVSL |
| | SSVKVSCKASGYSIT | GERATINCKSSQSLL |
| | DDLIHWVRQAPGQGL | RRGNQKNHLAWYQQK |
| | EWMGWIDPEDGETNY | PGQPPKLLIYDASTG |
| | AQKFQGRVTLTADTS | QSGVPDRFSGSGSGT |
| | TSTAYMELSSLRSED | DFTLTISSLQAEDVA |
| | TAVYYCARSLDSTWI | VYYCLQSYITPPTFG |
| | YPFAYWGQGTLVTVS | AGTKVEIKRTVAAPS |
| | SASTKGPSVFPLAPS | VFIFPPSDEQLKSGT |
| | SKSTSGGTAALGCLV | ASVVCLLNNFYPREA |
| | KDYFPEPVTVSWNSG | KVQWKVDNALQSGNS |
| | ALTSGVHTFPAVLQS | QESVTEQDSKDSTYS |
| | SGLYSLSSVVTVPSS | LSSTLTLSKADYEKH |
| | SLGTQTYICNVNHKP | KVYACEVTHQGLSSP |
| | SNTKVDKKVEPKSCD | VTKSFNRGEC |
| | KTHTCPPCPAPEAAG | (SEQ ID NO: 73) |
| | GPSVFLFPPKPKDTL | |
| | MISRTPEVTCVVDVS | |
| | HEDPEVKFNWYVDGV | |
| | EVHNAKTKPREEQYN | |
| | STYRVVSVLTVLHQD | |
| | WLNGKEYKCKVSNKA | |
| | LPAPIEKTISKAKGQ | |
| | PREPQVYTLPPSRDE | |
| | LTKNQVSLTCLVKGF | |
| | YPSDIAVEWESNGQP | |
| | ENNYKTTPPVLDSDG | |
| | SFFLYSKLTVDKSRW | |
| | QQGNVFSCSVMHEAL | |
| | HNHYTQKSLSLSPGK | |
| | (SEQ ID NO: 72) | |

AU-007 may be formulated in 20 mM Histidine, 8% sucrose, 10 mM Methionine, 0.04% PS80, pH5.5 (F4).

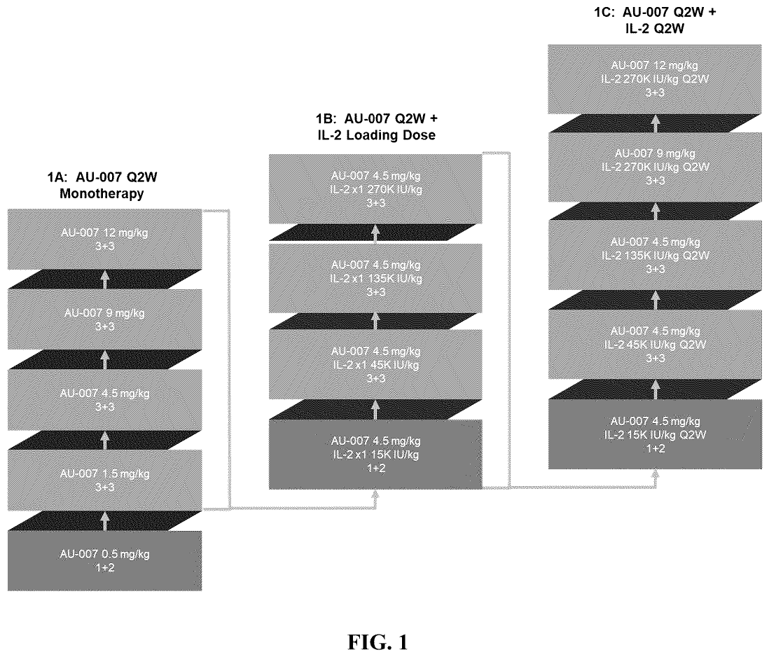

A clinical study was designed to test the efficacy of AU-007 with recombinant IL-2 (aldesleukin) in patients with cancer. In the study, 3 dose escalation arms were followed by cohort expansion (FIG. 1). Arm 1A evaluated escalating doses (0.5-12 mg/kg of said subject's body weight) of AU-007 (IV every 2 weeks [Q2W]). Arm 1B evaluated AU-007 Q2W+a single escalating loading low doses (15K-270K IU/kg of said subject's body weight) of aldesleukin administered subcutaneously (SC). Arm 1C evaluated AU-007+ escalating low doses of SC aldesleukin, both Q2W. There were at least twenty solid tumor types allowed in the dose escalation arms. Cohort expansion Arm 2B evaluated 9 mg/kg of said subject's body weight AU-007+ one loading dose of aldesleukin at a dose of 135K IU/kg of the subject's body weight. Tumor assessments were conducted at the end of each 8-week cycle.

In the B and C Arms (or B and C regimens) of the study, recombinant human IL-2 (aldesleukin) was administered subcutaneously, at much lower doses (15K IU/kg, 45K IU/kg, 135K IU/kg, or 270K IU/kg of said subject's body weight) and much less frequently than the approved regimen of intravenously administered aldesleukin, which is 600,000 IU/kg of said subject's body weight every 8 hours for up to 14 administrations.

In FIG. 1, the terms “3+3” and “1+2” refer to the size of the cohort at a given dose level. For 3+3, the first 3 enrolled patients were or will be administered AU-007 at the dose level that is noted. If no dose-limiting toxicities (DLTs) are seen, then administration may escalate to the next highest dose level. However, if any of the first 3 patients do have a dose-limiting toxicity that is drug-related, then an additional three more patients will receive treatment at that same dose level to see if they have DLTs before escalating. 1+2 follows the same principle-start with one patient, if DLTs are observed, add two more, or if no DLTs are observed in the first single patient, then dosages can be escalated.

The 20 solid tumor histologies of patients enrolled in the trial are:
1. Urothelial cancer arising in the bladder, renal pelvis, ureter, or urethra that has progressed during or following an anti-PDx therapy and, if eligible, a platinum-containing regimen.
2. Adrenocortical carcinoma that is unresectable, locally advanced, or metastatic.
3. Clear cell renal cell carcinoma (ccRCC) progressing during or following at least 2 approved therapeutic regimens (e.g., small molecule inhibitors, anti-PDx therapy).
4. Melanoma that is either locally unresectable or metastatic:
   a. BRAF wt: progressed after receiving anti-PD-1 containing therapy with or without an anti-CTLA-4; or
   b. BRAF mut: progressed after a BRAF+MEK inhibitor.
5. Triple-negative breast cancer that is unresectable locally advanced or metastatic and that is refractory to standard 1^{st} line therapy, which may include cytotoxic chemotherapy alone and/or poly ADP ribose polymerase (PARP) inhibitors for breast cancer gene (BRCA), 1 or 2 mutations, and/or anti-PDx therapy in MSI-H/dMMR positive tumors.
6. Head and neck squamous cell carcinoma (HNSCC) that has progressed during or following treatment with an anti-PDx (unless ineligible, e.g., patients failing chemotherapy and PD-L1 combined positive score (CPS)<1) and platinum-based chemotherapy (unless ineligible for platinum chemotherapy) for metastatic or recurrent disease.
7. Gastric or gastro-esophageal cancer progressing during or after cytotoxic chemotherapy (e.g., paclitaxel, fluoropyrimidine, platinum agents) with or without trastuzumab (for HER2 overexpressing adenocarcinoma) and with or without anti PD-1 inhibitor therapy. Patients with a CPS≥1 should have received an anti PD-1 containing regimen (unless intolerant or therapy unavailable).
8. Esophageal squamous cell carcinoma progressing during or after cytotoxic chemotherapy (e.g., paclitaxel, fluoropyrimidine, platinum agents) with an anti PD-1 therapy. Patients with a CPS≥10 should have received an anti PD-1 containing regimen (unless intolerant or therapy unavailable).
9. Cutaneous squamous cell carcinoma (cSCC): recurrent or metastatic cSCC that is not curable by surgery or radiation.
10. Pancreatic adenocarcinoma that is unresectable locally advanced or metastatic and received at least one line of chemotherapy (e.g., FOLFIRINOX) (unless ineligible or not feasible).
11. Cholangiocarcinoma that is unresectable locally advanced or metastatic in patients who have had ≥1 line of systemic chemotherapy, unless the patient is ineligible for chemotherapy.
12. Hepato-cellular carcinoma (HCC) progressing during or following an approved therapeutic regimen (unless ineligible).
13. Colorectal cancer (CRC):
   a. K-Ras wild type: Patients who have progressed during or after, or are ineligible for, both irinotecan-based and oxaliplatin-based chemotherapy and who are relapsed or refractory to at least 1 prior systemic therapy that included an anti-epidermal growth factor receptor (EGFR) antibody, such as cetuximab or panitumumab; or
   b. K-Ras mutant: Patients who have progressed during or after, or are ineligible for, both irinotecan and oxaliplatin based chemotherapy (±bevacizumab).
14. Epithelial ovarian cancer progressing during or following at least one prior cytotoxic chemotherapeutic regimen (unless ineligible), and subsequent poly ADP ribose polymerase (PARP) inhibitor therapy in BRCA mutation positive patients (unless ineligible).
15. Cervical cancer progressing during or following first-line cytotoxic chemotherapy and second-line cytotoxic chemotherapy or anti-PDx therapy in PD-L1 positive (CPS≥1) or MSI-H/dMMR positive tumors (unless ineligible).
16. Endometrial cancer in patients progressing on or following either cytotoxic chemotherapy (±trastuzumab) or hormone therapy, and an anti-PDx therapy in MSI-H/dMMR positive tumors.
17. Thyroid cancer (follicular or papillary histology) that is iodine refractory.
18. Non-small cell lung cancer (NSCLC) that has progressed during or following treatment with platinum-based chemotherapy and an anti-PDx therapy for unresectable locally advanced or metastatic disease. NSCLC harboring an activating EGFR mutation (excluding Exon 20 insertion mutations) or anaplastic lymphoma kinase (ALK) rearrangement must have progressed following available EGFR or ALK-targeted therapy in addition to treatment with platinum-based chemotherapy (unless ineligible for platinum therapy).
19. Merkel Cell Carcinoma: metastatic Merkel cell carcinoma that is not curable by surgery or radiation.
20. Nasopharyngeal cancer.

In addition to the 20 solid tumor histologies listed above, patients entered into the trial may be suffering any of the solid tumors including a melanoma, a metastatic melanoma, a primary melanoma and metastatic melanoma, a renal cell carcinoma (RCC), a non-small cell lung cancer (NSCLC), a bladder cancer, a head and neck cancer, a head and neck squamous cell carcinoma (HNSCC), a nasopharyngeal carcinoma, a urothelial cancer, an adrenal cortical carcinoma, a clear cell renal cell carcinoma (ccRCC), a triple-negative breast cancer, a gastric or gastro-esophageal cancer, an esophageal squamous cell carcinoma, a cutaneous squamous cell carcinoma (cSCC), a pancreatic cancer, a pancreatic adenocarcinoma, a cholangiocarcinoma (bile duct cancer), a hepato-cellular carcinoma (HCC), a colorectal cancer (CRC), an epithelial ovarian cancer, a cervical cancer, an endometrial cancer, a thyroid cancer (follicular or papillary histology), a lung cancer, a uterine cancer, a gallbladder cancer, or a Merkel cell carcinoma, or any tumors that are microsatellite instabilities (MSI)-high tumors. In some cases, the cancer may be unresectable locally advanced or metastatic cancer.

Adverse events were graded by the Common Terminology Criteria for Adverse Events (CTCAE). Efficacy evaluation was based on PD (Pharmacodynamic) markers of immune stimulation, total IL-2 (bound to AU-007+free IL-2) and objective responses.

Evaluation of efficacy is based on PD markers of immune stimulation, total IL-2 (bound to AU-007+free IL-2), and objective response.

Peripheral blood samples were taken prior to dosing on Day 1 and following dosing at 4 hours, and on Days 2, 3, 15, 29, and 43 of cycle 1 and pre-dose/Day 29 on all other cycles. Dosing with AU-007 was done via intravenous dosing by weight-based dosing (mg/kg). Proleukin® (aldesleukin) was dosed subcutaneously by weight-based dose (IU/kg). Whole blood was stained for CD4+ T cells, CD8+ T cells, CD4+ Tregs, NK cells, B cells, and monocytes. Samples were analyzed by flow cytometry using TruCount™ for absolute cell counts. Changes from baseline values were determined off absolute counts. In addition, differential hematology counts were taken on Days 1, 15, and 29 of all cycles (or as needed per treating physician's decision) for safety evaluation and to examine eosinophil levels (eosinophils express the IL-2 trimeric receptor). Serum was taken prior to dosing on Day 1 and at 2 hours, 6 hours, and Days 2, 3, 15 pre/post, and 6 hours, 29 pre/post, 43 pre/post, and subsequent Day 1 of each cycle at pre/post. Samples were analyzed for INF-γ (LOQ 31 fg/ml), IL-2 (LOQ 31 fg/ml), and sCD25 (10 fg/ml) using the ECL Mesoscale Dynamics platform.

### Example 2

### Clinical Trial Methodology

### Assessments of Subjects During the Screening Period (Pre-Treatment), During the Treatment Period, and Post-Treatment

The Screening Period was conducted within 28 days of Cycle 1 Day 1 and included the following procedures:
Physical examination including height and weight
Vital signs
Performance status
Medical history
Cancer history
Routine blood tests: comprehensive chemistry panel, special chemistry, hematology, coagulation, endocrine test
Tumor markers appropriate for tumor type (e.g., CA-125, CEA, CA19-9, LDH)
Routine urine test
Electrocardiogram
Urine or blood pregnancy test (women of childbearing potential)
CT or MRI scan or ultrasound or positron emission tomography (PET) scan if appropriate
Brain CT or MRI scan if appropriate
PD-L1 testing (if not previously tested)·
Biopsy

The Treatment Period (Day 1 to Day 56 of each cycle) includes the following procedures:
Physical examination
Interim medical history
Study drug administration
Post-treatment observation
Vital signs
Performance status
Routine blood tests: comprehensive chemistry panel, special chemistry, hematology, coagulation, endocrine test
Tumor markers
Research blood tests: pharmacokinetics, cytokines and soluble biomarkers, flow cytometry, anti-drug antibodies
Routine urine test
Electrocardiogram
Urine or blood pregnancy test (women of childbearing potential)
CT or MRI scan or ultrasound or positron emission tomography (PET) scan if appropriate
Brain CT or MRI scan if appropriate
Biopsy Cycle 2 Day 1 (optional in Dose Escalation)

### Post-Treatment Assessments

Following the last dose of study drug, all patients are followed by electronic (e.g., email) or telephone communication for safety and survival at Day 28 and Day 90, and then every 2 months for survival during a one-year Survival Follow-up Period.

### Safety

Dose-limiting toxicity criteria are defined as follows:

### Hematologic Dose-Limiting Toxicity

Grade 4 neutropenia lasting >7 days.
Grade 3 neutropenic fever.
Grade 3 thrombocytopenia with clinically significant bleeding.

### Hepatic Non-hematologic Dose Limiting Toxicity

Grade 3 aspartate aminotransferase (AST) or alanine aminotransferase (ALT) increase >10×the upper limit of normal (ULN).
Grade 3 AST or ALT increase >5.0 to 10.0×ULN and not resolving to Grade 2 (i.e., >3.0 to 5.0×ULN) within 7 days and Grade 1 (i.e., >ULN to 3.0×ULN) within 14 days (unless patient enrolled with elevated transaminases secondary to metastases, then must resolve to baseline). Steroids must be tapered to ≤10 mg of prednisone or equivalent per day by Day 14.
Grade 3 bilirubin increase >5×ULN.
Grade 3 bilirubin increase >3.0 to 5.0×ULN and not resolving to Grade 2 (i.e., >1.5 to 3.0×ULN) within 7 days and Grade 1 (i.e., >ULN to 1.5×ULN) within 14 days. Steroids must be tapered to ≤10 mg of prednisone or equivalent per day by Day 14.
Any event meeting the Hy's law criteria (all 3 features must apply):
   AST and/or ALT>3×ULN.
   Concurrent elevation of total bilirubin >2×ULN.
   No alternative etiology can be identified for combination increases of AST/ALT and total bilirubin.

### Non-Hematologic Dose Limiting Toxicity

Non-hematologic DLTs are Grade ≥3 non-hematologic AEs with the following exceptions:
Grade 3 electrolyte abnormality lasting <72 hours without associated clinical complications and responds to therapy.
Grade 3 fever lasting <72 hours and not associated with hemodynamic instability.
Grade 3 nausea or vomiting resolving to ≤Grade 1 within 72 hours with or without medical intervention.
Grade 3 amylase and/or lipase elevations not associated with either clinical or radiographic evidence of pancreatitis requiring hospitalization.
Grade 3 diarrhea, constipation, abdominal pain, cramping, dyspepsia, or dysphagia resolving to Grade≤1 within 72 hours with or without medical therapy.
Grade 3 fatigue resolving to Grade 1 within <7 days.
Grade 3 CLS.
Grade 3 IRR including cytokine release syndrome lasting≤12 hours and responding to medical intervention. Grading is based on overall IRR event, and not grade of individual signs or symptoms including:
   Fever, chills, nausea or vomiting, diarrhea, hypotension, hypertension, or tachycardia.
Grade 3 endocrinopathy controlled with hormone supplementation.
Grade 3 skin toxicity resolving to Grade≤2 within 7 days with oral corticosteroids.
Grade 3 inflammatory reaction secondary to anti-tumor response (e.g., metastatic sites, lymph nodes) resolving to Grade≤1 within 7 days.

### Grade 2 Non-Hematologic AEs May be Considered DLTs.

Grade 2 AEs that are prolonged inordinately, based upon the medical judgment of the Investigator, and/or lead to permanent discontinuation of AU-007 due to patient intolerance.

Discontinuation criteria include:
Patient meets criteria for objective progression by modified Response Evaluation Criteria In Solid Tumors (RECIST) v1.1, i.e., progression that is confirmed by a second scan after receiving one more cycle of AU-007.
Occurrence of a DLT.
Sponsor, Investigator, or regulatory agency terminates the study.
Withdrawal of patient due to an adverse event or serious adverse event.
Patient experiences a medical condition that requires treatment with prohibited concomitant medication.
Withdrawal of patient consent.
Completion of protocol-defined therapy.
Pregnancy.

Patients who remain clinically stable (per RECIST definition below) and do not experience dose limiting toxicities (DLTs) or unacceptable toxicity during Cycle 1 may continue to receive additional 8-week treatment cycles until discontinuation criteria are met.

### Efficacy

Tumor assessment (or evaluation) (CT scan, MRI, PET scan, or ultrasound) is carried out at the end of each cycle, on Study Day 56. Tumor evaluation refers to imaging (CT scan, MRI, PET scan, or ultrasound) to measure tumors and assess for any new tumors —see RECIST definition below.

Response is based on RECIST v1.1:
Complete Response: Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on the study (this includes the baseline sum if that is the smallest on study). In addition to the relative increase of 20%, the sum must also demonstrate an absolute increase of at least 5 mm. The appearance of one or more new lesions is also considered progression.
Stable Disease (SD): Neither sufficient shrinkage to qualify for PR nor sufficient increase to qualify for PD, taking as reference the smallest sum diameters while on study.

Exploratory biopsies are optionally conducted to evaluate the effect of the treatment on immune cell infiltration within the tumor. Biopsies are not conducted as part of determining a patient's response to treatment in conjunction with CT scans.

Patient blood samples for cytokines and soluble biomarkers were obtained per the following schedule:

### Cycle 1:

Day 1 pre-dose (prior to first study drug administration on a given day) and 2 and 6 hours post-dose
Day 2
Day 3
Day 15 pre-dose, end of infusion, and 6 hours post-dose
Day 29 pre-dose and end of infusion
Day 43 pre-dose and end of infusion

### Cycle 2:

Day 1 pre-dose and end of infusion
Day 15 pre-dose and end of infusion
Day 43 pre-dose and end of infusion

### Cycles 3 and Beyond:

Day 1 pre-dose and end of infusion.

### Example 3

### Dose Selection for Phase 2 Trials (Recommended Phase 2 Dose; RP2D)

### Methods:

The assay below examines safety assessments conducted under the clinical protocol. Pharmacokinetics (PK) assays, and pharmacodynamic (PD) assays were carried out for the B regimen (Arm B) and C regimen (Arm C) to identify the doses to be used in Phase 2 Expansion Cohorts.

Pharmacokinetic data were derived using standard antibody PK techniques using a capture electrochemolumenescent (ECL) assay by coating a mouse anti-AU-007 antibody on an ECL plate followed by incubating with serum. Detection was done using sulfa-Tagged anti-AU-007 (different from capture).

Pharmacodynamic measures included changes in circulating cell populations and peripheral interferon gamma (IFN-γ) expression.

Circulating cells populations (CD8+T and NK effector cells; and Treg Cells) were determined by flow cytometry and peripheral blood cytokines (IL-2, and IFN-γ), and sCD25 were determined using electrochemiluminescence (SPLEX and VPlex kits; https://www.mesoscale.com/en/products_and_services/assay_kits).

Data from the assays were collated and compared across dosing regimens and levels to determine the dose to be used in the expansion.

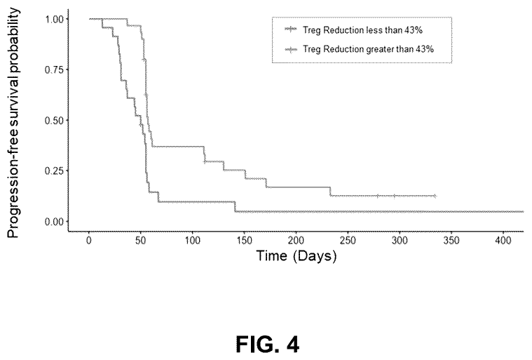

To assess the level of correlation between progression free survival (PFS) and Treg reduction, the largest reduction achieved after Day 3 of treatment was calculated for each participant. Patients were then grouped by those who achieved less than the median Treg reduction and those who achieved more than the median Treg reduction, and PFS was plotted on a Kaplan-Meier curve (FIG. 4). This allows determination of whether a larger Treg reduction is linked to better clinical outcomes, providing evidence for the immunological effects of treatment and their relationship to disease progression.

Progression-free survival (PFS) is defined as the number of days between the first dose of study drug and the first occurrence of objective progressive disease or death, whichever occurs first. Data from patients without progression or death at the time of the last tumor assessment were analyzed in accordance with acceptable censoring techniques. Censoring is an approach to account for incomplete data at the time of the analysis. For example, there may be patients who are in between visits for determining their tumor measurements, or their vital status (alive or dead), in which case the data point used for the PFS analysis would be the last time they had a tumor assessment so you are including the last known data point.

A Cox proportional hazards model of PFS was utilized to evaluate the risk of progression by dose. The model includes effects for AU-007 dose, aldesleukin dose, aldesleukin regimen (B or C Regimen), disease type (RCC, melanoma, or other), and other biomarkers.

The plots show the estimated relative risk (aka hazard ratio) of progression or death for each combination of aldesleukin dose, AU-007 dose, and disease (holding other variables constant). The reference group is a single aldesleukin dose of 135,000 IU/kg and AU-007 dose of 4.5 mg/kg in the “other” disease type, so the plotted values are indexed against that group.

### Results

### Pharmacokinetics Results

Table 5 presents PK data showing the pharmacokinetic data of patients based on AU-007 dosage and administration regime (Arm A, B, or C).

TABLE 5
AU-007 Pharmacokinetic Profile.

| | AU-007 Dose | | Cmax | AUClast |
|---|---|---|---|---|
| Arm | (mg/kg) | N | (mg/mL) | (d * mg/mL) |
| 1A* | 0.5 | 2 | 10.8 (16) | 53.7 (105) |
| 1A | 1.5 | 3 | 29.6 (13) | 231 (12) |
| 1A | 4.5 | 3 | 110 (15) | 828 (42) |
| 1A | 9 | 4 | 255 (21) | 1,700 (22) |
| 1A | 12 | 3 | 282 (9.1) | 1,910 (39) |
| 1B ** | 4.5 | 12 | 132 (47) | 639 (39) |
| 1C *** | 4.5 | 21 | 155 (42) | 773 (46) |
| 1C | 9 | 4 | 223 (12) | 1,350 (25) |

*Arm 1A—AU-007 Q2W-monotherapy
**Arm 1B— AU-007 Q2W + IL-2 loading dose − 15K; 45 K; 135 K; 270K IU/kg
***Arm 1C—AU-007 Q2W + IL-2 Q2W − 15K; 45 K; 135 K; 270K IU/kg

The AUC and Cmax were both approximately dose-proportional over the dose range tested (Table 5). The characteristics were typical of an IgG1-LALA monoclonal antibody, with an approximate 15-day half-life in humans. In general, comparable AU-007 PK exposure (AUC) occurred when dosed alone (Arm 1A) or in combination with aldesleukin (Arms 1B and 1C). Table 5 presents PK data showing the pharmacokinetic data of patients based on AU-007 dosage and administration regime (Arm A, B, or C).

### Table

There was no evidence of neutralizing anti-drug antibodies. These data demonstrate that AU-007 with or without IL-2 combination demonstrates low potential for immunogenicity.

### Peripheral Cell Populations

The mean fold change in peripheral cell populations on Day 43 following treatment initiation, regardless of AU-007 dose (by evaluating Arm B patient samples only-AU-007 4.5 mg/kg) was evaluated at each aldesleukin dose level (Table 6).

Day 43 was the end of Cycle 1 lab collection timepoint and was an adequate timeframe to determine changes.

TABLE 6
Mean Fold Change* in Effector cells (CD8 and NK cells) and
Tregs at Day 43

| IL-2 Dose | | CD8 | NK | Treg | CD8/Treg |
|---|---|---|---|---|---|
| Level | N | Cells | Cells | Cells | Ratio |
| 15K IU/kg | 1 | 0.86 | 0.56 | 0.42 | 2.05 |
| 45K IU/kg | 4 | 0.76 | 0.82 | 0.45 | 1.45 |
| 135K IU/kg | 2 | 1.61 | 2.11 | 0.81 | 1.97 |
| 270K IU/kg | 2 | 0.99 | 1.31 | 0.56 | 1.95 |

*Fold change was determined relative to the Day 1 pre-dose sample.

The largest fold change in CD8, NK, and Treg cells on day 43 was seen at the 135,000 IU/kg dose level, with the highest expansion of CD8+T and NK effector cells and a CD8/Treg ratio of 1.97-fold increase (Table 6). These results suggest that concentrations greater than 135K IU/kg gain no further peripheral blood pharmacodynamic advantage.

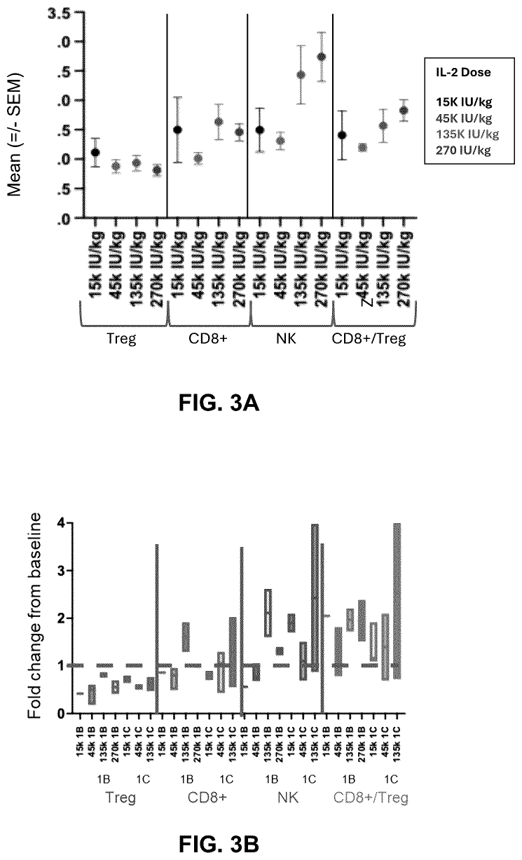

The fold change in peripheral cell population (Treg, NK, CD8+ cell populations, and the CD8/Treg ratio) was evaluated on Day 15 after a first dose of 4.5 mg/kg AU-007 and escalating doses of aldesleukin (FIG. 3A). The Treg population did not change or was decreased relative to baseline (relative to the Day 1 pre-dose sample) when measured on Day 15. Ascending doses of aldesleukin tended to increase CD8+ T cells and NK cells and favored a higher CD8/Treg ratio, with maximum effect seen at 135,000 IU/kg aldesleukin (FIG. 3A).

Day 43 levels of Treg, NK, CD8+ cell populations, and the CD8/Treg ratio in the aldesleukin Dose Escalation cohorts treated with 4.5 mg/kg AU-007 and 15,000 IU/kg, 45,000 IU/kg, 135,000 IU/kg (Arm 1B and 1C), or 270,000 IU/kg (Arm 1B) aldesleukin were also assessed. For all doses, the Treg population was decreased relative to baseline (relative to the Day 1 pre-dose sample). Ascending doses of subcutaneous aldesleukin tended to increase CD8+ T cells and NK cells and favored a higher CD8/Treg ratio (FIG. 3B).

The significance of decreased Treg levels on the duration of progression-free survival (PFS) was examined. The median Treg reduction across Phase 1 and 2 patients was 43% (analysis included patients from Phase 1 and 2 with available pre and post samples at the time of analysis (not including Arm A)). A comparison of PFS between patients with less than 43% reduction in Tregs vs. greater than 43% reduction in Tregs showed that patients with a Treg cell reduction of greater than the median of 43% is associated with longer PFS across Phase 1 and 2 than those with lower Treg reduction (FIG. 4).

### Circulating Interferon Gamma (IFN-γ)

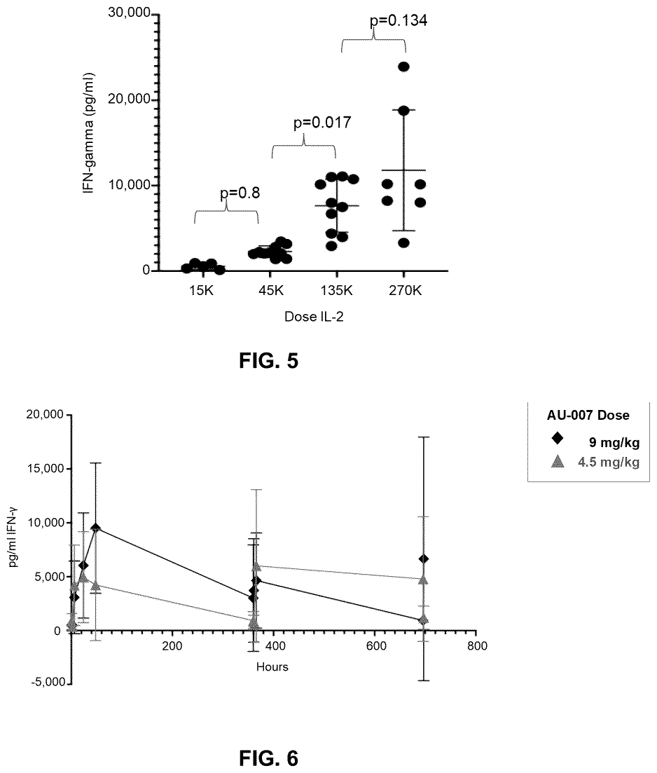

The effect of subcutaneous aldesleukin dose escalation (FIG. 5) or AU-007 dose (FIG. 6) on the concentration of circulating IFN-γ was examined using the ECL Mesoscale Dynamics platform.

Circulating IFN-γ from serum samples generally rose with increasing doses of aldesleukin; however, no significant differences were observed between 135,000 IU/kg and 270,000 IU/kg aldesleukin.

Changes in circulating IFN-γ levels were also observed across both AU-007 dose levels (4.5 mg/kg and 9.0 mg/kg). IFN-γ levels were higher in the first two weeks after dosing with 9 mg/kg AU-007 plus 135,000 IU/kg aldesleukin compared to dosing with 4.5 mg/kg AU-007 plus 135,000 IU/kg aldesleukin (FIG. 6).

A Cox proportional hazards model of PFS was utilized to evaluate the risk of progression by dose. The model includes effects for AU-007 dose, aldesleukin dose, aldesleukin regimen (B or C), disease type (RCC, melanoma, or other), and other biomarkers (FIG. 7).

The plots show the estimated relative risk (aka hazard ratio) of progression or death for each combination of aldesleukin dose, AU-007 dose, and disease (holding other variables constant). The reference group is a single aldesleukin dose of 135,000 IU/kg and AU-007 dose of 4.5 mg/kg in the “other” disease type, so the plotted values are indexed against that group.

Based on modeling of data from the study, dosing with 9 mg/kg AU-007 was associated with a lower risk of disease progression compared to 4.5 mg/kg AU-007 (FIG. 7). The differences between the risk of progression as modeled for each dose level was more distinct in the Arm C cohorts.

The IFN-γ results show that a combination of 9 mg/kg AU-007 plus 135,000 IU/kg aldesleukin is optimal at enhancing IFN-γ production (FIG. 6) with a lower relative risk of progression (FIG. 7) as compared to a combination of 4.5 mg/kg AU-007 plus 135,000 IU/kg aldesleukin.

### Additional Data Supporting AU-007 Dose Selection

There was no substantial safety difference observed across all the AU-007 doses evaluated. All doses demonstrated mostly mild events manageable with over-the-counter medications (data not shown).

There were also no definitive differences in efficacy signals between the 4.5, 9 and 12 mg/kg dose levels.

Preclinical cynomolgus monkey TK predicted that even the second dose level evaluated in escalation (1.5 mg/kg AU-007) would bind >1000-fold the projected IL-2 serum concentrations achieved with the 270K IU/kg aldesleukin

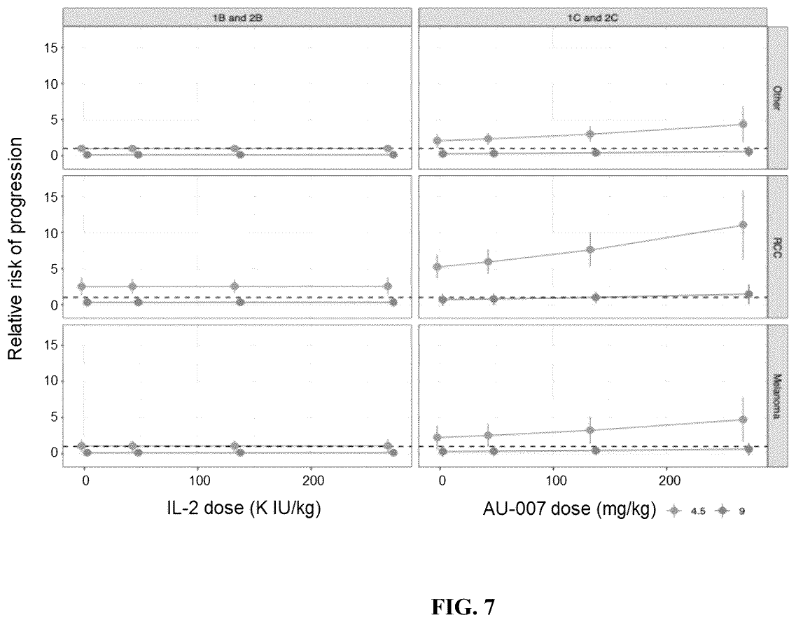

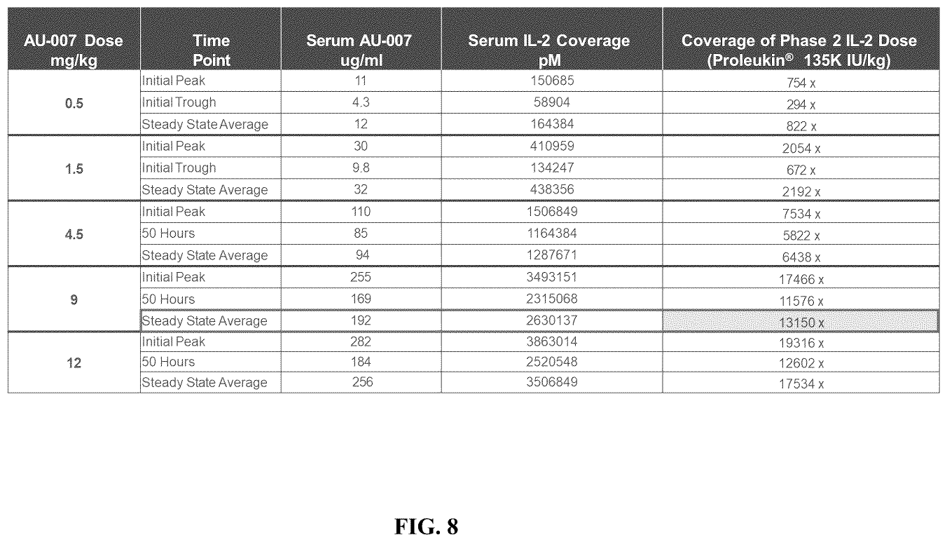

As there were no safety or efficacy differences between the doses, the AU-007 RP2D was selected based on AU-007 coverage of IL-2 molecules (FIG. 8). AU-007 achieves its maximal efficacy with the lowest amounts of unbound IL-2 (endogenous and exogenously administered) in a patient's body. Unbound IL-2 preferentially binds to the trimeric IL-2R found on Tregs (100-fold higher affinity of the trimeric IL-2R for IL-2 vs the dimeric IL-2R) and thus would increase the Treg population.

Therefore, we targeted a 10,000-15,000 overage of AU-007 to IL-2 (135,000 IU/Kg) on a molecule-to-molecule ratio, and the 9 mg/kg AU-007 dose was calculated to provide a ˜13,000-fold overage of AU-007 to IL-2 administered at 135,000 IU/Kg.

In view of the above results, 9 mg/kg AU-007 was selected for the RP2D.

### CONCLUSIONS

The AU-007 PK data demonstrated dose-proportionality over the dose range tested, showed no signs of neutralizing ADA activity, and have an estimated initial T½ Of approximately 15 days in humans (US-2024-0287169, incorporated herein by reference).

The data on escalating doses of aldesleukin demonstrate that higher aldesleukin levels increased peripheral IFN-γ levels and circulating effector cell populations, with the greatest benefit achieved at 135,000 IU/kg of aldesleukin.

AU-007 at all dose levels, regardless of aldesleukin dosing regimen decreased Treg cells in the periphery. Greater reductions in Tregs were associated with longer PFS.

Taken together, the data showed that AU-007 can control and redirect the endogenously produced IL-2 and the exogenously administered IL-2 to reduce the Treg population and increase peripheral IFN-γ levels and circulating CD8+T and NK effector cell populations.

Dosing with 9 mg/kg of AU-007 was optimal at enhancing IFN-γ production (FIG. 6). In addition, the 9 mg/kg AU-007 dose was calculated to provide a ˜13,000-fold coverage of AU-007 to IL-2 administered at 135,000 IU/Kg (FIG. 8).

Based on these results, 9 mg/kg AU-007 Q2W and 135,000 IU/kg subcutaneous aldesleukin with both a single loading aldesleukin dose (2B) and Q2W aldesleukin (2C) schedules were evaluated in the Phase 2 expansion cohorts.

### Example 4

### Regimen Selection for Aldesleukin (IL-2) Administration

### Methods

Data from patients in Arm B and Arm C regimens of Phase 1 or Phase 2 clinical trial were compared to determine the best regimen for administration of AU-007 plus aldesleukin:
Arm B regimen: AU-007 Q2W plus a single loading dose of aldesleukin; and
Arm C regimen: AU-007 Q2W plus aldesleukin Q2W.

Melanoma and renal cell carcinoma (RCC) patients were initially selected for Phase 2 Cohort Expansion.

TABLE 7
summarizes the number of melanoma and renal cell carcinoma (RCC)
patients who received the Arm B regimen or the Arm C regimen:

| | Arm B Regimen | Arm C Regimen |
|---|---|---|
| | AU-007 Q2W + | AU-007 Q2W + |
| | Single Dose | Aldesleukin |
| Diagnosis | Aldesleukin (n) | Q2W (n) |
| Melanoma | 7 | 6 |
| Renal Cell Carcinoma | 5 | 8 |

### Pharmacodynamic Methods

Patient blood samples for cytokines and soluble biomarkers were obtained per the schedule described in Example 2 above. Circulating cells were determined by flow cytometry, and peripheral blood cytokines were determined using electrochemiluminescence. To assess the level of correlation between PFS and Treg reduction, the largest reduction achieved after Day 3 was calculated for each participant. Patients were then grouped by those who achieved less than the median Treg reduction and those who achieved more than the median Treg reduction, and PFS was plotted on a Kaplan-Meier curve (FIG. 13).

### Comparison of Efficacy Results

### Melanoma Patients

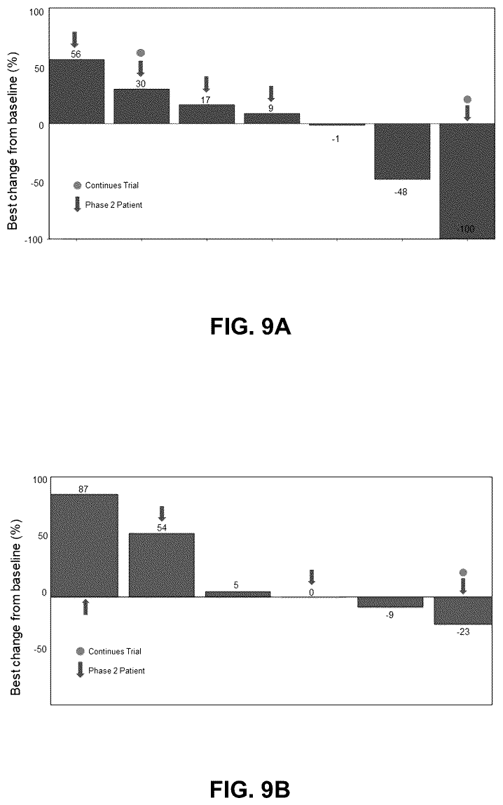

Greater reductions in target tumor size and longer durations of response were seen in melanoma patients receiving the Arm B regimen (AU-007 Q2W plus a single aldesleukin dose), with two patients achieving a 48% and 100% reduction (FIG. 9A) as compared to the Arm C regimen (AU-007 Q2W+SC Aldesleukin Q2W) with two patients achieving a 9% and 23% reduction (FIG. 9B).

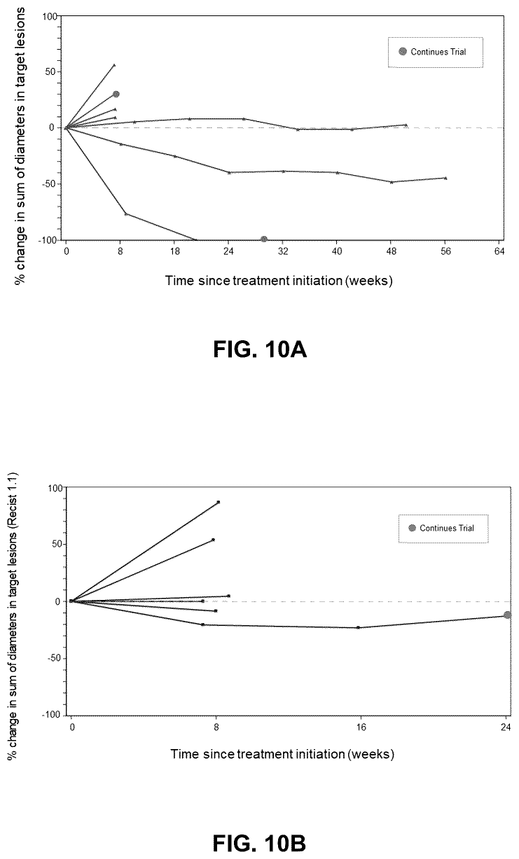

The percentage change in the sum of diameters in target lesions over time (vs. baseline) in Melanoma patients in Phase 1 and Phase 2 patients receiving AU-007 Q2W+ single SC aldesleukin loading dose (Arm B Dosing Regimen; FIG. 10A) and in patients receiving AU-007 Q2W+SC aldesleukin Q2W (Arm C Dosing Regimen; FIG. 10B) were evaluated.

Among melanoma patients receiving AU-007 Q2W plus a single aldesleukin dose, response was maintained to 50 and 56 weeks, with one patient continuing on treatment at approximately Week 30 (FIG. 10A). Among melanoma patients receiving AU-007 plus aldesleukin Q2W, most patients discontinued at Week 8 (end of Cycle 1); one patient continues on treatment at Week 24 (FIG. 10B).

### Renal Cell Carcinoma (RCC) Patients

Target tumor size changes were similar for RCC patients in the Arm B and Arm C regimens, with longer duration of response in one Arm B patient (32 weeks at data cut-off) compared to the Arm C patients (data not shown; one discontinued at Week 8 (−22% change) and one was ongoing at Week 16 [−7% change]).

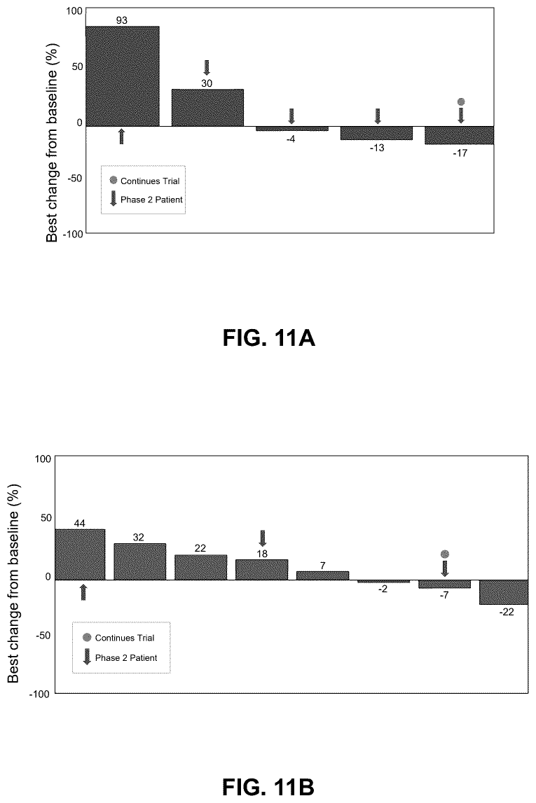

FIG. 11A and FIG. 11B show best response in RCC patients in Phase 1 and Phase 2 receiving AU-007 Q2W+ single SC aldesleukin Loading Dose (Arm B Dosing Regimen) and AU-007 Q2W+SC aldesleukin Q2W (Arm C Dosing Regimen), respectively.

Target tumor reductions of 4%, 13%, and 17% were documented in RCC patients receiving AU-007 plus a single aldesleukin dose (FIG. 11A) as compared to target tumor reductions of 2%, 7%, and 22% documented in RCC patients receiving AU-007 plus aldesleukin Q2W (FIG. 11B).

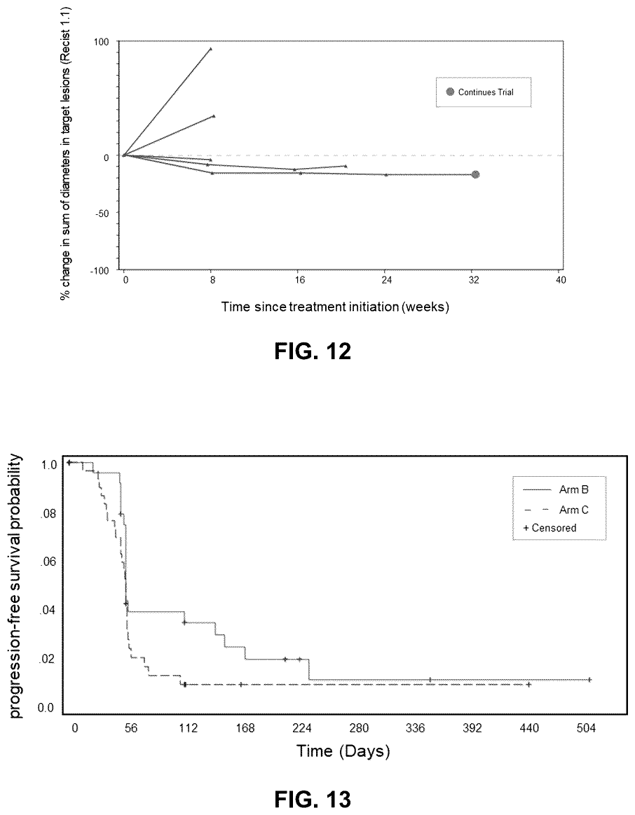

The percentage change in the sum of diameters in target lesions over time (vs. baseline) for RCC patients receiving AU-007 Q2W+ single SC aldesleukin loading dose (Arm B Dosing Regimen; FIG. 12). One patient receiving AU-007 Q2W plus a single aldesleukin dose continues on treatment at Week 32.

Progression-free survival (PFS) was evaluated for all patients (all tumor types) receiving Arm B (n=23) vs. Arm C (n=39) regimen in Phase 1 and 2 (FIG. 13). A Kaplan-Meier plot comparing preliminary PFS data from the Arm B vs. Arm C dosing regimens, across all patients in Phase 1 and Phase 2, demonstrated a trend to longer PFS for patients receiving the Arm B dosing regimen (FIG. 13). Most patients with progression or death at 56 days (end of Cycle 1) were from the Phase 1 dose escalation portion of the study consisting mostly of heavily pre-treated gastrointestinal cancers or cancers not typically associated with response to immunotherapies (e.g., uterine, thyroid cancers).

### Pharmacodynamic Results

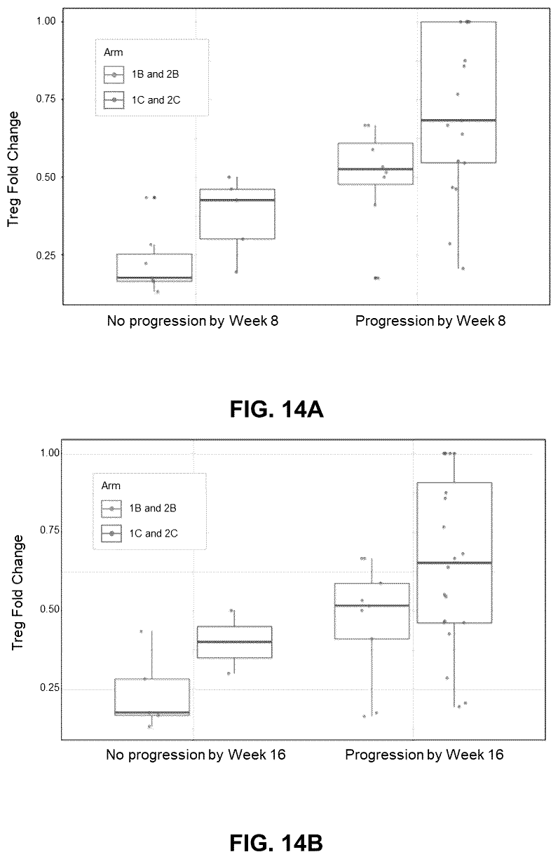

As described above, FIG. 4 demonstrates that Treg cell reduction is associated with longer PFS. The association between Treg cell reduction and longer PFS per dose regimen was evaluated at Week 8 (end of Cycle 1; FIG. 14A) and Week 16 (end of Cycle 2 FIG. 14B). The decrease in Tregs (excluding the first 2 days on treatment) was expressed as a fold change vs. baseline (e.g., 0.25 is a 75% decrease). The results demonstrated that patients with greater Treg decreases were less likely to have a progressed status in Cycles 1 and 2 for both Arm B and Arm C regimens (FIGS. 14A-14B).

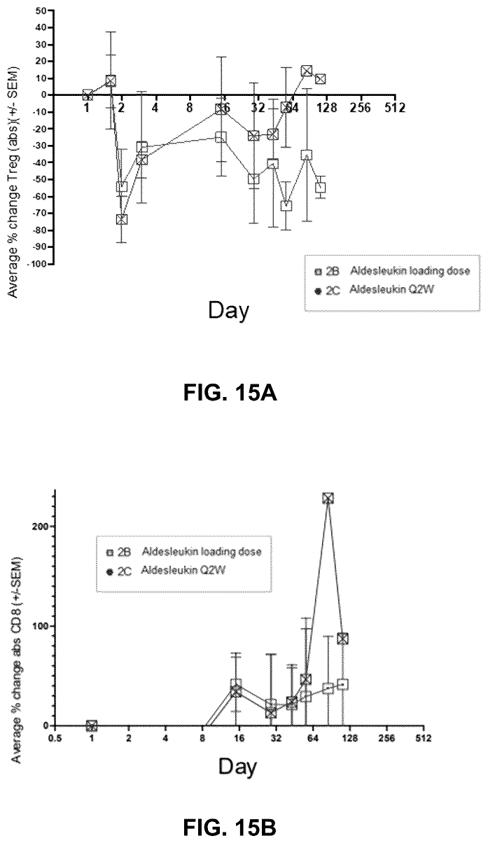

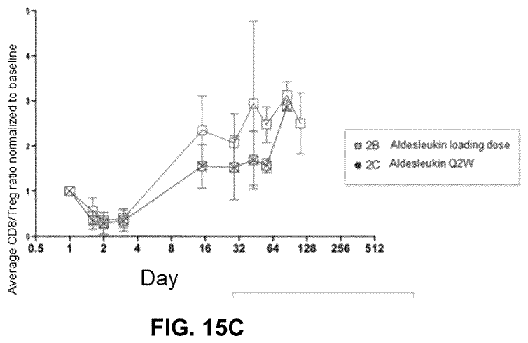

The percent changes in Tregs (FIG. 15A) and CD8 cells (FIG. 15B), and the CD8/Treg ratio (FIG. 15C) in Arm 2B compared to Arm 2C regimens were evaluated. Peripheral Tregs decreased with both Arm 2B and 2C dosing regimens (FIG. 15A), with Arm 2B dosing trending toward greater and more durable decreases of Tregs. Peripheral CD8 cells (FIG. 15B) demonstrated similar increases for Arm B and C dosing regimens, ranging from 30-50% higher than baseline.

The CD8/Treg ratio (FIG. 15C) trended higher for the Arm 2B dosing regimen, since the B and C regimens increased CD8 cells to the same extent while the Treg decrease was greater on the B dosing regimen.

### CONCLUSIONS

Decrease in Tregs appears to be a critical determinant of observed efficacy, with greater decreases in patients receiving a SC aldesleukin loading dose compared to SC aldesleukin Q2W. Therefore, the single SC aldesleukin loading dose regimen (Arm B dosing) was chosen as the regimen for further clinical development based on the following factors:
The additional IL-2 administration with the Arm C regimen did not improve efficacy vs. the Arm B regimen's single SC aldesleukin loading dose (FIGS. 9A-9B and 11A-11B), and patients on the Arm B regimen trend toward having deeper and more durable tumor shrinkage with prolonged PFS (FIGS. 10A-10B).
The Arm B regimen had a strong trend to deeper and more durable Treg decreases (FIGS. 14A-14B and 15A) that are associated with longer PFS in early data (FIG. 13), leading to a greater CD8/Treg ratio.
More prolonged IL-2 exposure on the Arm C dosing regimen may be driving the Effector T (Teff) cells to exhaustion.
The Arm C dosing regimen caused greater and more prolonged increases of interferon-gamma (IFN-γ) vs. the Arm B regimen (data not shown).
Prolonged exposure to IFN-γ may be immune suppressive.

### Example 5

### Combination Therapy Including AU-007, an IL-2 (Aldesleukin) Loading Dose and Avelumab

Patients with unresectable locally advanced or metastatic, PD-L1-positive (tumor proportion score [TPS]≥1%), non-small cell lung cancer (NSCLC) not harboring an activating EGFR mutation or ALK rearrangement that has progressed during or following treatment with an anti-PDX (either PD-1 or PD-L1) with or without platinum-based chemotherapy are eligible for enrollment in the clinical trial of AU-007 in combination with aldesleukin and avelumab (“Part 3” of the study).

Unresectable locally advanced tumors indicate tumors that cannot be resected and have spread locally (compared to metastatic tumors that have spread from the original location to another part of the body).

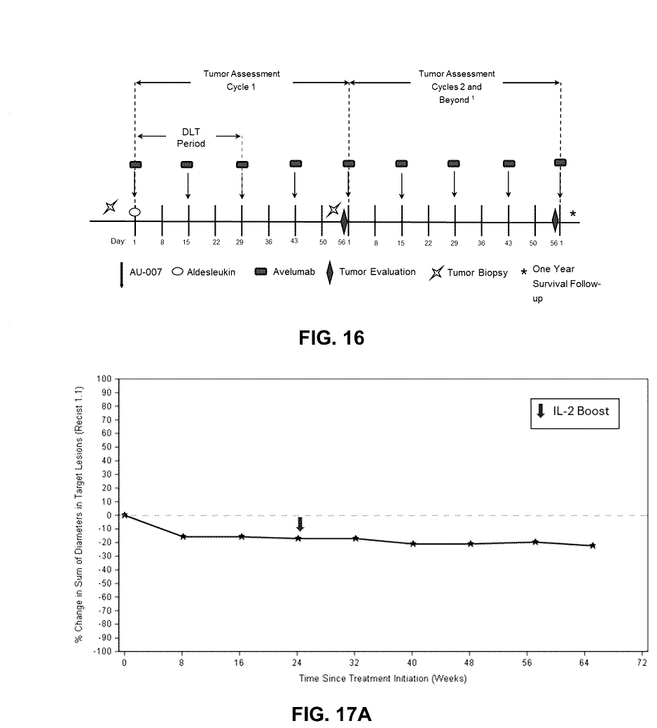

For the combination therapy of: i) AU-007; ii) aldesleukin (IL-2); and iii) avelumab, AU-007 and aldesleukin (IL-2) loading dose are administered at the recommended Phase 2 dose (RP2D) dose (9 mg/kg AU-007 of a subject's body weight (administered IV Q2W) plus a loading dose of 135,000 IU/kg aldesleukin (IL-2) of a subject's body weight (administered SC), as described in Examples 3-4). Avelumab is administered IV at a dose of 800 mg with the initial dose of AU-007 plus aldesleukin and then Q2W. Booster IL-2 doses will be administered if assessment at the time of tumor evaluation warrants (FIG. 16). Assessment for administration of an IL-2 booster dose includes but is not limited to if tumor volume is unchanged, if previously shrinking tumors are stable, if there is an increase in tumor markers, if there is an increase in tumor growth, if new tumor growth is detected in a tumor that had previously been stable or had decreased in size, metastasis, or any combination thereof. An IL-2 booster dose would be administered subcutaneously.

FIG. 16 provides a timeline of the treatment and tumor assessment cycles for Study 3, which examines the effect of combination treatment of AU-007, aldesleukin, and avelumab. Cycles are 8 weeks (56 days). AU-007 is administered 4 times in a cycle (Day 1, 15, 29, 43—see black arrow); Aldesleukin is administered as a loading dose once in Cycle 1 (Day 1—see ellipse); and avelumab is administered 4 times in a cycle (Day 1, 15, 29, 43 —see rectangle).

For patients who continued to subsequent cycles, they began with AU-007 and avelumab on Cycle 2 Day 1, then AU-007 administered with avelumab on Days 15, 29, and 43 of Cycle 2; and so on.

Tumor evaluation (FIG. 16, black diamond) is carried out at the end of a cycle. Tumor evaluation refers to imaging (CT scan, MRI, PET scan, or ultrasound) to measure tumors and assess for any new tumors-see RECIST definition in Example 2.

Biopsy (FIG. 16, 4-point star) is done as an exploratory objective to evaluate immune-modulating effect of drug within tumor by evaluating immune cell infiltration. They are not done as part of determining a patient's response to treatment in conjunction with CT scans.

The Dose-Limiting Toxicity (DLT) period lasted 4 weeks and informed dose escalation decisions. All patients treated in the safety run-in were followed for 4 weeks and their safety assessed before moving to the next dose level.

Patients who remain clinically stable (per RECIST definition in Example 2) and do not experience DLTs or unacceptable toxicity during Cycle 1 may continue to receive additional 8-week treatment cycles with AU-007 plus avelumab until discontinuation criteria as disclosed in Example 2 are met.

The study began with a Screening Period within 28 days of Cycle 1 Day 1 during which the Screening Period procedures described in Example 2 were conducted. The next period of the study was the Treatment Period from Day 1 to Day 56 of each cycle, during which the Treatment Period procedures described in Example 2 were conducted. Finally, Post-Treatment Assessments were conducted following the last dose of study drug. For Post-Treatment Assessments, all patients were followed by electronic (e.g., email) or telephone communication for safety and survival at Day 28 and Day 90, and then every 2 months for survival during a one-year Survival Follow-up Period.

### Safety Run-In Cohorts

An initial safety run-in of the combination therapy was conducted as a 3+3 dose escalation cohort (Table 8) in the Dose Limiting Toxicity (DLT) Evaluation Period (FIG. 16). AU-007+ aldesleukin in combination with avelumab was evaluated starting with the RP2D −1 of AU-007+aldesleukin (Cohort 1 in Table 8 2^{nd} row), in which an IL-2 loading dose of 45,000 IU/kg was used as well as 9 mg/kg AU-007 IV Q2W, and 800 mg avelumab IV Q2W.

TABLE 8
Part 3 Safety Run-In Dose Levels

| | Dose Evaluated | | |
|---|---|---|---|
| Cohort (n) | Aldesleukin | AU-007 | avelumab |
| Dose | Loading Dose | Q2W | Q2W |
| Description | (IU/kg) | (mg/kg) | (mg) |
| −1 | 15,000 | 9 | 800 |
| (3 + 3) | | | |
| RP2D − 2 + | | | |
| Avelumab | | | |
| 1 | 45,000 | 9 | 800 |
| (3 + 3) | | | |
| RP2D − 1 + | | | |
| Avelumab | | | |
| 2 | 135,000 | 9 | 800 |
| (3 + 3) | | | |
| RP2D + | | | |
| Avelumab | | | |

### 3+3 Dose Escalation Cohort: Avelumab

If a DLT as defined in Example 2 was observed during the ‘DLT evaluation period’ (FIG. 16) in any of the initial 3 patients enrolled, 3 patients were added. If two or more DLTs were observed in the initial 6 patients, then the RP2D-2 (Cohort-1) were evaluated in combination with avelumab using an IL-2 loading dose of 15,000 IU/kg.

If no DLTs were observed at the RP2D-1 dose level, a cohort of 3 patients were evaluated at the RP2D+avelumab (Cohort 2) as described for the first cohort.

If no further DLTs were observed, then the Cohort Expansion proceeded with the AU-007+aldesleukin at the recommended Phase 2 dose (RP2D) dose (9 mg/kg AU-007 (administered IV Q2W) plus a loading dose of 135,000 IU/kg aldesleukin (administered SC), as described in Examples 3-4) and avelumab. avelumab was administered IV at a dose of 800 mg with the initial dose of AU-007 plus aldesleukin and then Q2W.

Avelumab dose escalation decisions were driven by clinical safety and all available pharmacokinetic (PK) and pharmacodynamic (PD) data during the dose limiting toxicity (DLT) Evaluation Period, defined as the time between the day of the initial dose up to the day of the 3^{rd} dose administration of AU-007 (planned Study Day 29 of Cycle 1; FIG. 16).

No DLTs were observed in Cohorts 1 and 2. Therefore, Cohort-1 was not initiated. Upon completion of the safety run-in, enrollment of the Cohort Expansion at the RP2D plus avelumab was initiated and is ongoing.

### Cohort Expansion

Patients enrolled in the Part 3 Cohort expansion receive the RP2D dose of 9.0 mg/kg AU-007 Q2W plus a loading dose of 135,000 IU/kg aldesleukin in combination with 800 mg avelumab Q2W as described in FIG. 16.

Patients who remain clinically stable (per RECIST definition in Example 2) and did not experience DLTs or unacceptable toxicity during Cycle 1 may continue to receive additional 8-week treatment cycles with AU-007 plus avelumab until discontinuation criteria as defined in Example 2 are met. Tumor assessments occur on Study Day 56 of each cycle. Response is based on RECIST v1.1 as described in Example 2.

An additional dose or doses of 135,000 IU/kg aldesleukin was administered at Investigator and Sponsor discretion based on objective tumor findings and pharmacodynamic data in patients who are tolerating treatment and are clinically stable as follows:
1. Aldesleukin administered with each cycle (Q8W, Day 1 of cycle) until objective tumor shrinkage is observed on radiologic imaging or physical exam;
2. Aldesleukin administered with objective signs of worsening tumor growth kinetics: e.g., previously shrinking tumors becoming stable, or with new tumor growth.
   Preliminary Results of AU-007 in Combination with Single-Dose Aldesleukin and Avelumab

### Cohort 1 (RP2D-1): 4 Patients Enrolled

1 patient did not complete DLT period and was replaced. No DLT were detected at the observed period.
1 patient discontinued at the end of Cycle 2 with 14% increase in target lesions.
1 patient continues on study in Cycle 4. The patient had a 27% decrease in target lesion at the end of Cycle 1, 45% decrease in target lesion at the end of Cycle 2, and a 36% decrease in target lesion at the end of Cycle 3. A new brain lesion was noted at the end of Cycle 2, which was treated with radiotherapy.
1 patient discontinued at Cycle 2 Day 1 with a 5% increase in target lesion.
   Cohort 2 (RP2D) of Safety Run-In Cohorts: 3 Patients Enrolled. These Patients Were not Treated in Cohort 1 (RP2D-1) Before this Treatment.
1 patient discontinued at end of Cycle 1 with a 46% increase in target lesions
1 patient discontinued at end of Cycle 1 with an 18% increase in target lesions.
1 patient is ongoing in Cycle 2, with a 10% decrease in target lesion at the end of Cycle 1; new brain mets noted.

### Cohort Expansion: 3 Patients Enrolled to Date

1 patient is ongoing in Cycle 2, with a 9% decrease in target lesions at the end of Cycle 1
2 patients are ongoing in Cycle 1 with end of Cycle tumor assessments ˜09/10 September

Cohorts 1 and 2 were the safety run-in cohorts. Patients in any cohort, including safety run-in and expansion, can receive IL-2 boost. No patients enrolled in Part 3 of this study have received an aldeleukin booster to date.

### Example 6

AU-007 in Combination with Single-Dose Aldesleukin and Optional Aldesleukin Boost Administration (Phase 2B)

### Treatment Methods

Patients with unresectable locally advanced or metastatic malignant neoplasms including clear cell renal cell carcinoma (ccRCC), cutaneous melanoma that has progressed during or following treatment with either PD-1 or PD-L1, or unresectable locally advanced or metastatic PD-L1-positive non-small cell lung cancer (NSCLC) were enrolled in Arm 2B of the Cohort Expansion phase of the study.

Unresectable locally advanced tumors cannot be resected and have spread locally (in contrast to metastatic tumors that spread from the original location to another part of the body).

Inclusion criteria for ccRCC patients included patient's cancer progressing during or following at least 2 approved therapeutic regimens (for example but not limited to small molecule inhibitors, anti-PD-1 or anti-PD-L1 therapy).

Inclusion criteria for cutaneous melanoma patients:
Patients with BRAF mutations must either be ineligible for or have refused a BRAF+MEK inhibitor.
Must have objective progression after receiving at least two cycles of prior doublet therapy (anti-PD-1/anti-CTLA-4 or anti-PD-1/anti-LAG-3).
“Objective progression” is considered measurable/quantifiable, i.e., Response Evaluation Criteria in Solid Tumors (RECIST v1.1 definition of progression—see definition below).
Progressive Disease is defined as having at least a 20% increase in the sum of diameters of target lesions, taking as reference the smallest sum on study (this includes the baseline sum if that is the smallest on study). Progression is a >/=20% increase in the sum of the tumors compared to the smallest sum from any prior study scan. In addition to the relative increase of 20%, the sum must also demonstrate an absolute increase of at least 5 mm. The appearance of one or more new lesions is also considered progression.
   Radiographic progression ≥4 weeks prior to the first dose of study drug to rule out late response to most recent therapy. The requirement for documented radiologic progression may be waived after review by Medical Monitor (e.g., in the case of progression beyond 12 weeks after starting a doublet). This study's baseline scan can serve to document confirmed progression to most recent therapy. The most recent therapy is the last therapy the patient received before starting this study.
   Lactate dehydrogenase (LDH)≤2.5×upper limit of normal.

Inclusion criteria for NSCLC patients:
Patients with unresectable locally advanced or metastatic, PD-L1-positive (tumor proportion score [TPS]≥1%), non-small cell lung cancer (NSCLC) not harboring an activating EGFR mutation or ALK rearrangement and has progressed during or following treatment with an anti-PDx (either PD-1 or PD-L1) with or without platinum-based chemotherapy are eligible for enrollment in Part 3 of the study.

Patients received the recommended Phase 2 dose (RP2D) of 9.0 mg/kg AU-007 (administered IV Q2W) plus a single loading dose of 135,000 IU/kg aldesleukin (administered SC) as described in FIG. 2. Patients who remained clinically stable (per RECIST definition as previously elaborated) and do not experience DLTs or unacceptable toxicity during Cycle 1 continue to receive additional 8-week treatment cycles with AU-007 until discontinuation criteria are met. Tumor assessments were conducted on Study Day 56 of each cycle. Response was based on RECIST v1.1 as previously defined.

An additional dose or doses of 135,000 IU/kg aldesleukin were administered at Investigator and Sponsor discretion based on objective tumor findings and pharmacodynamic data in patients who were tolerating treatment and were clinically stable as follows:
1. Aldesleukin administered with each cycle (Q8W, Day 1 of cycle) until objective tumor shrinkage is observed on radiologic imaging or physical exam;
2. Aldesleukin administered with objective signs of worsening tumor growth kinetics: e.g., previously shrinking tumors becoming stable, or with new tumor growth.

Patients who are tolerating treatment and are clinically stable can receive an additional dose of IL-2 (Aldesleukin) on Day 1 of each cycle until objective tumor shrinkage is observed on radiologic imaging or physical exam. The investigator can also administer an additional dose of aldesleukin with objective signs of worsening tumor growth kinetics: e.g., previously shrinking tumors becoming stable, or with new tumor growth.

### Results

### Patients Receiving the RP2D Dose Plus an Additional Dose or Doses of 135,000 IU/Kg Aldesleukin (‘Boost IL-2 Dosing’)

### Patient AU08-0066

Patient AU08-0066 received an IL-2 boost of 135,000 IU/kg on Cycle 4 Day 1 (C4D1;

24 weeks since treatment initiation).

Patient AU08-0066 is a 56-year-old female with metastatic clear cell renal cell carcinoma, diagnosed 11 Apr. 2019 upon right radical nephrectomy, who enrolled in Arm 2B Expansion Cohort (9.0 mg/kg AU-007+135,000 IU/kg aldesleukin loading dose). Prior systemic therapies were adjuvant sunitinib May-November 2019 with progressive disease and 48 cycles of nivolumab 10 Dec. 2019-20 Nov. 2023 with progressive disease.

At screening for study CP-AU-007-01, three baseline target lesions (lung right lower lobe, and two bone lesions) were identified totaling 76 mm, and non-target lesions included axilla, mediastinal, and abdomen lymph nodes and a pancreas mass.

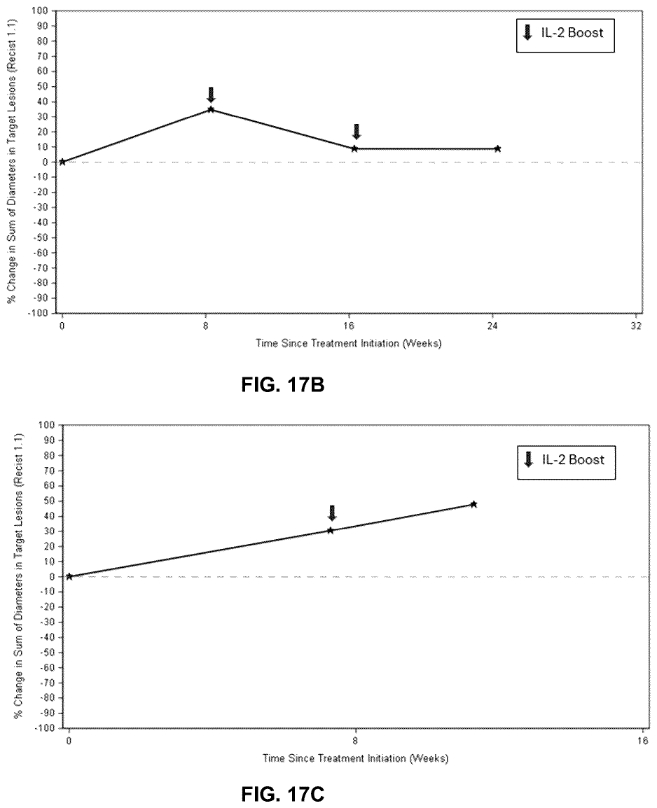

Cycle 1 Day 1 of AU-007+aldesleukin was administered 5 Mar. 2024 (FIG. 17A, Time 0). The patient had stable disease per RECIST at the end of Cycle 1 and Cycle 2 with a 16% decrease in target lesion volume (Table 9). At the end of Cycle 3, the target lesion volume had decreased 17% from baseline (considered stable) and the patient received an additional dose of 135,000 IU/kg aldesleukin on 20 Aug. 2024 (Cycle 4 Day 1; C4D1) to improve upon the response. Target lesions remained stable through Cycle 4 and further decreased by 21% at the end of Cycle 5 (26 Nov. 2024) and Cycle 6 (21 Jan. 2025); decreased by 20% at the end of Cycle 7 (25 Mar. 2025), 22% at the end of Cycle 8 (20 May 2025) (Table 9), and 24% at the end of Cycle 9 (15 Jul. 2025; data not presented in the FIG.). The patient continues on study with stable disease in Cycle 10.

TABLE 9
Target Tumor Volume (mm) at Baseline and End
of Each 8-Week Cycle (% change from baseline)

| | | End of | End of | End of | End of | End of | End of | End of | End of |
|---|---|---|---|---|---|---|---|---|---|
| | | Cycle | Cycle | Cycle | Cycle | Cycle | Cycle | Cycle | Cycle |
| | Baseline* | 1 | 2 | 3** | 4 | 5 | 6 | 7 | 8 |
| Target | 76 | 64 | 64 | 63 | 63 | 60 | 60 | 61 | 59 |
| tumor | | | | | | | | | |
| volume | | | | | | | | | |
| (mm) | | | | | | | | | |
| % | | (−16) | (−16) | (−17) | (−17) | (−21) | (−21) | (−20) | (−22) |
| change | | | | | | | | | |
| from | | | | | | | | | |
| baseline | | | | | | | | | |

*1^{st} AU-007 + loading dose of aldesleukin administered
**Aldesleukin booster

### Patient US01-0098

Patient US01-0098 received IL-2 boosts of 135,000 IU/kg on Cycle 2 Day 1 (C2D1; 8 weeks since treatment initiation) and Cycle 3 Day 1 (C3D1; 16 weeks since treatment initiation).

Patient US01-0098 was a 46-year-old female with metastatic melanoma diagnosed 9 Sep. 2008 who enrolled in Arm 2B Expansion Cohort (9.0 mg/kg AU-007+135,000 IU/kg aldesleukin loading dose). Prior systemic therapy was 4 cycles of ipilimumab/nivolumab 6 Jun. 2024-21 Oct. 2024 with progressive or recurring disease.

At screening for study CP-AU-007-01, the patient had one target lesion (right inguinal lymph node) measuring 23 mm, and non-target lesions of the lung and left para-aortic, left common iliac, and left inguinal lymph nodes. Cycle 1 Day 1 of AU-007+ aldesleukin was administered 25 Nov. 2024. The target tumor progressed (35% increase) at the end of Cycle 1 and was stable compared to baseline (9% increase) at the end of Cycles 2 and 3 (Table 10). The patient continued to Cycle 4, receiving the final AU-007 dose on Cycle 4 Day 29, after which she discontinued treatment and was lost to follow-up for further evaluation. (FIG. 17B)

TABLE 10
[Target Tumor Volume (mm) at Baseline and End of Each 8-Week
Cycle (% change from baseline).

| | | End of Cycle | End of Cycle | End of Cycle |
|---|---|---|---|---|
| | Baseline* | 1 ** | 2 ** | 3 |
| Target tumor | 23 | 31 | 25 | 25 |
| volume (mm) | | | | |
| % change from | | (+35) | (+9) | (+9) |
| baseline | | | | |

*1st AU-007 + loading dose of aldesleukin administered
** Aldesleukin booster administered

TABLE 11
[]Data on Additional Patients that Received an Aldesleukin Booster.

| | | | | | Aldesleuk |
|---|---|---|---|---|---|
| | | | | | in booster |
| | | AU-007 | IL-2 | Patient | timing |
| | | dose and | dose and | Identifier | (cycle and |
| Arm/Cohort | Tumor | schedule | schedule | Code | day) |
| Arm 1B Cohort 2; | Bladder | 4.5 | 45,000 | US03- | C12D15; |
| The patient started | | mg/kg | IU/kg | 0020 | 135,000 |
| at 4.5 mg/kg AU-007 | | Q2W | | | IU/kg |
| and increased to 9 | | | | | |
| mg/kg on | | | | | |
| Cycle 5 day 1. | | | | | |
| Arm 2C: switched | RCC | 9 | 135,000 | AU03- | C4D29, |
| to Arm 2B C3D1 and | | mg/kg | | 0081 | C5D15 |
| discontinued | | Q2W | Q2W | | |
| Q2W IL-2 | | | | | |
| Arm 2C; switched to | Melanoma | 9 | 135,000 | US03- | C4D1 |
| Arm 2B C3D43 and | | mg/kg | Q2W | 0073 | |
| discontinued Q2W | | Q2W | | | |
| IL-2 | | | | | |

### Patient US03-0020—Additional Data

ctDNA or circulating tumor DNA, is small fragments of DNA shed by tumors into the bloodstream. An increasing level of ctDNA during cancer treatment often predicts disease progression, treatment failure, and poor survival outcomes. Monitoring ctDNA levels can provide valuable information on tumor burden and response to therapy, sometimes detecting progression earlier than traditional methods like imaging.

### Ctdna Measurements:

Cycle 2 Day 1 (20 Jun. 2023): 0.00
Cycle 2 Day 29 (18 Jul. 2023): 0.05
Cycle 6 Day 1 (24 Jan. 2024): 0.08
Cycle 12 Day 1 (7 Jan. 2025): 0.12
Cycle 12 Day 43 (25 Feb. 2025): 0.68
Cycle 16 Day 15 (3 Sep. 2025): “negative” per investigator.

At the end of Cycle 11, there was some concern for a slight increase in tumor burden per PET scan, and the ctDNA level was slightly increased. Boost administered 28 Jan. 2025 (Cycle 12 Day 15). The patient remains in stable disease, currently in Cycle 16.

Target Tumor Volumes (mm) at Baseline and End of Each 8-Week Cycle (% change from baseline) for patient US03-0020 are presented below in Table 12. This patient had non-target disease only, meaning not measurable per RECIST so the lesions are only noted and tracked as “present” or “absent.” Patient US03-0020 received loading dose of 45K IU/kg; boost was 135K IU/kg.

The patient started at 4.5 mg/kg AU-007 and increased to 9 mg/kg on Cycle 5 day 1.

TABLE 12
Target Tumor Volumes (mm) at Baseline and End of Each 8-Week
Cycle (% change from baseline)

| | | | Left Iliac |
|---|---|---|---|
| Non-target | | Urinary | Lymph |
| Lesions | | Bladder | Nodes |
| Baseline | 21 Apr 2023 | Present | Present |
| Cycle 1 Day 56 | 19 Jun 2023 | Present | Present |
| Cycle 2 Day 56 | 14 Aug 2023 | Present | Present |
| Cycle 3 Day 56 | 09 Oct 2023 | Present | Present |
| Cycle 4 Day 56 | 04 Dec 2023 | Present | Present |
| Cycle 5 Day 56 | 29 Jan 2024 | Present | Present |
| Cycle 6 Day 56 | 25 Mar 2024 | Present | Present |
| Cycle 7 Day 56 | 20 May 2024 | Present | Absent |
| Cycle 8 Day 56 | 19 Jul 2024 | Present | Absent |
| Cycle 9 Day 56 | 13 Sep 2024 | Present | Absent |
| Cycle 10 Day 56 | 08 Nov 2024 | Present | Absent |
| Cycle 11 Day 56 | 03 Jan 2025 | Present | Absent |
| Cycle 12 Day 56 | 07 Mar 2025 | Present | Absent |
| Cycle 13 Day 56 | 05 May 2025 | Present | Absent |
| Cycle 14 Day 56 | 30 Jun 2025 | Present | Absent |
| Cycle 15 Day 56 | 26 Aug 2025 | Data not | Data not |
| | | entered | entered |

### Patient US03-0073—Additional Data

Patient US03-0073 switched to Arm 2B and discontinued Q2W IL-2 on Cycle 3 Day 43 (9 Oct. 2024), received an IL-2 boost on Cycle 4 Day 1 (23 Oct. 2024), and showed an increase in target tumor size and a new tumor (progressive disease) at Cycle 4 Day 56 (17 Dec. 2024). The patient was discontinued with progressive disease at the end of Cycle 4.

Target Tumor Volumes (mm) at Baseline and End of Each 8-Week Cycle (% change from baseline) for patient US03-0073 are presented below in Table 13.

TABLE 13
Target Tumor Volumes (mm) at Baseline and End of Each 8-Week
Cycle (% change from baseline)

| | | Total Target | Non-target |
|---|---|---|---|
| | | Lesion Size (mm) | lesions (1) |
| Baseline | 23 Apr 2024 | 39 | Present |
| Cycle 1 Day 56 | 27 Jun 2024 | 31 | Present |
| Cycle 2 Day 56 | 26 Aug 2024 | 30 | Present |
| Cycle 3 Day 56 | 22 Oct 2024 | 34 | Present |
| Cycle 4 Day 56 | 17 Dec 2024 | 38 (presence of | Present |
| | | new lesion | |
| | | therefore | |
| | | progressive | |
| | | disease | |

### Patient AU03-0081—Additional Data

Patient AU03-0081 switched to Arm 2B and discontinued Q2W IL-2 on Cycle 3 Day 1 (21 Oct. 2024). The patient received IL-2 boosts on Cycle 4 Day 29 (13 Jan. 2025) and

Cycle 5 Day 15 (26 Feb. 2025) with initial increasing tumor size, then showed a decrease in target tumor size on the Cycle 7 Day 56 scan (25 Jul. 2025); therefore, the boost administration may be considered beneficial.

Target Tumor Volumes (mm) at Baseline and End of Each 8-Week Cycle (% change from baseline) for patient AU03-0081 are presented below in Table 14.

TABLE 14
Target Tumor Volumes (mm) at Baseline and End of Each 8-Week
Cycle (% change from baseline)

| | | Total Target | |
|---|---|---|---|
| | | Lesion Size | Non-target |
| | | (mm) | lesions (2) |
| Baseline | 03 Jun 2024 | 69 | Present |
| Cycle 1 Day 56 | 20 Aug 2024 | 64 | Present |
| Cycle 2 Day 56 | 16 Oct 2024 | 70 | Present |
| Cycle 3 Day 56 | 12 Dec 2024 | 75 | Present |
| Cycle 4 Day 56 | Not done | — | — |
| Cycle 5 Day 56 | 07 Apr 2025 | 79 | Present |
| Cycle 6 Day 56 | 29 May 2025 | 78 | Present |
| Cycle 7 Day 56 | 25 Jul 2025 | 66 | Present |
| Cycle 8 Day 56 | Pending | | |

The patient remains on treatment in Cycle 8.

### Patient AU03-0084

Patient AU03-0084 received an IL-2 boost of 135,000 IU/kg on Cycle 2 Day 1 (C2D1;

8 weeks since treatment initiation).

Patient AU03-0084 was a 77-year-old female with metastatic melanoma, initially diagnosed with locally resectable disease in 1983 with first evidence of metastasis in December 2021, who enrolled in Arm 2B Expansion Cohort (9.0 mg/kg AU-007+135,000 IU/kg aldesleukin loading dose). Prior systemic therapies were neoadjuvant ipilimumab/nivolumab December 2021-May 2022 with a complete response, and lifirafenib/mirdametinib 2023-2024 with progressive or recurring disease.

At screening for study CP-AU-007-01, three target tumor lesions were identified (right upper breast, abdominal wall, mesenteric lymph node nodule) totaling 46 mm, and non-target lesions of right groin and presacral lymph nodes. Cycle 1 Day 1 of AU-007+aldesleukin was administered 30 Jul. 2024. The patient had progressive disease at the end of Cycle 1, with a 30% increase in target tumors. The patient continued to Cycle 2 and received an aldesleukin boost of 135,000 IU/kg on Cycle 2 Day 1 (C2D1) (FIG. 17C). The patient discontinued with progressive disease on Cycle 2 Day 56.

TABLE 15

| Target Tumor Volume (mm) at Baseline and End of Cycle 1 | | |
|---|---|---|
| and Cycle 2 Day 15 (% change from baseline)*, **. | | |
| Targettumor volume (mm) at baseline, end of Cycle 1, and | | |
| Cycle 2 Day 15 (3% change from baseline) | | |
| 46 | 60 | 68 |
| | (+30) | (+48) |

*1st AU-007 + loading dose of aldesleukin administered; Target tumor volume at baselinewas 46 mm.
**Aldesleukin booster administered C2D1; End of cycle 1; target tumor volume was 60 mm

These optional Aldesleukin (IL-2) Boost Administration (Phase 2B) studies remain ongoing.

### Example 7

AU-007, a Human Monoclonal Antibody (mAb) that Binds to IL-2 and Inhibits CD25 Binding, Plus Low-Dose Aldesleukin: Phase 2 Update on Melanoma and Non-Small Cell Lung Cancer (NSCLC)

Background: AU-007 is a monoclonal antibody (mAb) that binds to the interleukin-2 (IL-2) cytokine at an epitope inhibiting its interaction with CD25 on the IL-2 receptor while allowing engagement with the dimeric receptor complex, CD122/CD132. Thus AU-007 bound IL-2 expands Teff and NK cell populations (dimeric IL-2R) without expanding Tregs (trimeric IL-2R). Avelumab is an anti-PD-L1 mAb containing an active fragment crystallizable Fc region. Avelumab can potentially initiate antibody-dependent cell-mediated cytotoxicity mediated by NK cells, thus bringing innate and adaptive immunity against a tumor.

### Methods

The study consists of Phase (Ph) 1 dose escalation and four ongoing Ph2 expansion cohorts evaluating 8-week cycles of the recommended Ph2 dose (RP2D) of 9 mg/kg AU-007 IV Q2W+ one subcutaneous (SC) 135K IU/kg aldesleukin dose on Day 1. Ph2 Cohorts 1 and 4 evaluate melanoma at RP2D±nivolumab (+nivolumab not reported); Cohorts 2 and 3 evaluate PD-L1-positive (≥1%) NSCLC that has progressed on anti-PD-1/L1±chemotherapy at RP2D, ±avelumab.

### Results

Ninety-eight patients enrolled as of 30 May 2025. AU-007+ aldesleukin was well-tolerated in Ph1 with no dose-limiting toxicity. Twelve melanoma patients enrolled in Ph2 at the RP2D and two melanoma patients received AU-007 and single dose aldesleukin below the RP2D in Ph1. Two melanoma patients refractory to anti-CTLA-4 and PD-1 therapy had tumor reductions of 48% (14 months on treatment) and 100% (continues treatment at 16 months). A melanoma patient refractory to anti-PD-1+relatlimab therapy had a 58% reduction and continues treatment at 13 months.

Early NSCLC activity was seen in a Ph1 patient who failed prior anti-PD-L1 therapy and received AU-007 Q2W monotherapy with a 14% tumor reduction. In Ph2, two NSCLC patients have received the RP2D; one patient is ongoing with stable disease (SD) at five months. Six NSCLC patients received avelumab plus the RP2D-1 (four patients; one with 45% target lesion reduction, two with SD, one pending evaluation) or the RP2D (two patients; one with progressive disease, one pending evaluation).

The most common treatment-related adverse events were Grade ½ fatigue (18%), chills (18%), pyrexia (17%), infusion-related reaction (13%), and nausea (11%).

### CONCLUSIONS

AU-007+ low-dose SC aldesleukin exhibits a manageable toxicity profile with deep tumor reductions and durability in melanoma, and early signs of activity in the more recently opened NSCLC cohorts. Ph2 cohorts evaluating melanoma (+nivolumab) and NSCLC (±avelumab) are ongoing.

What is claimed is:

1. A combination therapy for treating unresectable locally advanced or metastatic non-small cell lung cancer (NSCLC) that is PD-L1 positive wherein treatment consists of 2^{nd} line or 3^{rd} line treatment, the combination therapy comprising an anti-IL-2 antibody or a pharmaceutical composition thereof, a loading dose of IL-2 or a pharmaceutical composition thereof, and avelumab or a pharmaceutical composition thereof,
wherein said IL-2 antibody comprises a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3,
wherein said HCDR1 comprises the amino acid sequence of SEQ ID NO:62, said HCDR2 comprises the amino acid sequence of SEQ ID NO:63, said HCDR3 comprises the amino acid sequence of SEQ ID NO:64, said LCDR1 comprises the amino acid sequence of SEQ ID NO:65, said LCDR2 comprises the amino acid sequence of DAS, and said LCDR3 comprises the amino acid sequence of SEQ ID NO: 67;
wherein said anti-IL-2 antibody is formulated for administration at a dose of 9 mg/kg of a subject's body weight, the loading dose of IL-2 is formulated for subcutaneous administration at a dose of 135,000 IU/kg of a subject's body weight, and said avelumab is formulated for administration at a dose of 800 mg; and
wherein said pharmaceutical composition(s) further comprises a pharmaceutically acceptable carrier.

2. The combination therapy according to claim 1, wherein the amino acid sequence of the VH comprises the amino acid sequence of SEQ ID NO:26 and the amino acid sequence of VL comprises the amino acid sequence of SEQ ID NO:27.

3. The combination therapy according to claim 1, wherein the amino acid sequence of the full length heavy chain is set forth in SEQ ID NO:72 and the amino acid sequence of the full length light chain is set forth in SEQ ID NO:73.

4. The combination therapy according to claim 1, wherein the antibody comprises an IgG, IgA, IgM, IgE, IgD, a Fv, a scFv, a Fab, a F(ab′)₂, a minibody, a diabody, or a triabody.

5. The combination therapy according to claim 1, wherein said antibody comprises a heavy chain comprising a mutation that reduces binding to fragment crystallizable gamma receptors (FcγRs), wherein reduced binding is compared with said antibody lacking the mutation in the heavy chain that affects FcγRs binding.

6. The combination therapy according to claim 5, wherein said mutation comprises L234A, L235A (LALA) mutations.

7. A method of treating an unresectable locally advanced or metastatic non-small cell lung cancer (NSCLC) that is PD-L1 positive in a subject, said method comprising administering to said subject an anti-IL-2 antibody at a dose of 9 mg/kg of said subject's body weight or a pharmaceutical composition thereof, a loading dose of 135,000 IU/kg of said subject's body weight of IL-2 or a pharmaceutical composition thereof, and avelumab at a dose of 800 mg or a pharmaceutical composition thereof, said IL-2 antibody comprising a heavy chain variable region (VH) comprising heavy chain complementarity determining regions (HCDRs) HCDR1, HCDR2 and HCDR3 and a light chain variable region (VL) comprising light chain complementarity determining regions (LCDRs) LCDR1, LCDR2 and LCDR3,
wherein said HCDR1 comprises the amino acid sequence of SEQ ID NO:62, said HCDR2 comprises the amino acid sequence of SEQ ID NO:63, said HCDR3 comprises the amino acid sequence of SEQ ID NO:64, said LCDR1 comprises the amino acid sequence of SEQ ID NO:65, said LCDR2 comprises the amino acid sequence of DAS, and said LCDR3 comprises the amino acid sequence of SEQ ID NO: 67;
wherein said NSCLC comprises unresectable locally advanced or metastatic NSCLC that is PD-L1 positive;
wherein said treating comprises second-line or third-line treatment;
wherein said pharmaceutical composition(s) further comprises a pharmaceutically acceptable carrier,
thereby treating said NSCLC in said subject.

8. The method according to claim 7, wherein the administration of said loading dose of IL-2 is prior to, concurrent with, or following the administration of said anti-IL-2 antibody, said avelumab, or both.

9. The method according to claim 7, further comprising the step of administering one or more additional doses of said anti-IL-2 antibody or a pharmaceutical composition thereof, wherein said pharmaceutical composition further comprises a pharmaceutically acceptable carrier.

10. The method according to claim 9, wherein said additional doses of said anti-IL-2 antibody or a pharmaceutical composition thereof are administered to said subject once every two weeks.

11. The method according to claim 7, further comprising the step of administering one or more additional doses of said avelumab or a pharmaceutical composition thereof, wherein said pharmaceutical composition further comprises a pharmaceutically acceptable carrier.

12. The method according to claim 11, wherein said one or more additional doses of said avelumab or a pharmaceutical composition thereof are administered to said subject once every two weeks.

13. The method according to claim 7, further comprising the step of administering one or more booster doses of IL-2 or a pharmaceutical composition thereof, wherein said pharmaceutical composition further comprises a pharmaceutically acceptable carrier.

14. The method according to claim 13, wherein the administration of said one or more booster doses of IL-2 is prior to, concurrent with, or following the administration of said one or more additional doses of said anti-IL-2 antibody, one or more additional doses of said avelumab, or both.

15. The method according to claim 13, wherein at least one of the one or more IL-2 booster doses is administered at a dose of 135,000 IU/kg of said subject's body weight.

16. The method according to claim 13, wherein said at least one booster dose is administered to said subject if tumor volume is stable, if one or more previously shrinking tumors becomes stable, if there is an increase in one or more tumor markers, if one or more new tumors are detected, if tumor growth is detected in one or more tumors that had previously been stable or had decreased in size, or any combination thereof.

17. The method according to claim 7, wherein said method comprises (i) reducing the size of the tumor, (ii) inhibiting or reducing growth of the tumor, (iii) inhibiting or reducing metastases of said tumor, (iv) inhibiting the production of new lesions, (v) decreasing or shrinking target lesions, (vi) eliminating target lesions, or (vii) any combination thereof.

18. The method according to claim 7, wherein said NSCLC progressed after prior checkpoint inhibitor therapy in said subject.

19. The method according to claim 7 wherein said NSCLC comprises squamous NSCLC.

20. The method according to claim 7, wherein said NSCLC comprises non-squamous NSCLC.


SEQUENCE LISTING (73 sequences)

SEQ ID NO: 1 - DNA, 747 bp, synthetic construct
  misc_feature 1..747: note = scFv clone 17.021
  source 1..747: mol_type = other DNA
gagattcagctgcagcagagcggtccagaactgcgtcgtccaggctcctccgttaaactgagctgcaaagcctctggttataacatcaccgataacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgttctctggattccacctatatttacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactaccagagcccaccaactttcggtgcaggcaccaaactggaactgaaa

SEQ ID NO: 2 - DNA, 747 bp, synthetic construct
  misc_feature 1..747: note = scFv clone 17.022
  source 1..747: mol_type = other DNA
gagattcagctgcagcagagcggtccagaactgcgtcgtccaggctcctccgttaaactgagctgcaaagcctctggttataacatcaccgattacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgttctctggattccacctggatttacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactacattaccccaccaactttcggtgcaggcaccaaactggaactgaaa

SEQ ID NO: 3 - DNA, 747 bp, synthetic construct
  misc_feature 1..747: note = scFv clone 17.023
  source 1..747: mol_type = other DNA
gagattcagctgcagcagagcggtccagaactgcgtcgtccaggctcctccgttaaactgagctgcaaagcctctggttataacatcaccgattacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgtcagctggattccacctatatttacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactacattaccccaccaactttcggtgcaggcaccaaactggaactgaaa

SEQ ID NO: 4 - DNA, 747 bp, synthetic construct
  misc_feature 1..747: note = scFv clone 17.030
  source 1..747: mol_type = other DNA
gagattcagctgcagcagagcggtccagaactgcgtcgtccaggctcctccgttaaactgagctgcaaagcctctggttataacatcaccgattacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgttctctggattccacctatgactacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactacatttcgccaccaactttcggtgcaggcaccaaactggaactgaaa

SEQ ID NO: 5 - DNA, 747 bp, synthetic construct
  misc_feature 1..747: note = scFv clone 17.035
  source 1..747: mol_type = other DNA
gagattcagctgcagcagagcggtccagaactgcgtcgtccaggctcctccgttaaactgagctgcaaagcctctggttataacatcaccgattacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgttctctggattccacctataactacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactggattagcccaccaactttcggtgcaggcaccaaactggaactgaaa

SEQ ID NO: 6 - protein, 121 aa, synthetic construct
  REGION 1..121: note = Heavy chain variable region of JES6.1
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARSLDSTYIYPFAYWGQGTLVTVSS

SEQ ID NO: 7 - protein, 113 aa, synthetic construct
  REGION 1..113: note = Light chain variable region of JES6.1
DIVMTQSPFSLAVSEGEMVTINCKSSQSLLSSGNQKNYLAWYQQKPGQSPKLLIYYASTGQSGVPDRFIGSGSGTDFTLTISDVQAEDLADYYCLQHYISPPTFGAGTKLELK

SEQ ID NO: 8 - protein, 121 aa, synthetic construct; identical to SEQ ID NO: 6
  REGION 1..121: note = Heavy chain variable region of JES6.1RMC

SEQ ID NO: 9 - protein, 113 aa, synthetic construct; identical to SEQ ID NO: 7
  REGION 1..113: note = Light chain variable region of JES6.1RMC

SEQ ID NO: 10 - protein, 121 aa, synthetic construct
  REGION 1..121: note = Heavy chain variable region of clone 17.021
EIQLQQSGPELRRPGSSVKLSCKASGYNITDNLIHWVRHRPEHGLEWIGWIDPEDGETRYAQKFQSKATLTADTSSNAAYMQLSSLTPEDTATYFCARSLDSTYIYPFAYWGQGTLVTVSS

SEQ ID NO: 11 - protein, 113 aa, synthetic construct
  REGION 1..113: note = Light chain variable region of clone 17.021
DIVMTQSPFSLAVSEGEMVTINCKSSQSLLRSGNQKNYLAWYQQKPGQSPKLLIYYASTGQSGVPDRFIGSGSGTDFTLTISDVQAEDLADYYCLQHYQSPPTFGAGTKLELK

SEQ ID NO: 12 - protein, 121 aa, synthetic construct
  REGION 1..121: note = Heavy chain variable region of clone 17.022
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARSLDSTWIYPFAYWGQGTLVTVSS

SEQ ID NO: 13 - protein, 113 aa, synthetic construct
  REGION 1..113: note = Light chain variable region of clone 17.022
DIVMTQSPFSLAVSEGEMVTINCKSSQSLLRSGNQKNYLAWYQQKPGQSPKLLIYYASTGQSGVPDRFIGSGSGTDFTLTISDVQAEDLADYYCLQHYITPPTFGAGTKLELK

SEQ ID NO: 14 - protein, 121 aa, synthetic construct
  REGION 1..121: note = Heavy chain variable region of clone 17.023
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARQLDSTYIYPFAYWGQGTLVTVSS

SEQ ID NO: 15 - protein, 113 aa, synthetic construct; identical to SEQ ID NO: 13
  REGION 1..113: note = Light chain variable region of clone 17.023

SEQ ID NO: 16 - protein, 121 aa, synthetic construct
  REGION 1..121: note = Heavy chain variable region of clone 17.030
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARSLDSTYDYPFAYWGQGTLVTVSS

SEQ ID NO: 17 - protein, 113 aa, synthetic construct
  REGION 1..113: note = Light chain variable region of clone 17.030
DIVMTQSPFSLAVSEGEMVTINCKSSQSLLRSGNQKNYLAWYQQKPGQSPKLLIYYASTGQSGVPDRFIGSGSGTDFTLTISDVQAEDLADYYCLQHYISPPTFGAGTKLELK

SEQ ID NO: 18 - protein, 121 aa, synthetic construct
  REGION 1..121: note = Heavy chain variable region of clone 17.035
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARSLDSTYNYPFAYWGQGTLVTVSS

SEQ ID NO: 19 - protein, 113 aa, synthetic construct
  REGION 1..113: note = Light chain variable region of clone 17.035
DIVMTQSPFSLAVSEGEMVTINCKSSQSLLRSGNQKNYLAWYQQKPGQSPKLLIYYASTGQSGVPDRFIGSGSGTDFTLTISDVQAEDLADYYCLQHWISPPTFGAGTKLELK

SEQ ID NO: 20 - protein, 121 aa, synthetic construct
  REGION 1..121: note = Heavy chain variable region of clone 17.038
QVQLVQSGAEVKKPGSSVKVSCKASGYSITDSLIHWVRQAPGQGLEWMGWIDPEDGEINYAQKFQGRVTLTADTSTSTAYMELSSLRSEDTAVYYCARSLDSYPIDPFAYWGQGTLVTVSS

SEQ ID NO: 21 - protein, 113 aa, synthetic construct
  REGION 1..113: note = Light chain variable region of clone 17.038
DIVMTQSPDSLAVSLGERATINCKSSQSLLRNGNQQNYLAWYQQKPGQPPKLLIYYASTGQSGVPDRFSGSGSGTDFTLTISSLQAEDVAVYYCQQHYVTPPTFGAGTKVEIK

SEQ ID NO: 22 - protein, 121 aa, synthetic construct
  REGION 1..121: note = Heavy chain variable region of clone 17.043
QVQLVQSGAEVKKPGSSVKVSCKASGYSITDYLIHWVRQAPGQGLEWMGWIDPEDGVTAYAQKFQGRVTLTADTSTSTAYMELSSLRSEDTAVYYCARSLDYDPIYPFAYWGQGTLVTVSS

SEQ ID NO: 23 - protein, 113 aa, synthetic construct
  REGION 1..113: note = Light chain variable region of clone 17.043
DIVMTQSPDSLAVSLGERATINCKSSQSLLRNGNQKNYLAWYQQKPGQPPKLLIYYASTGQSGVPDRFSGSGSGTDFTLTISSLQAEDVAVYYCQQHYVTPPTFGAGTKVEIK

SEQ ID NO: 24 - protein, 121 aa, synthetic construct
  REGION 1..121: note = Heavy chain variable region of clone 17.053
QVQLVQSGAEVKKPGSSVKVSCKASGYSITDYLIHWVRQAPGQGLEWMGWIDPEDGETNYAQKFQGRVTLTADTSTSTAYMELSSLRSEDTAVYYCARSLDYDPIYPFAYWGQGTLVTVSS

SEQ ID NO: 25 - protein, 113 aa, synthetic construct
  REGION 1..113: note = Light chain variable region of clone 17.053
DIVMTQSPDSLAVSLGERATINCKSSQSLLRNGNQKNYLAWYQQKPGQPPKLLIYYASTGQSGVPDRFSGSGSGTDFTLTISSLQAEDVAVYYCQQHYITPPTFGAGTKVEIK

SEQ ID NO: 26 - protein, 121 aa, synthetic construct
  REGION 1..121: note = Heavy chain variable region of clone 17.054
QVQLVQSGAEVKKPGSSVKVSCKASGYSITDDLIHWVRQAPGQGLEWMGWIDPEDGETNYAQKFQGRVTLTADTSTSTAYMELSSLRSEDTAVYYCARSLDSTWIYPFAYWGQGTLVTVSS

SEQ ID NO: 27 - protein, 113 aa, synthetic construct
  REGION 1..113: note = Light chain variable region of clone 17.054
DIVMTQSPDSLAVSLGERATINCKSSQSLLRRGNQKNHLAWYQQKPGQPPKLLIYDASTGQSGVPDRFSGSGSGTDFTLTISSLQAEDVAVYYCLQSYITPPTFGAGTKVEIK

SEQ ID NO: 28 - DNA, 747 bp, synthetic construct
  misc_feature 1..747: note = scFv clone 17.002
  source 1..747: mol_type = other DNA
gagattcagctgcagcagagcggtccagaactgcgtcgtccaggctcctccgttaaactgagctgcaaagcctctggttataacatcaccgattacctgattcattgggttcgtcaccgtccagaacacggtctggaatggatcggctggattgatccagaggacggtgaaacccgttacgcccaaaaattccagtctaaagcaaccctgactgccgacactagctccaacgccgcttacatgcagctgagcagcctgacccctgaggataccgccacctatttctgcgcacgttctctggattccaccccgatttacccattcgcatactggggccaaggcacccttgtcaccgtttcctccggcggtggtggtagcggaggcggaggatcaggtggaggcggcagtgacattgttatgactcagtccccattctccctggccgtttctgagggtgaaatggtaaccattaactgcaaatcatcccagtccctgctgcggtctggcaaccagaaaaactatctggcctggtaccagcagaaaccaggtcagtccccaaaactgctgatctactacgcatctactggccagtctggtgtcccagaccgtttcattggctctggctctggtaccgatttcaccctgaccatcagcgatgttcaagctgaagacctggccgattactattgtctgcagcactacattacgccaccaactttcggtgcaggcaccaaactggaactgaaa

SEQ ID NO: 29 - protein, 121 aa, synthetic construct
  REGION 1..121: note = Heavy chain variable region of clone 17.002
EIQLQQSGPELRRPGSSVKLSCKASGYNITDYLIHWVRHRPEHGLEWIGWIDPEDGETRYAQKFQSKATLTADTSSNAAYMQLSSLTPEDTATYFCARSLDSTPIYPFAYWGQGTLVTVSS

SEQ ID NO: 30 - protein, 113 aa, synthetic construct; identical to SEQ ID NO: 13
  REGION 1..113: note = Light chain variable region of clone 17.002

SEQ ID NO: 31 - DNA, 747 bp, synthetic construct
  misc_feature 1..747: note = scFv clone 17.014
  source 1..747: mol_type = other DNA
caggtccaactggtgcagtccggtgccgaagttaaaaaacctgggtcttccgttaaagtttcttgcaaagcctctggctacagcatcaccgattatctgattcactgggtccgtcaggctccaggtcaaggtctggaatggatgggttggatcgatccagaagacggtgaaaccaactatgcccagaaattccagggtcgtgtaaccctgaccgccgacacctccacctctaccgcctacatggagttaagtagcctgcgttcagaggataccgcagtgtactactgcgctcgttcactggactccaccccaatctacccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgttctggtaatcagaaaaactaccttgcatggtatcagcagaaaccaggtcagccaccaaaattactgatctattatgcatctaccggtcaaagcggtgtcccagatcgtttcagcggttccggctccggtactgacttcaccctgaccatctcttcccttcaggccgaagatgtggccgtgtattactgcctgcagcactacatcaccccacctactttcggtgctggtactaaagttgaaatcaaa

SEQ ID NO: 32 - DNA, 747 bp, synthetic construct
  misc_feature 1..747: note = scFv clone 17.038
  source 1..747: mol_type = other DNA
caggtccaactggtgcagtccggtgccgaagttaaaaaacctgggtcttccgttaaagtttcttgcaaagcctctggctacagcatcaccgattctctgattcactgggtccgtcaggctccaggtcaaggtctggaatggatgggttggatcgatccagaagacggtgaaattaactatgcccagaaattccagggtcgtgtaaccctgaccgccgacacctccacctctaccgcctacatggagttaagtagcctgcgttcagaggataccgcagtgtactactgcgctcgttcactggactcctacccaatcgatccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgtaatggtaatcagcaaaactaccttgcatggtatcagcagaaaccaggtcagccaccaaaattactgatctattatgcatctaccggtcaaagcggtgtcccagatcgtttcagcggttccggctccggtactgacttcaccctgaccatctcttcccttcaggccgaagatgtggccgtgtattactgccagcagcactacgtcaccccacctactttcggtgctggtactaaagttgaaatcaaa

SEQ ID NO: 33 - DNA, 747 bp, synthetic construct
  misc_feature 1..747: note = scFv clone 17.043
  source 1..747: mol_type = other DNA
caggtccaactggtgcagtccggtgccgaagttaaaaaacctgggtcttccgttaaagtttcttgcaaagcctctggctacagcatcaccgattatctgattcactgggtccgtcaggctccaggtcaaggtctggaatggatgggttggatcgatccagaagacggtgtgaccgcctatgcccagaaattccagggtcgtgtaaccctgaccgccgacacctccacctctaccgcctacatggagttaagtagcctgcgttcagaggataccgcagtgtactactgcgctcgttcactggactacgacccaatctacccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgtaacggtaatcagaaaaactatcttgcatggtatcagcagaaaccaggtcagccaccaaaattactgatctattatgcatctaccggtcaaagcggtgtcccagatcgtttcagcggttccggctccggtactgacttcaccctgaccatctcttcccttcaggccgaagatgtggccgtgtattactgccagcagcactacgtcaccccacctactttcggtgctggtactaaagttgaaatcaaa

SEQ ID NO: 34 - DNA, 747 bp, synthetic construct
  misc_feature 1..747: note = scFv clone 17.053
  misc_difference 726: note = n is a, c, g, or t
  source 1..747: mol_type = other DNA
caggtccaactggtgcagtccggtgccgaagttaaaaaacctgggtcttccgttaaagtttcttgcaaagcctctggctacagcatcaccgattatctgattcactgggtccgtcaggctccaggtcaaggtctggaatggatgggttggatcgatccagaagacggtgaaactaactatgcccagaaattccagggtcgtgtaaccctgaccgccgacacctccacctctaccgcctacatggagttaagtagcctgcgttcagaggataccgcagtgtactactgcgctcgttcactggactacgacccaatctacccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgtaacggtaatcagaaaaactaccttgcatggtatcagcagaaaccaggtcagccaccaaaattactgatctattatgcatctaccggtcaaagcggtgtcccagatcgtttcagcggttccggctccggtactgacttcaccctgaccatctcttcccttcaggccgaagatgtggccgtgtattactgccagcagcactacattaccccacctactttcggtgcnggtactaaagttgaaatcaaa

SEQ ID NO: 35 - DNA, 747 bp, synthetic construct
  misc_feature 1..747: note = scFv clone 17.054
  source 1..747: mol_type = other DNA
caggtccaactggtgcagtccggtgccgaagttaaaaaacctgggtcttccgttaaagtttcttgcaaagcctctggctacagcatcaccgatgacctgattcactgggtccgtcaggctccaggtcaaggtctggaatggatgggttggatcgatccagaagacggtgaaaccaactatgcccagaaattccagggtcgtgtaaccctgaccgccgacacctccacctctaccgcctacatggagttaagtagcctgcgttcagaggataccgcagtgtactactgcgctcgttcactggactccacctggatctacccattcgcatactggggtcagggcaccctggtaaccgttagtagcggcggtggtggtagcggaggcggaggatcaggtggaggcggcagtgacatcgtgatgacccagtctcctgactccttggccgtctctctgggcgaacgtgcaactatcaactgcaaatccagccagagcttactgcgtcgcggtaatcagaaaaaccaccttgcatggtatcagcagaaaccaggtcagccaccaaaattactgatctatgacgcatctaccggtcaaagcggtgtcccagatcgtttcagcggttccggctccggtactgacttcaccctgaccatctcttcccttcaggccgaagatgtggccgtgtattactgcctgcagagctacatcaccccacctactttcggtgctggtactaaagttgaaatcaaa

SEQ ID NO: 36 - protein, 121 aa, synthetic construct
  REGION 1..121: note = Heavy chain variable region of clone 17.014
QVQLVQSGAEVKKPGSSVKVSCKASGYSITDYLIHWVRQAPGQGLEWMGWIDPEDGETNYAQKFQGRVTLTADTSTSTAYMELSSLRSEDTAVYYCARSLDSTPIYPFAYWGQGTLVTVSS

SEQ ID NO: 37 - protein, 113 aa, synthetic construct
  REGION 1..113: note = Light chain variable region of clone 17.014
DIVMTQSPDSLAVSLGERATINCKSSQSLLRSGNQKNYLAWYQQKPGQPPKLLIYYASTGQSGVPDRFSGSGSGTDFTLTISSLQAEDVAVYYCLQHYITPPTFGAGTKVEIK

SEQ ID NO: 38 - protein, 8 aa, synthetic construct
  REGION 1..8: note = 17.014 H- CDR1
GYSITDYL

SEQ ID NO: 39 - protein, 8 aa, synthetic construct
  REGION 1..8: note = 17.014 H- CDR2
IDPEDGET

SEQ ID NO: 40 - protein, 14 aa, synthetic construct
  REGION 1..14: note = 17.014 H- CDR3
ARSLDSTPIYPFAY

SEQ ID NO: 41 - protein, 12 aa, synthetic construct
  REGION 1..12: note = 17.014 L- CDR1
QSLLRSGNQKNY

SEQ ID NO: 42 - skipped

SEQ ID NO: 43 - protein, 9 aa, synthetic construct
  REGION 1..9: note = 17.014 L- CDR3
LQHYITPPT

SEQ ID NO: 44 - protein, 8 aa, synthetic construct
  REGION 1..8: note = 17.038 H- CDR1
GYSITDSL

SEQ ID NO: 45 - protein, 8 aa, synthetic construct
  REGION 1..8: note = 17.038 H- CDR2
IDPEDGEI

SEQ ID NO: 46 - protein, 14 aa, synthetic construct
  REGION 1..14: note = 17.038 H- CDR3
ARSLDSYPIDPFAY

SEQ ID NO: 47 - protein, 12 aa, synthetic construct
  REGION 1..12: note = 17.038 L- CDR1
QSLLRNGNQQNY

SEQ ID NO: 48 - skipped

SEQ ID NO: 49 - protein, 9 aa, synthetic construct
  REGION 1..9: note = 17.038 L- CDR3
QQHYVTPPT

SEQ ID NO: 50 - protein, 8 aa, synthetic construct
  REGION 1..8: note = 17.043 H- CDR1
GYSITDYL

SEQ ID NO: 51 - protein, 8 aa, synthetic construct
  REGION 1..8: note = 17.043 H- CDR2
IDPEDGVT

SEQ ID NO: 52 - protein, 14 aa, synthetic construct
  REGION 1..14: note = 17.043 H- CDR3
ARSLDYDPIYPFAY

SEQ ID NO: 53 - protein, 12 aa, synthetic construct
  REGION 1..12: note = 17.043 L- CDR1
QSLLRNGNQKNY

SEQ ID NO: 54 - skipped

SEQ ID NO: 55 - protein, 9 aa, synthetic construct
  REGION 1..9: note = 17.043 L- CDR3
QQHYVTPPT

SEQ ID NO: 56 - protein, 8 aa, synthetic construct
  REGION 1..8: note = 17.053 H- CDR1
GYSITDYL

SEQ ID NO: 57 - protein, 8 aa, synthetic construct
  REGION 1..8: note = 17.053 H- CDR2
IDPEDGET

SEQ ID NO: 58 - protein, 14 aa, synthetic construct
  REGION 1..14: note = 17.053 H- CDR3
ARSLDYDPIYPFAY

SEQ ID NO: 59 - protein, 12 aa, synthetic construct
  REGION 1..12: note = 17.053 L- CDR1
QSLLRNGNQKNY

SEQ ID NO: 60 - skipped

SEQ ID NO: 61 - protein, 9 aa, synthetic construct
  REGION 1..9: note = 17.053 L- CDR3
QQHYITPPT

SEQ ID NO: 62 - protein, 8 aa, synthetic construct
  REGION 1..8: note = 17.054 H- CDR1
GYSITDDL

SEQ ID NO: 63 - protein, 8 aa, synthetic construct
  REGION 1..8: note = 17.054 H- CDR2
IDPEDGET

SEQ ID NO: 64 - protein, 14 aa, synthetic construct
  REGION 1..14: note = 17.054 H- CDR3
ARSLDSTWIYPFAY

SEQ ID NO: 65 - protein, 12 aa, synthetic construct
  REGION 1..12: note = 17.054 L- CDR1
QSLLRRGNQKNH

SEQ ID NO: 66 - skipped

SEQ ID NO: 67 - protein, 9 aa, synthetic construct
  REGION 1..9: note = 17.054 L- CDR3
LQSYITPPT

SEQ ID NO: 68 - protein, 451 aa, synthetic construct
  REGION 1..451: note = BDG17.066 heavy Chain LALA
QVQLVQSGAEVKKPGSSVKVSCKASGYSITDSLIHWVRQAPGQGLEWMGWIDPEDGEINYAQKFQGRVTLTADTSTSTAYMELSSLRSEDTAVYYCARSLDSYPIDPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 220 aa, synthetic construct
  REGION 1..220: note = BDG17.066 Light Chain
DIVMTQSPDSLAVSLGERATINCKSSQSLLRNGNQQNYLAWYQQKPGQPPKLLIYYASTGQSGVPDRFSGSGSGTDFTLTISSLQAEDVAVYYCQQHYVTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 451 aa, synthetic construct
  REGION 1..451: note = BDG17.067 heavy Chain LALA
QVQLVQSGAEVKKPGSSVKVSCKASGYSITDYLIHWVRQAPGQGLEWMGWIDPEDGVTAYAQKFQGRVTLTADTSTSTAYMELSSLRSEDTAVYYCARSLDYDPIYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 220 aa, synthetic construct
  REGION 1..220: note = BDG17.067 Light Chain
DIVMTQSPDSLAVSLGERATINCKSSQSLLRNGNQKNYLAWYQQKPGQPPKLLIYYASTGQSGVPDRFSGSGSGTDFTLTISSLQAEDVAVYYCQQHYVTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 451 aa, synthetic construct
  REGION 1..451: note = BDG17.069 heavy Chain LALA
QVQLVQSGAEVKKPGSSVKVSCKASGYSITDDLIHWVRQAPGQGLEWMGWIDPEDGETNYAQKFQGRVTLTADTSTSTAYMELSSLRSEDTAVYYCARSLDSTWIYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 220 aa, synthetic construct
  REGION 1..220: note = BDG17.069 Light Chain
DIVMTQSPDSLAVSLGERATINCKSSQSLLRRGNQKNHLAWYQQKPGQPPKLLIYDASTGQSGVPDRFSGSGSGTDFTLTISSLQAEDVAVYYCLQSYITPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC